(19) United States
(12) Patent Application Publication
(10) Pub. No.: US 2019/0248885 A1
(43) Pub. Date: Aug. 15, 2019

(54) Neuronal and Oligodendrocyte Survival Modulation

(71) Applicant: The Board of Trustees of the Leland Stanford Junior University, Stanford, CA (US)

(72) Inventors: Shane Antony Liddelow, Menlo Park, CA (US); Kevin Guttenplan, Stanford, CA (US); Ben A. Barres, Palo Alto, CA (US)

(73) Assignee: The Board of Trustees of the Leland Stanford Junior University, Stanford, CA (US)

(21) Appl. No.: 16/342,504

(22) PCT Filed: Oct. 25, 2017
(86) PCT No.: PCT/US2017/058261

§ 371 Date: Apr. 16, 2019

Related U.S. Application Data
(60) Provisional application No. 62/413,187, filed on Oct. 26, 2016.

(51) Int. Cl. C07K 16/24; G01N 33/50; C07K 16/18; A61P 25/00

(52) U.S. Cl. CPC C07K 16/245; G01N 33/5058; G01N 33/502; A61K 2039/507; C07K 16/241; A61P 25/00; C07K 16/18

(57) ABSTRACT

Provided are methods of modulating neuronal and/or oligodendrocyte survival. The subject methods relate to preventing neuronal and/or oligodendrocyte death or increasing neuron or oligodendrocyte death. Also provided are neuroprotective compositions and neurotoxic compositions. Methods of identifying neurotoxins, methods of identifying neurotoxin inhibitors and methods of identifying neurotoxin conditions in a subject are also provided.

### CROSS-REFERENCE TO RELATED APPLICATIONS

This application claims priority to the filing date of the U.S. Provisional Patent Application Ser. No. 62/413,187, filed Oct. 26, 2016, the disclosure of which application is herein incorporated by reference in its entirety.

### GOVERNMENT RIGHTS

This invention was made with Government support under contract AG048814 awarded by the National Institutes of Health. The Government has certain rights in the invention.

### BACKGROUND

Astrocytes are abundant cells in the central nervous system (CNS) that provide trophic support for neurons, promote formation and function of synapses, prune synapses by phagocytosis, and maintain the blood-brain barrier, in addition to fulfilling a wide range of other homeostatic maintenance functions. Astrocytes undergo a dramatic transformation called “reactive astrocytosis” after brain injury or disease where they up-regulate many genes, undergo hypertrophy, and form a glial scar after acute CNS injury. The functions of reactive astrocytes have been a subject of some debate, with previous studies showing that they may both hinder or support CNS recovery. It has not been clear under what contexts they may be helpful or harmful and many questions remain about their functions.

Although reactive astrocytes are rapidly generated following brain injuries and neurodegenerative and neuroinflammatory diseases, their role in trauma and disease states is not well understood. Elucidation of the biological roles reactive astrocytes play in influencing neuronal cell type survival is of great interest for the treatment of diseases characterized by neurodegeneration as well as diseases of excess or aberrant neuronal activity. Bypassing, inhibiting or overriding certain functions of reactive astrocytes will provide significant impact by increasing our ability to modulate neuronal survival in a variety of neurological disorders, including those that manifest following CNS injury.

Related publications include: Sofroniew et al., Acta Neuropathol. 119, 7-35 (2010); Clarke et al., Nat. Rev. Neurosci. 14, 311-21 (2013); Chung et al., Nature 504, 394-400 (2013); Liddelow et al., Cell 162, 1170-1170.e1 (2015); Zamanian et al., J. Neurosci. 32, 6391-410 (2012); Anderson et al. Nature 532, 195-200 (2016); Sofroniew et al., Cold Spring Harb Perspect Biol 7, a020420 (2015); Martinez et al., F1000Prime Rep 6, 13 (2014); andHeppner et al., Nat. Rev. Neurosci. 16, 358-72 (2015).

### SUMMARY

Provided are methods of modulating neuronal and/or oligodendrocyte survival. The subject methods relate to preventing neuronal and/or oligodendrocyte death or increasing neuron or oligodendrocyte death. Also provided are neuroprotective compositions and neurotoxic compositions. Methods of identifying neurotoxins, methods of identifying neurotoxin inhibitors and methods of identifying neurotoxin conditions in a subject are also provided.

Aspects of the present disclosure include a method of preventing neuronal or oligodendrocyte death in a subject in need thereof, the method comprising administering to the subject effective amounts of an Interleukin 1 alpha (IL-1α) inhibitor and a tumor necrosis factor alpha (TNFα) inhibitor. In some embodiments the method further comprises administering to the subject an effective amount of a complement component 1, q subcomponent (C1q) inhibitor. In some embodiments the effective amounts synergistically prevent neuronal death. In some embodiments the subject has a neurodegenerative disease. In some embodiments the neurodegenerative disease is Alzheimer's disease, Huntington's disease, Parkinson's disease, amyotrophic lateral sclerosis, Multiple Sclerosis, or an eye-related neurodegenerative disease, such as glaucoma. In some embodiments the subject has a neuroinflammatory disease. In some embodiments the subject has a central nervous system (CNS) injury, such as spinal cord injury (SCI) or stroke. In some embodiments the IL-1α inhibitor directly binds IL-1α. In some embodiments the IL-1α inhibitor is an antibody. In some embodiments the IL-1α inhibitor is a non-antibody IL-1α antagonist. In some embodiments the IL-1α inhibitor is an antagonist of an IL-1α binding protein that prevents binding of IL-1α to the IL-1α binding protein. In some embodiments the TNFα inhibitor directly binds TNFα. In some embodiments the TNFα inhibitor is an antibody. In some embodiments the TNFα inhibitor is a non-antibody TNFα antagonist. In some embodiments the TNFα inhibitor is an antagonist of a TNFα binding protein that prevents binding of TNFα to the TNFα binding protein. In some embodiments the C1q inhibitor directly binds C1q. In some embodiments the C1q inhibitor is an antibody. In some embodiments the C1q inhibitor is a non-antibody C1q antagonist. In some embodiments the C1q inhibitor is an antagonist of a C1q binding protein that prevents binding of C1q to the C1q binding protein. In some embodiments the subject comprises a population of A1 reactive astrocytes at a site of neurotoxicity. In some embodiments the A1 reactive astrocytes of the population express one or more A1 reactive astrocyte markers selected from the group consisting of: H2.T23, Serping1 , H2.D1, Ggta1, ligp1, Gbp2, FbIn5, Ugt1a, Fkbp5, Psmb8, Srgn, Amigo2 and C3. In some embodiments the A1 reactive astrocytes of the population express one or more PAN reactive markers selected from the group consisting of: Lcn2, Steap4, S1pr3, Timp1, Hspb1, Cxcl10 , Cd44, Osmr, Cp, Serpina3n, Aspg, Vim and Gfap. In some embodiments the method further comprises identifying the presence of the population of A1 reactive astrocytes. In some embodiments the identifying comprises detecting the presence of an A1 astrocyte derived neurotoxin in the subject.

Aspects of the present disclosure include a neuroprotective composition comprising an effective amount of an IL-1α inhibitor and a TNFα inhibitor. In some embodiments the composition further comprises a C1q inhibitor. In some embodiments the neuroprotective composition comprises effective amounts that synergistically prevent neuronal death, oligodendrocyte death or a combination thereof. In some embodiments the neuroprotective composition is in unit dosage form.

Aspects of the present disclosure include a method of identifying an inhibitor of a neurotoxin, the method comprising: culturing a neuron or oligodendrocyte in a medium conditioned with an A1 reactive astrocyte that produces the neurotoxin; contacting the cultured neuron or oligodendrocyte with a candidate inhibitor; assaying the neuron or oligodendrocyte for viability, wherein when the neuron or oligodendrocyte has increased viability as compared to a control neuron or oligodendrocyte the candidate inhibitor is identified as an inhibitor of the neurotoxin. In some embodiments the method further includes generating the A1 reactive astrocyte by contacting an astrocyte or a progenitor thereof with IL-1α, TNFα and C1q. In some embodiments the control neuron or oligodendrocyte is cultured in the medium but not contacted with the candidate inhibitor. In some embodiments the A1 reactive astrocyte expresses one or more A1 reactive astrocyte markers selected from the group consisting of: H2.T23, Serping1 , H2.D1, Ggta1, ligp1, Gbp2, FbIn5, Ugt1a, Fkbp5, Psmb8, Srgn, Amigo2 and C3. In some embodiments the A1 reactive astrocyte express one or more PAN reactive markers selected from the group consisting of: Lcn2, Steap4, S1pr3, Timp1, Hspb1, Cxcl10, Cd44, Osmr, Cp, Serpina3n, Aspg, Vim and Gfap. In some embodiments the neuron is a central nervous system (CNS) neuron. In some embodiments the CNS neuron is selected from the group consisting of: a cortical neuron, a spinal motor neuron and a retinal ganglion cell. In some embodiments the neuron or oligodendrocyte is a mammalian neuron or oligodendrocyte. In some embodiments the neurotoxin is heat sensitive. In some embodiments the neurotoxin is protease sensitive. In some embodiments the neurotoxin is greater than 30 kD in size.

Aspects of the present disclosure include a method of identifying a neurotoxin, the method comprising: generating a medium conditioned with an A1 reactive astrocyte that produces the neurotoxin; purifying the neurotoxin from the conditioned medium; and identifying the purified neurotoxin. In some embodiments the identifying comprises mass spectrometry. In some embodiments the purifying comprises fractionating the conditioned medium into media fractions. In some embodiments the method comprises assaying the media fractions for neuronal or oligodendrocyte cell killing. In some embodiments the method further comprises assaying the purified neurotoxin for neuronal or oligodendrocyte cell killing.

Aspects of the present disclosure include a neurotoxic composition comprising the neurotoxin identified according to any of the methods described herein. Aspects of the present disclosure include a method of killing a neuron or oligodendrocyte, the method comprising contacting the neuron or oligodendrocyte with such a composition.

Aspects of the present disclosure include a method of identifying a neurotoxic condition in a subject, the method comprising: detecting the level of a neurotoxin identified according to a method described herein in a sample obtained from the subject; and identifying the subject as having a neurotoxic condition when the detected level of the neurotoxin is above a reference level. In some embodiments the sample comprises cerebrospinal fluid. In some embodiments the sample comprises blood. In some embodiments the reference level is based on the level of the neurotoxin present in a normal sample. In some embodiments the method further comprises treating the subject for the neurotoxic condition when the subject is identified as having a neurotoxic condition. In some embodiments the treating comprises a method of preventing neuronal or oligodendrocyte death as described herein.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1H demonstrate a serum-free culture model for A1 reactive astrocytes.

FIG. 2A-2G demonstrate that A1 reactive astrocytes do not promote synapse formation or function.

FIG. 3A-3G demonstrate that A1 astrocytes lose phagocytic capacity.

FIG. 4A-4O demonstrate the effects of an astrocyte-derived toxic factor promoting cell death.

FIG. 5A-5V demonstrate the presence of A1 reactive astrocytes in human disease FIG. 6 demonstrates that the inhibition of IL-1α, TNFα and C1q signaling prevents A1 reactive astrocyte formation and increases neuron survival.

FIG. 7A-7E provide the results of FACS analysis of Csf1r−/−mice.

FIG. 8A-8C provide a schematic of and the results of a screen for A1 reactive mediators as described herein.

FIG. 9A-9F provide further results of a screen for A1 reactive mediators as described herein.

FIG. 10A-10G demonstrate that A1 astrocytes are morphologically simple.

FIG. 11A-11F demonstrate that A1 reactive astrocytes do not promote synapse formation or neurite outgrowth.

FIG. 12 demonstrates that P4 lateral geniculate nucleus astrocytes become A1 reactive following systemic LPS injection.

FIG. 13A-13R demonstrate various effects of an astrocyte-derived toxic factor promoting cell death.

FIG. 14A-14L demonstrate the pharmacological blockade of an astrocyte-derived toxic factor promoting cell death.

FIG. 15A-15K demonstrate that A1 reactive astrocytes inhibit oligodendrocyte precursor cell differentiation and migration.

FIG. 16A-16C provide single cell analysis of C3 expression following neuroinflammatory and ischemic injury.

FIG. 17A-17I demonstrate that blocking formation of A1 reactive astrocytes preserves neuronal health in a mouse model of glaucoma.

FIG. 18A-18B provide gene expression heat maps showing upregulation of astrocyte reactive transcripts in bead injected eyes and a general lack of such transcripts in injected eyes of Il1α−/−Tnf−/−C1qa−/− animals.

FIG. 19 demonstrates that retinal ganglion cell killing in an optic nerve crush model was dependent on neuronal injury and the presence of secreted factors from A1 astrocytes.

FIG. 20A-20C show astrocyte activation close to the lesion site following injury in a weight-drop model of spinal cord injury (SCI).

FIG. 21 shows astrocyte activation in the hindbrain (right) and the cortex (left) following injury in a weight-drop model of SCI.

FIG. 22 demonstrates that blocking formation of A1 reactive astrocytes results in an early decrease in infarct size following stroke.

FIG. 23 provides a comparison of GFAP+ cell density at an early and late timepoint following stroke in wildtype and Il1α−/−Tnf−/−C1qa−/− animals.

### DEFINITIONS

The terms “A1 reactive astrocytes” and “A1 astrocytes” are used interchangeably herein and generally refer to a subclass of astrocytes that are a non-resting astrocyte population. A1 astrocytes are distinguished from other astrocyte populations, e.g., resting astrocytes, A2 reactive astrocytes, etc., in various ways including at least in part by e.g., the kind of inducing event (e.g., the kind of inducing injury), gene expression (e.g., gene expression profiles) and their influence(s) on non-astrocyte cell populations (e.g., neurons, oligodendrocytes, etc.). The present disclosure describes that A1 reactive astrocytes are harmful (i.e., detrimental to neuronal and/or oligodendrocyte viability) and are induced by classically-activated neuroinflammatory microglia. A1 reactive astrocytes may be defined and/or identified based on gene expression, including e.g., based on the expression of one or more A1 reactive astrocyte markers including but not limited to e.g., H2.T23, Serping1, H2.D1, Ggta1, ligp1, Gbp2, Fbln5 , Ugt1a, Fkbp5, Psmb8, Srgn, Amigo2 and C3. A1 reactive astrocytes will also generally express or overexpress (e.g., as compared to resting astrocytes) one or more ‘pan reactive’ genes (i.e., genes having expression associated with reactive astrocytes of various subgroups). Pan reactive genes include but are not limited to e.g., Lcn2, Steap4, S1pr3, Timp1, Hspb1, Cxcl10 , Cd44, Osmr, Cp, Serpina3n, Aspg, Vim and Gfap. A1 reactive astrocytes may also lack expression or not demonstrate overexpression of one or more A2 reactive astrocyte associated genes including but not limited to e.g., Clcf1, Tgm1, Ptx3, S100a10, Sphk1, Cd109, Ptgs2 , Emp1, Slc10a6, Tm4sf1, B3gnt5 and Cd14.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the identity of” includes determining the most likely identity of a particular compound or formulation or substance, and/or determining whether a predicted compound or formulation or substance is present or absent.

The term “bodily fluid” as used herein generally refers to fluids derived from a “biological sample” which encompasses a variety of sample types obtained from an individual or a population of individuals and can be used in a diagnostic, monitoring or screening assay. The definition encompasses blood and other liquid samples of biological origin. The definition also includes samples that have been manipulated in any way after their procurement, such as by mixing or pooling of individual samples, treatment with reagents, solubilization, or enrichment for certain components, such as nucleated cells, non-nucleated cells, pathogens, etc.

The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like.

The terms “control”, “control assay”, “control sample” and the like, refer to a sample, test, or other portion of an experimental or diagnostic procedure or experimental design for which an expected result is known with high certainty, e.g., in order to indicate whether the results obtained from associated experimental samples are reliable, indicate to what degree of confidence associated experimental results indicate a true result, and/or to allow for the calibration of experimental results. For example, in some instances, a control may be a “negative control” assay such that an essential component of the assay is excluded such that an experimenter may have high certainty that the negative control assay will not produce a positive result. In some instances, a control may be “positive control” such that all components of a particular assay are characterized and known, when combined, to produce a particular result in the assay being performed such that an experimenter may have high certainty that the positive control assay will not produce a positive result. Controls may also include “blank” samples, “standard” samples (e.g., “gold standard” samples), validated samples, etc.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human. In some cases, the methods of the invention find use in experimental animals, in veterinary application, and in the development of animal models, including, but not limited to, rodents including mice, rats, and hamsters; and primates.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. For example, a preventative treatment, i.e. a prophylactic treatment, may include a treatment that effectively prevents a condition (e.g., a neurodegenerative condition) or a treatment that effectively prevents or controls progression of a condition (e.g., a neurodegenerative condition). In some instances, the treatment may result in a treatment response, such as a complete response or a partial response. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s).

Those in need of treatment can include those already afflicted (e.g., those with a central nervous system (CNS) injury (e.g., acute CNS injury, chronic CNS injury, etc.), those with neurodegeneration, those with neuroinflammation, etc.) as well as those in which prevention is desired (e.g., those with increased susceptibility to CNS injury, neurodegeneration, or neuroinflammation; those suspected of having CNS injury, neurodegeneration, or neuroinflammation; those with an increased risk of developing CNS injury, neurodegeneration, or neuroinflammation; those with increased environmental exposure to practices or agents causing CNS injury, neurodegeneration, or neuroinflammation, those suspected of having a genetic or behavioral predisposition to CNS injury, neurodegeneration, or neuroinflammation; those with CNS injury, neurodegeneration, or neuroinflammation, those having results from screening indicating an increased risk of CNS injury, neurodegeneration, or neuroinflammation, those having tested positive for a CNS injury, neurodegeneration, or neuroinflammation related condition; those having tested positive for one or more biomarkers of a CNS injury, neurodegeneration, or neuroinflammation related condition, etc.).

A therapeutic treatment is one in which the subject is afflicted prior to administration and a prophylactic treatment is one in which the subject is not afflicted prior to administration. In some embodiments, the subject has an increased likelihood of becoming afflicted or is suspected of having an increased likelihood of becoming afflicted (e.g., relative to a standard, e.g., relative to the average individual, e.g., a subject may have a genetic predisposition to a neurological condition and/or a family history indicating increased risk of neurodegeneration or neuroinflammation), in which case the treatment can be a prophylactic treatment.

As used herein, the terms “inhibit” and “block” are used interchangeably and refer to the function of a particular agent to effectively impede, retard, arrest, suppress, prevent, decrease, or limit the function or action of another agent or agents or cell or cells or cellular process or cellular processes. In such instances an agent that inhibits is referred to as an “inhibitor”, which term is used interchangeably with “inhibitory agent” and “antagonist”. As used herein, the term “inhibitor” refers to any substance or agent that interferes with or slows or stops a chemical reaction, a signaling reaction, or other biological or physiological activity. An inhibitor may be a direct inhibitor that directly binds the substance or a portion of the substance that it inhibits or it may be an indirect inhibitor that inhibits through an intermediate function, e.g., through binding of the inhibitor to an intermediate substance or agent that subsequently inhibits a target.

As used herein the term “small molecule” refers to a small organic or inorganic compound having a molecular weight of more than 50 and less than about 2,500 daltons. Agents may comprise functional groups necessary for structural interaction with proteins, particularly hydrogen bonding, and may include at least an amine, carbonyl, hydroxyl or carboxyl group, and may contain at least two of the functional chemical groups. The small molecule agents may comprise cyclical carbon or heterocyclic structures and/or aromatic or polyaromatic structures substituted with one or more functional groups. Small molecule agents are also found among biomolecules including peptides, saccharides, fatty acids, steroids, purines, pyrimidines, derivatives, structural analogs or combinations thereof.

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

The terms “short interfering RNA”, “siRNA”, and “short interfering nucleic acid” are used interchangeably may refer to short hairpin RNA (shRNA), short interfering oligonucleotide, short interfering nucleic acid, short interfering modified oligonucleotide, chemically-modified siRNA, post-transcriptional gene silencing RNA (ptgsRNA), and other short oligonucleotides useful in mediating an RNAi response. In some instances siRNA may be encoded from DNA comprising a siRNA sequence in vitro or in vivo as described herein. When a particular siRNA is described herein, it will be clear to the ordinary skilled artisan as to where and when a different but equivalently effective interfering nucleic acid may be substituted, e.g., the substation of an short interfering oligonucleotide for a described shRNA and the like.

The terms “pluripotent progenitor cells”, “pluripotent progenitors”, “pluripotent stem cells”, “multipotent progenitor cells” and the like, as used herein refer to cells that are capable of differentiating into two or more different cell types and proliferating. Non limiting examples of pluripotent precursor cells include but are not limited to embryonic stem cells, blastocyst derived stem cells, fetal stem cells, induced pluripotent stem cells, ectodermal derived stem cells, endodermal derived stem cells, mesodermal derived stem cells, neural crest cells, amniotic stem cells, cord blood stem cells, adult or somatic stem cells, neural stem cells, bone marrow stem cells, bone marrow stromal stem cells, hematopoietic stem cells, lymphoid progenitor cell, myeloid progenitor cell, mesenchymal stem cells, epithelial stem cells, adipose derived stem cells, skeletal muscle stem cells, muscle satellite cells, side population cells, intestinal stem cells, pancreatic stem cells, liver stem cells, hepatocyte stem cells, endothelial progenitor cells, hemangioblasts, gonadal stem cells, germline stem cells, and the like. Pluripotent progenitor cells may be acquired from public or commercial sources or may be newly derived. As described herein, in some instances, pluripotent progenitor cells of the subject disclosure are those cells capable of giving rise to neuronal cell types or derivatives (e.g., neurons), oligodendrocyte precursors or derivatives (e.g., oligodendrocytes), astrocyte precursors or derivatives (e.g., astrocytes), and the like. Pluripotent progenitors not naturally having the capacity to generate neuronal cell types or derivatives thereof, oligodendrocyte precursors or derivatives thereof, astrocyte precursors or derivatives thereof, may be dedifferentiated to a cell type having such capacity by methods well-known in the art, including, e.g., those methods for the production of induced pluripotent cells. For example, a cell may be naturally capable of giving rise to desired cell type(s) or may be artificially made (e.g., reprogrammed, dedifferentiated, transdifferentiated, etc.) to be capable of giving rise to desired cell type(s). By “naturally capable” is meant that giving rise to desired cell type(s) represents part of the natural developmental lineage or the natural differentiation potential of the cell. As such, cells artificially made capable of giving rise to particular desired cell type(s) are generally cells that do not have such capability naturally.

### DETAILED DESCRIPTION

Provided are methods of modulating neuronal and/or oligodendrocyte survival. The subject methods relate to preventing neuronal and/or oligodendrocyte death or increasing neuron or oligodendrocyte death. Also provided are neuroprotective compositions and neurotoxic compositions. Methods of identifying neurotoxins, methods of identifying neurotoxin inhibitors and methods of identifying neurotoxin conditions in a subject are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

As summarized above, the present disclosure provides methods of modulating neuronal and/or oligodendrocyte cell survival. By “modulating cell survival” is meant increasing or decreasing the viability of one or more cells by administering one or more agents and/or contacting the cells with a composition, i.e., a neuroprotective composition or a neurotoxic composition. Modulating cell survival may include where, e.g., the viability of the cells is increased or decreased as compared to the viability of control cells, i.e., cells not contacted with a neuroprotective or neurotoxic composition including e.g., those neuroprotective or neurotoxic compositions described herein.

Modulating the survival of a cell to increase survival may include preventing neuronal and/or oligodendrocyte death. As described in more detail below, methods of preventing neuronal and/or oligodendrocyte death may include contacting a neuronal and/or oligodendrocyte cell with an interleukin 1 alpha (IL-1α) inhibitor and a tumor necrosis factor alpha (TNFα) inhibitor. Such contacting will vary, as described in more detail below, and may include administering an IL-1α inhibitor and a TNFα inhibitor to a subject within which the neuronal and/or oligodendrocyte cell is present. Methods of preventing neuronal and/or oligodendrocyte death may also include contacting a neuronal and/or oligodendrocyte cell with an IL-1α inhibitor, a TNFα inhibitor and a complement component 1, q subcomponent (C1q) inhibitor. Such contacting will vary, as described in more detail below, and may include administering an IL-1α inhibitor, a TNFα inhibitor and a C1q inhibitor to a subject within which the neuronal and/or oligodendrocyte cell is present.

Modulating the survival of a cell to decrease survival may include inducing neuronal and/or oligodendrocyte death, i.e., killing neuronal and/or oligodendrocyte cells. As described in more detail below, methods of killing neuronal and/or oligodendrocyte cells may include contacting a neuronal and/or oligodendrocyte cell with a neurotoxin, including e.g., where such neurotoxin is present in a composition, just as e.g., a conditioned media. Such contacting will vary, as described in more detail below, and may include locally administering the neurotoxin to a subject at a location within which the neuronal and/or oligodendrocyte cell is present.

The present methods are directed to modulating the survival of neuronal cells and/or oligodendrocyte cells. By “neuronal cells” is generally meant any neuron. In some instances, the methods may modulate the survival of central nervous system (CNS) neurons, where such CNS neurons will vary and may include but are not limited to e.g., cortical neurons, spinal neurons, retinal ganglion cells, cranial nerves, brainstem neurons, cerebellum neurons, diencephalon neurons, cerebrum neurons, and the like. By “oligodendrocytes” is generally meant those cells that are a subset of neuroglia that develop from oligodendrocyte precursor cells (OPCs) and provide a primary function in myelination axons of the central nervous system any may be identified by a variety of markers including but not limited to e.g., GD3, NG2 chondroitin sulfate proteoglycan, platelet-derived growth factor-alpha receptor subunit (PDGF-alphaR), and the like. Oligodendrocytes, the survival of which may be modulated according to the herein described methods, may vary and may include immature and mature oligodendrocytes, where, as described herein, mature oligodendrocytes may be more susceptible to A1 reactive astrocyte neurotoxins as compared to immature oligodendrocytes. In some instances, a neuron or oligodendrocyte of the instant methods may express a receptor for a neurotoxin, or may have increased susceptibility to a neurotoxin (e.g., through injury to the neuron), and the like, e.g., as described in more detail below.

Neurons and/or oligodendrocytes of the instant methods may be derived from a variety of different animals including e.g., mammals including but not limited to e.g., humans, horses, pigs, sheep, goats, dogs, cats, rats, mice, and the like.

The methods of the present disclosure are based, at least in part, on the discovery that a particular population of astrocytes, termed ‘A1 reactive astrocytes’, produce and secrete a neurotoxin that effectively kills neurons and oligodendrocytes. It was also discovered that A1 reactive astrocytes are produced by activating IL-1α and TNFα signaling in astrocytes, e.g., by contacting non-reactive astrocytes (i.e., resting astrocytes) or astrocyte precursors with IL-1α and TNFα. Furthermore, it was found that signaling through complement component 1, q subcomponent (C1q), e.g., by contacting non-reactive astrocytes or astrocyte precursors with C1q, when combined with increased IL-1α and TNFα signaling increases the production of A1 reactive astrocytes. Upon induction to A1 reactive astrocyte fate by being contacted with IL-1α and TNFα or IL-1α, TNFα and C1q, A1 reactive astrocytes secrete a neurotoxin that triggers neuronal and oligodendrocyte death.

A1 reactive astrocytes may be characterized in various ways. For example, in some instances, A1 reactive astrocytes are characterized in that they become neurotoxic upon activation of IL-1α and TNFα or IL-1α, TNFα and C1q signaling. A1 reactive astrocytes may also be characterized based on the expression of one or more A1 reactive astrocyte markers. Such markers will vary and may include but are not limited to e.g., H2.T23, Serping1, H2.D1, Ggta1 , ligp1, Gbp2, Fbln5, Ugt1a, Fkbp5, Psmb8, Srgn, Amigo2, C3 and combinations thereof. In some instances, a A1 reactive astrocyte may be characterized as expressing two or more A1 reactive astrocyte markers including but not limited to e.g., 3 or more, 4 or more, 5 or more, 6 or more, 7 or more, 8 or more, 9 or more, 10 or more, etc., A1 reactive astrocyte markers.

A1 reactive astrocytes may be characterized based on expression of one or more PAN reactive (i.e., pan reactive astrocyte) markers in addition to the one or more A1 reactive astrocyte markers described above. PAN reactive astrocyte markers will vary and may include but are not limited to e.g., Lcn2, Steap4, S1pr3, Timp1, Hspb1, Cxcl10, Cd44, Osmr, Cp, Serpina3n, Aspg, Vim and Gfap. In some instances, a A1 reactive astrocyte may be characterized as expressing two or more PAN reactive markers including but not limited to e.g., 3 or more, 4 or more, 5 or more, 6 or more, 7 or more, 8 or more, 9 or more, 10 or more, etc., PAN reactive markers.

A1 reactive astrocytes may be characterized based on a lack expression or a lack of overexpression of one or more A2 reactive astrocyte associated genes. A2 reactive astrocyte associated genes will vary and may include but are not limited to e.g., Clcf1, Tgm1, Ptx3 , S100a10, Sphk1, Cd109, Ptgs2, Emp1, Sic10a6, Tm4sf1, B3gnt5 and Cd14. In some instances, a A1 reactive astrocyte may be characterized as not expressing (i.e., being “negative for”) or not overexpressing two or more A2 reactive astrocyte associated genes including but not limited to e.g., 3 or more, 4 or more, 5 or more, 6 or more, 7 or more, 8 or more, 9 or more, 10 or more, etc., A2 reactive astrocyte associated genes.

The expression A1 reactive astrocyte markers may be detected and/or measured in various ways including direct and indirect methods of both detection and quantification. Expression of A1 reactive astrocyte makers may be performed on a representative sample of cells which all or a portion of may be predicted or suspected of being A1 reactive astrocytes. The expression of one or more A1 reactive astrocyte markers may also be detected and/or measured by obtaining a sample of one or more cells of a population of cells of interest and directly or indirectly assessing the gene expression of the sampled cells. In some instances, the sample of cells may be obtained from a specimen taken from a subject, e.g., a biopsy or other biological sample.

In some instances, the expression of one or more markers, e.g., A1 reactive astrocyte markers, PAN reactive astrocyte markers, A2 reactive astrocyte makers, etc., may be detected and/or measured by contacting a cell of interest with a labeled probe that is specific for a nucleic acid encoding the marker or a labeled binding agent specific for the marker itself. According to such methods, following contacting with a labeled probe or labeled specific binding member, the amount of labeling of the subject cell may be assessed, e.g., as compared to the amount of labeling present in a known A1 reactive astrocyte and/or the amount of labeling in a related cell that is known not to be an A1 reactive astrocyte, in order to identify whether the cell is or is not an A1 reactive astrocyte. The expression of one or more cells labeled with a labeled probe or specific binding member may be assessed by any convenient and appropriate means including but not limited to e.g., cytometric methods (including e.g., cell cytometry, image cytometry, flow cytometry, etc.), microscopic methods (e.g., fluorescent microscopy, etc.), and the like.

In some instances, a sample of cells may be collected and the expression level(s) of one or more markers, e.g., A1 reactive astrocyte markers, may be measured by a quantitative gene expression assay. Quantitative gene expression assays will vary and may include but are not limited to e.g., quantitative PCR, microarray, quantitative sequencing, etc. In some instances, the amount of expression of one or more markers may be compared to a reference expression level for the one or more markers. Reference expression levels may be derived from any convenient and appropriate source including but not limited to e.g., the level of the marker(s) expressed in cells known not to be A1 reactive astrocytes, the level of the marker(s) expressed in cells known to be A1 reactive astrocytes.

In some instances, whether a subject cell is an A1 reactive astrocyte may be assessed based on expression of one or more reporter constructs specific for a marker, e.g., an A1 reactive astrocyte marker. Such reporter constructs will vary and may include but are not limited to e.g., an A1 astrocyte marker promoter (e.g., an endogenous promoter or a heterologous promoter) operably linked to a sequence encoding a detectable polypeptide (e.g., a fluorescent protein, a colorimetric protein, a polypeptide or peptide tag, an enzyme for a detectable substrate (e.g., horseradish peroxidase, alkaline phosphatase, etc.), and the like). Upon induction of the promoter the reporter may be expressed allowing detection and/or quantification of the reporter indicating expression of the A1 reactive astrocyte marker. In some instances, the amount of expression of one or more A1 reactive astrocyte markers measured using a reporter construct may be compared to a reference expression level for the one or more markers using the same or comparable reporter constructs. Reference expression levels may be derived from any convenient and appropriate source including but not limited to e.g., the level of the marker(s) expressed in cells known not to be A1 reactive astrocytes, the level of the marker(s) expressed in cells known to be A1 reactive astrocytes.

Methods of the present disclosure may include generating A1 reactive astrocytes, e.g., by contacting an astrocyte or a progenitor thereof with IL-1α and TNFα or IL-1α, TNFα and C1q. In some instances, an A1 reactive astrocyte may be generated from an astrocyte that is not an A1 reactive astrocyte including but not limited to e.g., a non-reactive astrocyte (i.e., a resting astrocyte), an astrocyte progenitor. Generated A1 reactive astrocytes may include cells (e.g., astrocyte progenitors, non-reactive astrocytes, etc.) induced to express one or more A1 reactive astrocyte markers, including e.g., those described above. A1 reactive astrocytes, whether naturally or artificially (i.e., synthetically) produced, may be cultured in a culture medium to generate an A1 reactive astrocyte conditioned culture medium. Described in more detail below, an A1 reactive astrocyte conditioned culture medium will include substances secreted by A1 reactive astrocytes including but not limited to e.g., an A1 reactive astrocyte secreted neurotoxin.

### Preventing Neuronal or Oligodendrocyte Death

As summarized above, the present disclosure includes methods of preventing neuronal and/or oligodendrocyte death e.g., by inhibiting the generation of A1 reactive astrocytes and/or the production and/or action of a neurotoxin produced by A1 reactive astrocytes. In some instances, increasing neuronal and/or oligodendrocyte survival involves administering to a subject effective amounts of an IL-1α inhibitor and a TNFα inhibitor. In some instances, the effective amounts of IL-1α inhibitor and TNFα inhibitor synergistically increase neuron and/or oligodendrocyte viability. In some instances, increasing neuronal and/or oligodendrocyte survival involves administering to a subject effective amounts of an IL-1α inhibitor, a TNFα inhibitor and a C1q inhibitor. In some instances, the effective amounts of IL-1α inhibitor, TNFα inhibitor and C1q inhibitor synergistically increase neuron and/or oligodendrocyte viability. In some instances, IL-1α inhibitor and TNFα inhibitor or IL-1α inhibitor, TNFα inhibitor and C1q inhibitor are administered to a subject in need thereof, e.g., a subject in need of preventing neuronal and/or oligodendrocyte cell death.

Subjects of the present methods will vary and may include but are not limited to e.g., subjects suspected of having increased levels of neuronal cell death, subjects suspected of having increased levels of oligodendrocyte death, subjects suspected of having increased levels of neuronal and oligodendrocyte cell death, subjects known to have increased levels of neuronal cell death, subjects known to have increased levels of oligodendrocyte death, subjects known to have increased levels of neuronal and oligodendrocyte cell death, subjects suspected of having or known to have increased levels of A1 reactive astrocytes, and the like.

In some instances, subjects of the present methods include subjects that do not currently have increased levels of neuronal and/or oligodendrocyte cell death but will be subjected to or otherwise exposed to conditions predicted to cause neuronal and/or oligodendrocyte death. As such, in some instances, the present methods include preventing neuron and/or oligodendrocyte cell death in a subject that does not have increased levels of neuronal and/or oligodendrocyte cell death but may, e.g., be expected to be exposed to conditions that increase neuronal and/or oligodendrocyte cell death.

Subjects of the present methods may include but are not limited to e.g., subjects having conditions characterized by increased levels of neuronal cell death, increased levels of oligodendrocyte death, increased levels of A1 astrocytes, or combinations thereof. Such conditions include but are not limited to e.g., conditions involving CNS injury. As used herein, the term “CNS injury condition” generally includes any condition, acute or chronic, involving the death or degeneration of one or more CNS neuronal cell types or cells associated with CNS neuronal cell types that directly support the survival and/or proper functioning of one or more CNS neurons. Non-limiting examples of CNS injury conditions include but are not limited to e.g., traumatic CNS injury (e.g., traumatic brain injury (TBI) (e.g., severe TBI, moderate brain injury, mild TBI (MTBI, i.e. concussion)), spinal cord injury (SCI), traumatic injury to the eye (including traumatic injury to the nerves of the eye, such as the optic nerve), ischemia, CNS stroke, neurodegenerative disease, neuroinflammatory disease, and the like.

In some instances, a subject in need of preventing neuronal and/or oligodendrocyte death may be a subject having suffered traumatic CNS injury (i.e., CNS neurotrauma). Areas of the CNS that may be injured in a CNS injury include but are not limited to e.g., brain, the spine, etc., as well as neural projections to/from the CNS such as e.g., optic nerves and the like. Non-limiting examples of CNS injuries include traumatic brain injury (TBI), traumatic spinal cord injury (SCI), CNS crush injuries, CNS injuries resulting from a neoplasia (e.g., a brain cancer, e.g., brain tumor), and the like. As used herein, the term CNS injury encompasses injury that occurs as a result of a CNS stroke (e.g., infarct).

In some instances, a subject in need of preventing neuronal and/or oligodendrocyte death may be a subject having suffered a CNS stroke or a subject at increased risk of developing a CNS stroke. The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardic arrest, cardiogenic shock, septic shock, head trauma, spinal cord trauma, seizure, bleeding from a tumor, or other blood loss.

Risk factors for stroke include but are not limited to e.g., high blood pressure, diabetes, heart disease, smoking, increased age (e.g., over 65 years), oral contraceptive use, African America descent, Alaskan Native descent, American Indian descent, family history of stroke, family history of transient ischemic attack (TIA), personal history of stroke, personal history of transient ischemic attack (TIA), brain aneurysm, arteriovenous malformation (AVM), Alcohol and illegal drug use (e.g., cocaine, amphetamines), sickle cell disease, vasculitis (inflammation of the blood vessels), bleeding disorders, overweight, obesity, stress, depression, unhealthy cholesterol levels, use of nonsteroidal anti-inflammatory drugs (NSAIDs) excluding aspirin, and combinations thereof.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow. Cerebral ischemic episodes result from a deficiency in the blood supply to the brain. The spinal cord, which is also a part of the central nervous system, is equally susceptible to ischemia resulting from diminished blood flow.

In some instances, a subject in need of preventing neuronal and/or oligodendrocyte death may be a subject having a neurodegenerative disease or a subject at increased risk of developing a neurodegenerative disease. Non-limiting examples of neurodegenerative diseases include Alzheimer's disease, Huntington's disease, Parkinson's disease, amyotrophic lateral sclerosis, Multiple Sclerosis, Motor neurone diseases (MND), Spinocerebellar ataxia (SCA), Spinal muscular atrophy (SMA), eye-related neurodegenerative disease (e.g., glaucoma, diabetic retinopathy, age-related macular degeneration (AMD), etc.), and the like.

In some instances, a subject in need of preventing neuronal and/or oligodendrocyte death may be a subject having or at risk of having glaucoma. Such a subject may display one or more symptoms of glaucoma or risk factors for glaucoma including but not limited to e.g., ocular hypertension, above normal ocular pressure (eye pressure of greater than 22 mm Hg), change in vision (including loss of vision), hazy vision, blurred vision, appearance of rainbow-colored circles around bright lights, severe eye pain, head pain, nausea/vomiting accompanying severe eye pain, African American descent, Hispanic descent, Asian descent, Japanese descent, age over 60 years, family history of glaucoma, steroid use, eye injury, high myopia (nearsightedness), hypertension, central corneal thickness less than 0.5 mm, and combinations thereof.

In some instances, a subject in need of preventing neuronal and/or oligodendrocyte death may be a subject having a neuroinflammatory disease or a subject at increased risk of developing a neuroinflammatory disease. Non-limiting examples of neuroinflammatory diseases include Acute disseminated encephalomyelitis (ADEM), Optic Neuritis (ON), Transverse Myelitis, Neuromyelitis Optica (NMO) and the like. In some instances, primary conditions with secondary neuroinflammation (e.g., traumatic brain injury with secondary neuroinflammation) may be considered a neuroinflammatory disease as it relates to the subject disclosure.

In some instances, a subject in need of preventing neuronal and/or oligodendrocyte death may be a subject having a population of A1 reactive astrocytes at a site of neurotoxicity. The presence of A1 reactive astrocytes at a site of neurotoxicity may be confirmed, e.g., by assaying (including e.g., detecting and/or measuring) for the presence of A1 reactive astrocytes at the site, or may be inferred e.g., from the presence of one or more clinical symptoms indicative of the presence of A1 reactive astrocytes or the presence of an injury commonly associated with an increase in the presence of A1 reactive astrocytes at the site. By “site of neurotoxicity” is generally meant any site associated with the death of neurons, e.g., CNS neurons, and/or oligodendrocytes and may include e.g., any site commonly associated with one or more CNS injury conditions, including e.g., those described above. Non-limiting examples of sites of neurotoxicity may include but are not limited to e.g., sites of neuronal injury (e.g., sites of brain injury, sites of spinal cord injury, and the like), sites associated with a CNS stroke (i.e., a site in the CNS adjacent to or within the affected area of a CNS stroke), sites of neurodegeneration, sites of neuroinflammation, and the like.

As noted above, in some instances, a subject in need of preventing neuronal and/or oligodendrocyte death may be a subject having a population of A1 reactive astrocytes, e.g., as detected by assaying (including e.g., detecting and/or measuring) for the presence of A1 reactive astrocytes at the site. Accordingly, in some instances, methods of the present disclosure may include identifying the presence of A1 reactive astrocytes (i.e., a population of A1 reactive astrocytes) in the subject. A1 reactive astrocytes in a subject may be directly detected of indirectly detected. For example, the presence of A1 reactive astrocytes may be directly detected by detecting one or more cells expressing one or more A1 reactive astrocytes markers, including but not limited to e.g., H2.T23, Serping1, H2.D1, Ggta1, ligp1, Gbp2, Fbn5 , Ugt1a, Fkbp5, Psmb8, Srgn, Amigo2, C3 and combinations thereof. In some instances, the presence of A1 reactive astrocytes may be indirectly detected by detecting one or more secreted factors indicative of the presence of A1 reactive astrocyte markers including but not limited to e.g., an A1 reactive astrocyte secreted neurotoxin, in a sample obtained from the subject.

The present methods include contacting a neuron and/or an oligodendrocyte with, e.g., by administering to a subject, an IL-1α inhibitor. IL-1α inhibitors will vary and may include agents that inactivate or otherwise prevent IL-1α signaling, e.g., by directly binding IL-1α and/or by preventing IL-1α from binding its receptor (e.g., by binding Interleukin 1 receptor, type I (IL1R1) also known as CD121a (Cluster of Differentiation 121a)) in a manner that prevents IL-1α binding and/or signaling) or by preventing the expression of IL-1α.

IL-1α (also known as hematopoietin 1) and IL-1α signaling are well understood in the art and described in, e.g., Di Paolo & Shayakhmetov. Nat Immunol. (2016) 17(8):906-13, the disclosure of which is incorporated herein by reference. Briefly, IL-1α is processed by the removal of N-terminal amino acids by specific proteases to produce the mature form. Both the 31 kDa precursor form of IL-1α and its 18 kDa mature form are biologically active. The three-dimensional structure of the IL-1α contains an open-ended barrel composed entirely of beta-pleated strands and crystal structure analysis shows that it has two sites of binding to IL-1 receptor with a primary binding site located at the open top of its barrel. IL-1 stimulates thymocyte proliferation by inducing IL-2 release, B-cell maturation and proliferation, and fibroblast growth factor activity. IL-1 proteins are involved in the inflammatory response, being identified as endogenous pyrogens.

In some instances IL-1α inhibitory agents (i.e., IL-1α inhibitors) are agents that directly bind IL-1α. IL-1α inhibitory agents that directly bind to IL-1α may inhibit various functions of IL-1α including, but not limited to, binding of IL-1α to an IL-1α receptor, binding of IL-1α processing agents thus inhibiting processing of IL-1α, and the like. In other instances, IL-1α inhibitory agents are agents that directly bind IL-1α may prevent IL-1α from being expressed, e.g., by preventing newly translated IL-1α from being transported to the cell membrane or by preventing modification of IL-1α that allows IL-1α to be expressed. In some instances, IL-1α inhibitor agents may prevent the release of IL-1α from an IL-1α-releasing cell (e.g., an A1 reactive astrocyte). For example, in addition to other methods of preventing release, inhibiting the production or expression of IL-1α may in turn prevent its release.

In some instances IL-1α inhibitory agents are agents that directly bind an IL-1α receptor and antagonize binding of IL-1α to a IL-1α receptor. Binding of an IL-1α inhibitory agent to an IL-1α receptor may block IL-1α signaling through means other than preventing IL-1α from binding its receptor including, e.g., preventing signal transduction.

In some instances a IL-1α inhibitory agent may decrease the effective concentration of soluble IL-1α. For example, in some instances a IL-1α inhibitory agent may be a soluble form of or a solubilized portion of a IL-1α receptor. Such agents that decrease the effective concentration of soluble IL-1α bind or sequester soluble IL-1α without activating IL-1α signaling thus decreasing the amount of free soluble IL-1α available to bind IL-1α receptors capable of activating IL-1α signaling.

In some instances a IL-1α inhibitory agent may be an antibody or fragment thereof that directly binds to IL-1α or a IL-1α receptor, including but not limited to, e.g., an isolated antibody, a recombinant antibody, a neutralizing antibody, a humanized antibody, a human antibody, a Fab fragment, a F(ab′)₂ fragment, a Fd fragment, a Fv fragment, a scFv antibody, and the like.

A “IL-1α neutralizing antibody”, as used herein refers to an antibody whose binding to IL-1α results in the inhibition of the biological activity of IL-1α, as assessed by measuring one or more indicators of IL-1α, such as IL-1α-induced cellular activation or IL-1α binding to IL-1α receptors or IL-1α signaling or the response of a IL-1α reporter, etc. These indicators of biological activity can be assessed by standard in vitro or in vivo assays known in the art.

In certain embodiments, an IL-1α inhibitory agent useful the methods presented herein may be a commercially available IL-1α antibody. Any convenient commercially available IL-1α antibody may be employed, including but not limited to, e.g., MABp1 is a True Human monoclonal antibody (XBiotech, Austin, Tex.), anti-IL-1α antibody (Abcam, Cambridge, Mass.), and the like. In other instances, anti-IL-1α antibodies and IL-1α binding proteins useful in practicing the methods presented herein may include those antibodies and binding proteins described in U.S. Patent Pub. Nos. 20160024202, 20100040574A1, 20110071054A1, 20120045444A1, the disclosures of which are incorporated herein by reference.

In certain embodiments, an IL-1α inhibitory agent useful the methods presented herein may be an IL-1α soluble receptor (also sometimes referred to as a “IL-1 Trap”). Any convenient IL-1α soluble receptor may be employed, including but not limited to, e.g., Rilonacept (ARCALYST®, Tarrytown, N.Y.) and, e.g., those described in U.S. Pat. No. 8,114,394 the disclosure of which are incorporated herein by reference.

In certain embodiments, a IL-1α inhibitory agent useful the methods presented herein may be a small molecule IL-1α inhibitor. Such small molecule IL-1α inhibitors may be specific or non-specific IL-1α inhibitors.

Other useful IL-1α inhibitory agents include but are not limited to, e.g., Anakinra (Kineret®), SD118 (a.k.a. NSL-043, Sosei and NeuroSolutions), OMS-103HP (Omeros Corporation, Seattle Wash.), as well as those described in, e.g., U.S. Pat. No. 5,075,222 U.S. Patent Publication No: 20030049255A1 and PCT Publication No: WO1994006457A1, the disclosures of which are incorporated herein by reference.

The present methods include contacting a neuron and/or an oligodendrocyte with, e.g., by administering to a subject, a TNFα inhibitor. TNFα inhibitors will vary and may include agents that inactivate or otherwise prevent TNFα signaling, e.g., by directly binding TNFα and/or by preventing TNFα from binding its receptor (e.g., by binding Tumor necrosis factor receptor 1 (TNFR1), also known as tumor necrosis factor receptor superfamily member 1A (TNFRSF1A) and CD120a) in a manner that prevents TNFα binding and/or signaling), by preventing the expression of TNFα or by preventing (e.g., directly preventing) the release of TNFα from a TNFα-releasing cell (e.g., an A1 reactive astrocyte).

TNFα and TNFα signaling are well understood in the art and described in, e.g., Palladino et al. (2003) Nat Rev Drug Discov. 2(9):736-46; Barbara et al. (1996) Immunol Cell Biol. 74(5):434-43; Pickering et al. (1996) Immunol Cell Biol. 74(5):434-43; Pennica et al. (1984) Nature 312:724-729; Davis et al. (1987) Biochemistry 26:1322-1326; and Jones et al. (1989) Nature 338:225-228, the disclosures of which are incorporated herein by reference. Briefly, human TNFα is translated as a 26-kDa protein that lacks a classic signal peptide. Synthesized pro-TNFα expressed on the plasma membrane is cleaved through the action of matrix metalloproteinases to release a mature soluble 17-kDa TNFα. In both its cell-associated and secreted forms, trimerization is required for biological activity. Both the cell-associated 26-kDa and secreted 17-kDa forms are biologically active. Cell-associated TNF-α is processed to a secreted form by TNFα-converting enzyme (TACE; also referred to as ADAM-17).

The biological response to TNFα and TNFα signaling is mediated through receptors. Receptors for TNFα include transmembrane glycoproteins with multiple cysteine rich repeats in the extracellular N-terminal domains, e.g., type I receptors, e.g., Tumor Necrosis Factor Receptor 1 (TNFR1, a.k.a. p60, p55, CD120a), and type II receptors, e.g., Tumor Necrosis Factor Receptor 2 (TNFR2, a.k.a. p80, p75, CD120b). TNFα signaling through TNFR1 and TNFR2 may be either overlapping or distinct.

In some instances TNFα inhibitory agents (i.e., TNFα inhibitors) are agents that directly bind TNFα. TNFα inhibitory agents that directly bind to TNFα may inhibit various functions of TNFα including, but not limited to, binding of TNFα to a TNFα receptor, binding of TNFα to TNFα (e.g., trimerization), binding of TNFα processing agents thus inhibiting processing of TNFα (e.g., pro-TNFα processing, TACE TNFα processing, etc.), binding of TNFα cleaving agents thus inhibiting cleaving of TNFα (e.g., cleaving of TNFα at the cell membrane, metalloproteinases release of TNFα, etc.), and the like. In other instances, TNFα inhibitory agents are agents that directly bind TNFα may prevent TNFα from being expressed on the cell surface, e.g., by preventing newly translated TNFα from being transported to the cell membrane or by preventing modification of TNFα that allows TNFα to be expressed on the membrane.

In some instances, a TNFα inhibitory agent may interfere, directly or indirectly, with proteolytic processing of TNFα. For example, a TNFα inhibitory agent may interfere with proteolytic processing, including but not limited to, proteolytic processing of TNFα by metalloproteinases, proteolytic processing of TNFα by TACE, proteolytic processing of TNFα by signal peptide peptidase-like 2A (SPPL2A), proteolytic processing of TNFα by signal peptide peptidase-like 2B (SPPL2B), etc.

In some instances, a TNFα inhibitory agent may preferentially target either soluble or membrane tethered TNFα. For example, in some instances a TNFα inhibitory agent may preferentially bind soluble TNFα. In other instances a TNFα inhibitory agent may preferentially bind membrane tethered TNFα. In other instances a TNF-α inhibitory agent may preferentially prevent the production of soluble TNFα. In yet other instances, instances a TNFα inhibitory agent may preferentially prevent the production of membrane tethered TNFα. In certain embodiments, a TNFα inhibitory agent may preferentially prevent the function of soluble TNFα. In other embodiments, a TNFα inhibitory agent may preferentially prevent the function of membrane tethered TNFα.

In some instances, a TNFα inhibitory agent may interfere, directly or indirectly, with post-translational modification of TNFα and thus inhibit TNFα function. For example, a TNF-α inhibitory agent may interfere with or prevent TNFα phosphorylation, e.g., phosphorylation on serine residues, including but not limited to preventing phosphorylation of membrane bound TNFα. A TNFα inhibitory agent may interfere with or prevent TNFα dephosphorylation, e.g., dephosphorylation of serine residues, including but not limited to preventing dephosphorylation of membrane bound TNFα. In other instances, a TNFα inhibitory agent may interfere with other post-translational modifications of TNF-α or the reversal of other post-translational modifications of TNFα, including but not limited to, glycosylation, including but not limited to O-linked glycosylation, N-linked glycosylation, fatty acid acylation, defatty-acylation, and the like.

In some instances TNFα inhibitory agents are agents that directly bind a TNFα receptor and antagonize binding of TNFα to a TNFα receptor. Binding of a TNFα inhibitory agent to a TNFα receptor may block TNFα signaling through means other than preventing TNFα from binding its receptor including, e.g., preventing signal transduction.

In some instances a TNFα inhibitory agent may decrease the effective concentration of soluble TNFα. For example, in some instances a TNFα inhibitory agent may be a soluble form of or a solubilized portion of a TNFα receptor. Such agents that decrease the effective concentration of soluble TNFα bind or sequester soluble TNFα without activating TNFα signaling thus decreasing the amount of free soluble TNFα available to bind TNFα receptors capable of activating TNFα signaling.

In some instances a TNFα inhibitory agents may be an antibody or fragment thereof that directly binds to TNFα or a TNFα receptor, including but not limited to, e.g., an isolated antibody, a recombinant antibody, a neutralizing antibody, a humanized antibody, a human antibody, a Fab fragment, a F(ab′)₂ fragment, a Fd fragment, a Fv fragment, a scFv antibody, and the like.

A “TNFα neutralizing antibody”, as used herein refers to an antibody whose binding to TNFα results in the inhibition of the biological activity of TNFα, as assessed by measuring one or more indicators of TNFα, such as TNFα-induced cellular activation or TNFα binding to TNFα receptors or TNFα signaling or the response of a TNFα reporter, etc. These indicators of biological activity can be assessed by standard in vitro or in vivo assays known in the art.

In certain embodiments, a TNFα inhibitory agent useful the methods presented herein may be a commercially available TNFα antibody. Any convenient commercially available TNFα antibody may be employed, including but not limited to, e.g., Infliximab (REMICADE®, Janssen Biotech, Horsham, Pa.), a chimeric antibody having murine anti-TNFα variable domains and human IgG₁ constant domains; Adalimumab (HUMIRA®, Abbott Laboratories, Abbott Park, Ill.), a recombinant, fully human anti-TNFα antibody that binds specifically to TNFα and blocks its interaction with TNFα receptors; CDP-571 (Humicade™), D2E7, CDP-870, and the like. In other instances, anti-TNFα antibodies and TNFα binding proteins useful in practicing the methods presented herein may include those antibodies and binding proteins described in U.S. Pat. Nos. 8,722,860, 7,981,414 and 6,090,382, U.S. Patent Pub. Nos. 2006/0024308 and 2004/0033228, and PCT Pub. Nos. WO02002080892A1, WO2006014477A1 and WO2013063114A1, the disclosures of which are incorporated herein by reference.

In certain embodiments, a TNFα inhibitory agent useful the methods presented herein may be a commercially available TNFα soluble receptor. Any convenient commercially available TNFα soluble receptor may be employed, including but not limited to, e.g., Etanercept (ENBREL®, Amgen Inc., Thousand Oaks, Calif.), a recombinant fusion protein comprising two p75 soluble TNF-receptor domains linked to the Fc portion of a human immunoglobulin; lenercept, pegylated TNF-receptor type I, TBP-1, and the like.

In other embodiments, a TNFα inhibitory agent useful the methods presented herein may be an engineered TNFα molecule. Such engineered TNFα molecules are known in the art and include, but are not limited to, engineered TNFα molecules which form trimers with native TNFα and prevent receptor binding (see, e.g., Steed et al. (2003) Science 301:1895-1898, WO 03/033720, and WO 01/64889, the disclosures of which are incorporated herein by reference).

Such TNFα inhibitory agents and methods for their use are discussed in, e.g., Weinberg & Buchholz. TNF-alpha Inhibitors: Milestones in Drug Therapy (2006) Springer Science & Business Media, the disclosure of which is incorporated herein by reference.

In certain embodiments, a TNFα inhibitory agent useful the methods presented herein may be a small molecule TNFα inhibitor. Such small molecule TNFα inhibitors may be specific or non-specific TNFα inhibitors and include but are not limited to, e.g., MMP inhibitors (i.e. matrix metalloproteinase inhibitors), TACE-inhibitors (i.e. TNF Alpha Converting Enzyme inhibitors), tetracyclines (e.g., doxycycline, lymecycline, oxitetracycline, tetracycline, minocycline and synthetic tetracycline derivatives, such as chemically modified tetracyclines), prinomastat (AG3340), batimastat, marimastat, BB-3644, KB-R7785, quinolones (e.g., norfloxacin, levofloxacin, enoxacin, sparfloxacin, temafioxacin, moxifloxacin, gatifloxacin, gemifloxacin, grepafloxacin, trovafloxacin, ofloxacin, ciprofloxacin, refloxacin, lomefloxacin, temafioxacin etc.), thalidomide, thalidomide derivatives, 3,6′-dithiothalidomide, selective cytokine inhibitors, CC-1088, CDC-501, CDC-801, Linomide (Roquininex®), lazaroids, non-glucocorticoid 21-aminosteroids (e.g., U-74389G (16-desmethyl tirilazad) and U-74500), cyclosporin, pentoxifyllin derivates, hydroxamic acid derivates, napthopyrans, phosphodiesterase I, II, III, IV, and V-inhibitors; CC-1088, Ro 20-1724, rolipram, amrinone, pimobendan, vesnarinone, SB 207499 (Ariflo®), melancortin agonists, HP-228, CT3, ITF-2357, PD-168787, CLX-1100, M-PGA, NCS-700, PMS-601, RDP-58, TNF-484A, PCM-4, CBP-1011, SR-31747, AGT-1, solimastat, CH-3697, NR58-3.14.3, RIP-3, Sch-23863, iloprost, prostacyclin, CDC-801 (Celgene), DPC-333 (Dupont), VX-745 (Vertex), AGIX-4207 (AtheroGenics), ITF-2357 (Italfarmaco), and the like.

In certain embodiments, the TNFα inhibitory agent is thalidomide or a derivative or analog thereof, including but not limited to, e.g., those described in Muller et al. (1996) J Med Chem 39(17):3238-40, the disclosure of which is incorporated herein by reference. In some instances, the TNFα inhibitory agent is an immune-modulatory drug or a derivative or analog thereof of which thalidomide is one non-limiting example. Other immune-modulatory drugs useful as a TNFα inhibitory agent according to the methods described herein include but are not limited to, e.g., lenalidomide and pomalidomide. The mechanisms through which thalidomide, and derivatives or analogs thereof, and immune-modulatory drugs, and derivatives or analogs thereof, inhibit TNFα and/or TNFα signaling are described in, e.g., Muller et al. (1996) J Med Chem 39(17):3238-40; Lopez-Girona et al. (2012) Leukemia 26(11): 2326-2335; Zhu et al. (2013) Leuk Lymphoma 54(4):683-7; Majumder et al. (2012) Curr Top Med Chem 12(13):1456-67; and Bodera & Stankiewicz (2011) Recent Pat Endocr Metab Immune Drug Discov 5(3):192-6, the disclosures of which are incorporated herein by reference.

Other useful TNFα inhibitory agents include but are not limited to, e.g., SSR150106 (Sanofi, Bridgewater, N.J.), TIMP-1, TIMP-2, adTIMP-1 (i.e., adenoviral delivered TIMP-1), adTIMP-2 (adenoviral delivered TIMP-2), prostaglandins; IL-10, which is known to block TNFα production via interferon-γ-activated macrophages (Oswald et al., 1992, Proc. Natl. Acad. Sci. USA 89:8676-8680), TNFR-IgG (Ashkenazi et al., 1991, Proc. Natl. Acad. Sci. USA 88:10535-10539); the murine product TBP-1 (Serono/Yeda), the vaccine CytoTAb (Protherics), the peptide RDP-58 (SangStat), antisense molecule 104838 (ISIS), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach, and those described in, e.g., U.S. Patent Publication No: 2009/0042875 A1 and PCT Publication No: WO 2002080892 A1, the disclosures of which are incorporated herein by reference.

The present methods include contacting a neuron and/or an oligodendrocyte with, e.g., by administering to a subject, a C1q inhibitor. C1q inhibitors will vary and may include agents that inactivate or otherwise prevent C1q signaling, e.g., by directly binding C1q and/or by preventing C1q from binding a C1q receptor (e.g., by binding a C1q receptor in a manner that prevents C1q binding and/or signaling) or by preventing the expression of C1q. Non-limiting examples of C1q receptors include e.g., Complement receptor type 1 (a.k.a. CR1, CD35, etc.), Complement component C1q receptor (a.k.a. CD93, C1qRₚ, etc.), C1qRo_{Ö2} (Ruiz et al. (1995) J. Biol. Chem. 270, 30627-30634), Complement component 1 Q subcomponent-binding protein (a.k.a. gC1qR, gC1qBP, etc.), Calreticulin (a.k.a. cC1qR, CRP55, etc.) and the like.

C1q and C1q signaling are well understood and described in, e.g., Kishore & Reid (2000) Immunopharmacology 49:159-170, Son et al. Immunol Res. (2015) 63(1-3):101-6 , Kouser et al. Front Immunol. (2015) 6:317, the disclosures of which are incorporated herein by reference. Briefly, C1q is a 400 kDa multi-subunit protein complex made of six C1qA chains, six C1qB chains, and six C1qC chains. C1q performs a diverse range of complement and non-complement functions. C1q associates with the proenzymes C1r and C1s to yield C1, the first component of the serum complement system. The collagen-like regions of C1q interact with the Ca²⁺-dependent Clr₂Cls₂ proenzyme complex, and efficient activation of C1 takes place on interaction of the globular heads of C1q with the Fc regions of IgG or IgM antibody present in immune complexes. C1q can also bind various ligands derived from self, non-self, and altered self and modulate the functions of non-immune cells including dendritic cells and microglia.

In some instances C1q inhibitory agents (i.e., C1q inhibitors) are agents that directly bind C1q. C1q inhibitory agents that directly bind to C1q may inhibit various functions of C1q including, but not limited to, binding of C1q to a C1q receptor, and the like. In other instances, C1q inhibitory agents are agents that directly bind C1q may prevent C1q from being functionally expressed, e.g., by preventing newly translated C1q from being transported to the cell membrane or by preventing assembly of C1q subunits into a functional multi-subunit complex that allows C1q to be functionally expressed. In some instances, C1q inhibitor agents may prevent the release of C1q from a C1q-releasing cell (e.g., an A1 reactive astrocyte). For example, in addition to other methods of preventing release, inhibiting the production or expression of C1q may in turn prevent its release.

In some instances C1q inhibitory agents are agents that directly bind a C1q receptor and antagonize binding of C1q to a C1q receptor. Binding of a C1q inhibitory agent to a C1q receptor may block C1q signaling through means other than preventing C1q from binding its receptor including, e.g., preventing signal transduction.

In some instances a C1q inhibitory agent may decrease the effective concentration of soluble C1q. For example, in some instances a C1q inhibitory agent may be a soluble form of or a solubilized portion of a C1q receptor. Such agents that decrease the effective concentration of soluble C1q bind or sequester soluble C1q, or a fragment thereof such as e.g., the globular domain of C1q or the collagen-like domain of C1q, without activating C1q signaling thus decreasing the amount of free soluble C1q available to bind C1q receptors capable of activating C1q signaling.

In some instances a C1q inhibitory agent may be an antibody or fragment thereof that directly binds to C1q or a C1q receptor, including but not limited to, e.g., an isolated antibody, a recombinant antibody, a neutralizing antibody, a humanized antibody, a human antibody, a Fab fragment, a F(ab′)₂ fragment, a Fd fragment, a Fv fragment, a scFv antibody, and the like.

A “C1q neutralizing antibody”, as used herein refers to an antibody whose binding to C1q results in the inhibition of the biological activity of C1q, as assessed by measuring one or more indicators of C1q, such as C1q-induced cellular activation or C1q binding to C1q receptors or C1q signaling or the response of a C1q reporter, etc. These indicators of biological activity can be assessed by standard in vitro or in vivo assays known in the art.

In certain embodiments, a C1q inhibitory agent useful the methods presented herein may be a commercially available C1q antibody. Any convenient commercially available C1q antibody may be employed, including but not limited to, e.g., monoclonal antibodies and polyclonal antiserum to human C1q protein available from Quidel Corporation (San Diego, Calif.), anti-C1q antibodies and anti-C1q Fab (e.g., available from Creative Labs, Shirley, N.Y.), and the like. In other instances, anti-IL-1α antibodies and IL-1α binding proteins useful in practicing the methods presented herein may include those antibodies and binding proteins described in e.g., Phuan et al. Acta Neuropathol. (2013) 125(6): 829-840; U.S. Patent Pub. Nos. 20160159890, 20160053023 and 20050019326, the disclosures of which are incorporated herein by reference.

In certain embodiments, a C1q inhibitory agent useful the methods presented herein may be an C1q soluble receptor. Any convenient C1q soluble receptor may be employed, including but not limited to, e.g., those described in Klickstein et al. Immunity. 1997 7(3):345-55 and Peerschke et al. Blood Coagul Fibrinolysis. 1998 9(1):29-37, the disclosures of which are incorporated herein by reference.

In certain embodiments, a C1q inhibitory agent useful the methods presented herein may be a small molecule C1q inhibitor. Such small molecule C1q inhibitors may be specific or non-specific C1q inhibitors and include peptide or non-peptide small molecules. Small molecule C1q inhibitors include but are not limited to e.g., bisphenol disulfates, steroids and triterpenoids. Peptide C1q inhibitors include but are not limited to e.g., cyclic peptide 2J ([CEGPFGPRHDLTFC]W, SEQ ID NO:1), human beta-defensin 2 (LPGVFGGIGDPVTCL, SEQ ID NO:2). Useful C1q inhibitors include but are not limited to e.g., those described in Qu et al. Mol Immunol. 2009 47(2-3): 185-195, the disclosure of which is incorporated herein by reference in its entirety.

In some instances, the anti-C1q agent employed may be an anti-C1q agent as described in U.S. Pat. Nos. 9,382,314; 9,382,313; 9,149,444, the disclosures of which are incorporated by reference herein in their entirety.

Other useful C1q inhibitory agents include but are not limited to, e.g., decorin (e.g., as described in U.S. Pat. No. 5,650,389), anti-C1q aptamers (e.g., as described in U.S. Patent Pub. No. 20090269356), C1q antagonists described in U.S. Patent Pub. No. 20090232794, C1q inhibitory compounds described in U.S. Patent Pub. No. 20070243187, the disclosures of which are incorporated herein by reference.

In some instances inhibitors of the subject targets, i.e., IL-1α, TNFα and C1q, may be one or more interfering nucleic acid. For example, an inhibitory agent may be an IL-1α, TNFα or C1q interfering nucleic acid or a nucleic acid that interferes with the function or production of IL-1α, TNFα or C1q. Interfering nucleic acids useful in practicing the methods disclosed herein include, but are not limited to, e.g., dsRNA, siRNA, shRNA, ddRNAi, and the like.

Interfering nucleic acid useful in certain embodiments for practicing methods described herein may be generated using in vitro, in vivo, or synthetic production methods. For example, in vitro production may be achieved by cloning an interfering nucleic acid construct in to an appropriate vector, e.g., a plasmid or phage DNA, used to generate the interfering nucleic acid and the interfering nucleic acid is generated through the use of an in vitro transcription reaction. Any convenient method for vitro transcription may find use in generating an interfering nucleic acid of the subject disclosure including, but not limited to, an in vitro transcription kit or a dsRNA synthesis kit. Non-limiting examples of commercially available in vitro transcription kits and dsRNA synthesis kits include MEGAscript® RNAi Kits (Life Technologies, Grand Island, N.Y.), Replicator RNAi Kits (Thermo Scientific®, a division of Fisher Scientific®, Pittsburgh, Pa.), T7 RiboMAX™ (Promega Corporation, Madison, Wis.), MAXIscript® (Life Technologies, Grand Island, N.Y.), T7 High Yield RNA Synthesis Kit (New England Biolabs, Ipswich, Mass.), SP6/T7 Transcription Kit (Roche Applied Science, Indianapolis, Ind.), and the like.

In vivo production of an interfering nucleic acid for use in certain embodiments of the methods described herein include but are not limited to methods of transforming a interfering nucleic acid producing construct (e.g., an expression vector comprising a nucleotide sequence encoding an interfering nucleic acid) into an organism, e.g., a phage, a virus, a prokaryote, a eukaryote, a bacterium, a yeast, a cell of a cell culture system, a cell of a mammalian cell culture system, a plant, a cell of a plant cell culture system, and the like, for the purpose of generating an interfering nucleic acid in vivo. Methods for production of an interfering nucleic acid in vivo, e.g., by introducing a dsRNA construct or a shRNA construct into a living cell by transformation of dsRNA constructs, are well known in the art, see, e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; U.S. Pat. Nos. 6,506,559; and 7,282,564, the disclosures of which are incorporated herein by reference. Non-limiting examples of commercially available in systems and materials for shRNA production include Knockout™ Inducible RNAi Systems (Clontech, Mountain View, Calif.), psiRNA™ Vectors (InvivoGen, San Diego, Calif.), MISSIONS siRNA and shRNA systems (Sigma-Aldrich Co., St. Louis, Mo.), and the like.

In certain embodiments, an interfering nucleic acid may be introduced into an organism through the use of a virus vector, e.g., a lentivirus vector. Such methods for introducing interfering nucleic acids using virus vectors and lentivirus vectors are well-known in the art. For example, in some cases, an expression vector comprising a nucleotide sequence encoding an interfering nucleic acid is a virus-based vector, e.g., a lentivirus vector, an adenovirus vector, an adeno-associated virus vector, etc. In some cases, an expression vector comprising a nucleotide sequence encoding an interfering nucleic acid includes a promoter operably linked to the nucleotide sequence encoding the interfering nucleic acid. Suitable promoters include constitutive promoters and inducible promoters.

Synthetic production of an interfering nucleic acid for use in certain embodiments of the methods described herein include but are not limited to methods of synthetic siRNA production.

In some embodiments, siRNA is produced by methods not requiring the production of dsRNA, e.g., chemical synthesis or de novo synthesis or direct synthesis. Chemically synthesized siRNA may be synthesized on a custom basis or may be synthesized on a non-custom or stock or pre-designed basis. Custom designed siRNA are routinely available from various manufactures (e.g., Ambion®, a division of Life Technologies®, Grand Island, N.Y.; Thermo Scientific, a division of Fisher Scientific®, Pittsburgh, Pa.; Sigma-Aldrich®, St. Louis, Mo.; Qiagen®, Hilden, Germany; etc.).

Methods for design and production of siRNAs to a target are known in the art, and their application to inhibition for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference. In vivo and vitro methods for RNAi targeted at TNF-α, for example, are described in, e.g., Salako et al. (2011) Mol Ther 19(3):490-9, Wilson et al. (2010) Nat Mater 9(11):923-8, Jakobsen et al. (2009) Molecular Therapy 17(10):1743-1753, Qin et al. (2011) Artificial Organs 35(7):706-714, the disclosures of which are incorporated herein by reference.

The subject agents and/or compositions may be administered locally or systemically. For example, in some instances, an agent or composition may be administered locally to a site where e.g., prevention or neuronal and/or oligodendrocyte death is desired, prevention of A1 astrocyte formation is desired, etc. Such sites where local delivery may be desired include but are not limited to e.g., a site of injury (e.g., a site of SCI), a site of degeneration (e.g., the eye in the case of eye-related neurodegeneration, the brain in the case of brain-related neurodegeneration, etc.) a site of neuroinflammation, and the like. In some embodiments, local delivery may be employed in the case of SCI. In some embodiments, local delivery may be employed in the case of stroke. In some embodiments, local delivery may be employed in the case of glaucoma. In some embodiments, local delivery may be employed in the case of neurodegenerative disease.

In some instances, local administration may be away from a primary site of injury, a primary site of degeneration or a primary site of neuroinflammation. For example, following an injury occurring at a primary site, e.g., a site in the spinal cord, local administration may be employed away from the primary site of injury, including e.g., where the administration is the brain or a portion thereof when the primary site of injury is in the spinal cord. In some instances, where the primary site of injury is the brain or a portion thereof, local administration may be employed at a site other than the brain or away from the brain, e.g., the spinal cord. As noted above, in some instances administration may be systemic, including e.g., where the condition affects the body systemically or portions of the subject throughout the body.

### Promoting Neuronal or Oligodendrocyte Death

As summarized above, the present methods include promoting neuronal or oligodendrocyte death. For example, in some instances, methods may be performed to locally prevent neuronal or oligodendrocyte growth and proliferation, e.g., by locally administering one or more agents that promote oligodendrocyte death to a location of a subject where neuronal and/or oligodendrocyte death is desired. Approaches to inducing oligodendrocyte death will vary, e.g., depending on the subject's condition, or the particular agents employed. Induction of neuronal and/or oligodendrocyte death is not limited to in vivo contexts, e.g., locally inducing death in a subject, but may also be employed to induce death in an in vitro context, e.g., to screen for one or more agents that inhibits neuronal and/or oligodendrocyte death, as described below.

Neuronal and/or oligodendrocyte death may be induced by promoting the local formation of A1 reactive astrocytes, e.g., in a region of a subject where neuronal and/or oligodendrocyte killing is desired. In some instances, one or more A1 reactive astrocyte inducing agents may be locally administered to a subject to promote local neuronal and/or oligodendrocyte killing. Useful A1 reactive astrocyte inducing agents may include but are not limited to e.g., IL-1α or agonists thereof, TNFα or agonists thereof, C1q or agonists thereof and combinations thereof. Such A1 reactive astrocyte inducing agents may be administered directly or recombinantly expressed, e.g., through the introduction of an expression construct that encodes IL-1α, TNFα, C1q or a combination thereof.

Neuronal and/or oligodendrocyte death may be induced by locally administering a neurotoxic composition. Such neurotoxic compositions will vary and may include but are not limited to e.g., an A1 reactive astrocyte conditioned medium. As described herein, A1 reactive astrocytes have been found to secrete a neurotoxin that promotes the death of CNS neurons and oligodendrocytes. Accordingly, methods of the present disclosure may include contacting CNS neurons and/or oligodendrocytes with the A1 reactive astrocyte produced neurotoxin, in various forms including as part of a condition medium, as part of a neurotoxin enriched composition, as a purified neurotoxin, etc., to promote neuron and/or oligodendrocyte death. Neurotoxins of the present disclosure, e.g., for use in one or more of the present methods, may be a protein neurotoxin or a proteinaceous component of a neurotoxin including where e.g., such protein neurotoxins or proteinaceous component(s) thereof are protease and heat sensitive. In some instances, a neurotoxin of interest, i.e., an A1 reactive astrocyte derived neurotoxin, is 30 kD in size or greater. Neurotoxins of the present disclosure may be administered directly or may be prepared as a pharmaceutical composition, e.g., as described in more detail below.

Methods of promoting neuronal and/or oligodendrocyte death may find use in a variety of settings including but not limited to e.g., instances where a subject may have detrimental neuronal activity, including e.g., where the presence of the detrimental neuronal activity is the cause of a subject's medical condition or a symptom thereof. Among conditions having detrimental neuronal activity which may benefit from neuronal and/or oligodendrocyte killing of the present methods are conditions involving detrimental neuronal activity causing pain (e.g., chronic pain), epilepsy, anxiety, addiction, and the like.

Chronic pain has many origins, including e.g., neuropathic pain (i.e., pain having neural origins caused by damage or disease affecting the somatosensory nervous system). Neuropathic pain can be if peripheral origin or originate in the CNS, including the brain and spinal cord. Pain derived from the CNS may include pain that is not a direct result of injury (e.g., Fibromyalgia) and pain that is a direct result of injury (e.g., chronic pain after traumatic brain injury; see e.g., Nampiaparampil JAMA. (2008) 300(6):711-9).

Epilepsy is a recurrent, paroxysmal disorder of cerebral function characterized by sudden, brief attacks of altered consciousness, motor activity, sensory phenomena, or inappropriate behavior caused by excessive discharge of cerebral neurons. Manifestations depend on the type of seizure, which may be classified as partial or generalized. In partial seizures, the excess neuronal discharge is contained within one region of the cerebral cortex. In generalized seizures, the discharge bilaterally and diffusely involves the entire cortex. Sometimes a focal lesion of one part of a hemisphere activates the entire cerebrum bilaterally so rapidly that it produces a generalized tonic-clonic seizure before a focal sign appears.

Most patients with epilepsy become neurologically normal between seizures, although overuse of anticonvulsants can dull alertness. Progressive mental deterioration is usually related to the neurologic disease that caused the seizures. Left temporal lobe epilepsy is associated with verbal memory abnormalities; right temporal lobe epilepsy sometimes causes visual spatial memory abnormalities. The outlook is best when no brain lesion is demonstrable.

Methods of the present disclosure include administering to a subject having a condition associated with detrimental neuronal activity a neurotoxic composition to induce killing of the neurons to which the detrimental neuronal activity is attributed. Promoting death of the neurons to which the detrimental neuronal activity is attributed may reduce the detrimental neuronal activity and/or treat one or more symptoms of the condition.

In some instances, one or more active agents of the present disclosure may be administered directly, e.g., surgically or by injection, to an area behind the blood brain barrier (BBB). In other instances the agent may be formulated to cross the BBB and thus making direct administration unnecessary. In certain circumstances, neither direct administration within the BBB nor functionalization of the agent to cross the BBB is necessary due to exposure of the underlying target neural tissue or permeabilization of the BBB. Exposure of the underlying target neural tissue and/or permeabilization of the BBB may result as a consequence of the specific condition or incidence from which a subject's condition is a result or may be purposefully caused as a means of administering the agent. In some instances exposure to trauma, e.g., traumatic brain injury or other CNS trauma (e.g., spinal cord injury, concussion, ischemia, etc.), may permeabilize the BBB allowing delivery across the BBB of an agent that is not functionalized to cross the BBB nor is directly delivered within the BBB. Conditions where the BBB of a subject is permissive to delivery of an agent including agents that have not been functionalized to cross the BBB may be determined by the ordinary skilled medical practitioner upon observation of the subject.

According to the methods as described herein an effective amount of an agent described herein may be administered to a subject, e.g., a subject having a condition as described herein in order to treat the subject for the condition. In some instances, an effective dose may be the human equivalent dose (HED) of a dose administered to a mouse, e.g., a twice daily does administered to a mouse. In some instances, the total amount contained in twice daily doses may be administered once daily.

Conversion of an animal dose to human equivalent doses (HED) may, in some instances, be performed using the conversion table and/or algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857; (available at www(dot)fda(dot)gov/cder/guidance/index(dot)htm, the disclosure of which is incorporated herein by reference).

TABLE 1
Conversion of Animal Doses to Human Equivalent Doses
Based on Body Surface Area

| | To Convert | | |
|---|---|---|---|
| | Animal Dose in | To Convert Animal Dose in mg/kg to | |
| | mg/kg to Dose | HED^{a} in mg/kg, Either: | |
| | in mg/m², | Divide | Multiply |
| Species | Multiply by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)0.33.
^{b}This kg value is provided for reference only since healthy children will rarely be volunteers for phase 1 trials.
^{c}For example, cynomolgus, rhesus, and stumptail.

The instant methods may include the co-administration of one or more active agents. The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Treatments described herein may be performed chronically (i.e., continuously) or non-chronically (i.e., non-continuously) and may include administration of one or more agents chronically (i.e., continuously) or non-chronically (i.e., non-continuously). Chronic administration of one or more agents according to the methods described herein may be employed in various instances, including e.g., where a subject has a chronic condition, including e.g., a chronic neurodegenerative condition (e.g., Alzheimer's disease, Huntington's disease, Parkinson's disease, amyotrophic lateral sclerosis, etc.), a chronic neuroinflammatory condition, etc. Administration of one or more agents for a chronic condition may include but is not limited to administration of the agent for multiple months, a year or more, multiple years, etc. Such chronic administration may be performed at any convenient and appropriate dosing schedule including but not limited to e.g., daily, twice daily, weekly, twice weekly, monthly, twice monthly, etc. Non-chronic administration of one or more agents may include but is not limited to e.g., administration for a month or less, including e.g., a period of weeks, a week, a period of days, a limited number of doses (e.g., less than 10 doses, e.g., 9 doses or less, 8 doses or less, 7 doses or less, etc., including a single dose).

The route of administration may be selected according to a variety of factors including, but not necessarily limited to, the condition to be treated, the formulation and/or device used, the patient to be treated, and the like. Routes of administration useful in the disclosed methods include but are not limited to oral and parenteral routes, such as intravenous (iv), intraperitoneal (ip), rectal, topical, ophthalmic, nasal, and transdermal. Pharmaceutical compositions formulated for particular routes of delivery are described in more detail elsewhere herein.

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject being treated.

Therapeutically effective doses of a subject compound or pharmaceutical composition can be determined by one of skill in the art, with a goal of achieving local (e.g., tissue) concentrations that are at least as high as the IC50 of an applicable compound disclosed herein.

The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

### Methods of Screening

As summarized above, methods of the present disclosure include identifying a neurotoxin and/or an inhibitor thereof, e.g., where such identifying is facilitated by one or more screening protocols. Screening of the present disclosure may include the production and/or use of A1 reactive astrocytes where such astrocytes may be in vivo or in vitro derived. For example, in some instances, the present methods may include inducing the formation of A1 reactive astrocytes in an animal and collecting the generated A1 reactive astrocytes to perform a screen. Methods of inducing A1 reactive astrocyte formation in vivo will vary and may include e.g., inducing an injury or other condition in the animal to promote the formation of A1 reactive astrocytes. In some instances, A1 reactive astrocytes may be induced in vivo without injury or other condition (e.g., neurodegenerative condition, neuroinflammatory condition, etc.) that promotes their formation, as A1 reactive astrocytes may be generate in vivo by administering IL-1α and TNFα or IL-1α, TNFα and C1q, or agonists thereof. In vivo generated A1 reactive astrocytes may be subsequently cultured, e.g., by preparing a primary culture of A1 reactive astrocytes in a suitable culture medium.

In some instances, A1 reactive astrocytes may be generated in vitro. The in vitro generation of A1 reactive astrocytes may or may not involve the primary culture of astrocytes or progenitors thereof. For example, in some instances, primary astrocytes or progenitors thereof may be collected from an animal and induced in culture to A1 reactive astrocyte fate. In some instances, an astrocyte cell line or a cell line of astrocyte progenitors (i.e., a non-primary culture) may be induced in culture to A1 reactive astrocyte fate. Whether derived from a primary or non-primary culture, in vitro induction of A1 reactive astrocyte fate may involve contacting the cells with a suitable culture medium containing IL-1α and TNFα or IL-1α, TNFα and C1q, or agonists thereof.

In some instances, A1 reactive astrocytes may be derived from pluripotent progenitor cells. Useful pluripotent progenitor cells include but are not limited to e.g., non-autologous pluripotent progenitor cells or autologous pluripotent progenitor cells including but not limited to, e.g., newly derived embryonic stem cells (ESC) (including, e.g., those derived under xeno-free conditions as described in, e.g., Lei et al. (2007) Cell Research, 17:682-688) and newly derived induced pluripotent stem cells (iPS). General methods of inducing pluripotency to derive pluripotent progenitor cells are described in, e.g., Rodolfa K T, (2008) Inducing pluripotency, StemBook, ed. The Stem Cell Research Community, doi/10.3824/stembook.1.22.1 and Selvaraj et al. (2010) Trends Biotechnol, 28(4)214-23, the disclosures of which are incorporated herein by reference. In some instances, pluripotent progenitor cells, e.g., iPS cells, useful in the methods described herein are derived by reprogramming and are genetically unmodified, including e.g., those derived by integration-free reprogramming methods, including but not limited to those described in Goh et al. (2013) PLoS ONE 8(11): e81622; Awe et al (2013) Stem Cell Research & Therapy, 4:87; Varga (2014) Exp Cell Res, 322(2)335-44; Jia et al. (2010) Nat Methods, 7(3):197-9; Fusaki et al. (2009) Proc Jpn Acad Ser B Phys Biol Sci. 85(8):348-62; Shao & Wu, (2010) Expert Opin Biol Ther. 10(2):231-42; the disclosures of which are incorporated herein by reference.

In some instances, A1 reactive astrocytes useful in the methods of screening described herein may be derived from a subject such that the screen may be performed in a patient-specific way, e.g., in accordance with approaches related to personalized medicine. For example, A1 reactive astrocytes or precursors thereof (e.g., resting astrocytes) may be derived from cells obtained from a subject (e.g., cells obtained from the subject and used to generate iPS cells) and the patient-specific A1 reactive astrocytes or precursors thereof may be screened, e.g., to identify an agent effective in preventing the formation of A1 reactive astrocytes or preventing A1 reactive astrocyte mediated cell (e.g., neuron or oligodendrocyte) death in a patient-specific way. In some instances, the cells assayed in a patient-specific way may be obtained from a patient having a condition associated with A1 reactive astrocytes, including but not limited to e.g., those conditions described herein (e.g., a neurodegenerative condition (e.g., Alzheimer's disease, Huntington's disease, Parkinson's disease, amyotrophic lateral sclerosis, Multiple Sclerosis, MND, SCA, SMA, etc.), a neuroinflammatory condition (e.g., ADEM, ON, Transverse Myelitis, NMO, etc.), eye-related neurodegenerative disease (e.g., glaucoma, diabetic retinopathy, age-related macular degeneration (AMD), etc.), and the like).

Accordingly, in some instances, astrocytes may be generated from pluripotent progenitor cells and such cells may be induced to A1 astrocyte fate including e.g., by contacting the generated astrocytes with IL-1α and TNFα or IL-1α, TNFα and C1q.

The obtainment of A1 reactive astrocyte fate may be confirmed by various different methods including but not limited to e.g., introducing a sample of the medium in which the cells are cultured and testing the sample for neurotoxic characteristics, directly analyzing the cells of a portion thereof (e.g., by assessing the gene expression of one or more A1 reactive astrocyte markers), and the like. Conventional methods of cell culture and conventional culture conditions for maintaining neuronal cell types, including neurons, oligodendrocyte, astrocytes, etc., whether from established cell lines or primary culture may be employed and/or modified for use in the herein described methods.

The culture medium within which A1 reactive astrocytes are cultured may be collected, utilized and/or analyzed for various purposes. For example, in some instances, A1 reactive astrocyte conditioned medium may be employed to identify and/or isolate one or more A1 reactive astrocyte secreted neurotoxins. Various methods may be employed to identify and/or isolate such neurotoxins. For example, A1 reactive astrocyte conditioned medium, or components thereof, may be fractionated and the fractions assayed for neurotoxic function (e.g., as compared to appropriate control(s)). Fractions identified as having neurotoxic function may be considered to be “enriched” for the neurotoxic compound. Enriched factions may be utilized directly, e.g., in one or more methods of promoting neuronal or oligodendrocyte death or may be further analyzed or used in methods of purifying the neurotoxin.

A1 astrocyte conditioned medium, whether or not fractionated, may be subjected to one or more rounds of component characterization. Characterization of the components of A1 astrocyte conditioned medium may be performed by any convenient and appropriate method. Non-limiting examples of methods that may be employed in characterizing the components of A1 astrocyte conditioned medium include high performance liquid chromatography (HPLC), mass spectrometry (MS), liquid chromatography MS (LC/MS), and the like. Such approaches may or may include a proteomics component, e.g., where the proteins produced in an A1 astrocyte conditioned medium are compared to the proteins produced in an appropriate control to identify the neurotoxin.

Comparative expression analyses may also find use in identifying a neurotoxin according to the herein described methods. For example, in some instances, highly pure, cell-type specific gene databases may be employed to identify receptors present on mature neurons and/or oligodendrocyte that are absent on cell types that are not susceptible to A1 reactive astrocyte derived neurotoxin. In embodiments of such an approach, an expression library representing the receptor repertoire of highly pure populations of mature neurons, mature oligodendrocyte or both that are sensitive to an A1 reactive astrocyte produced neurotoxin may be compared to one or more expression library representing the receptor repertoire of cells that are not sensitive to an A1 reactive astrocyte produced neurotoxin (e.g., immature oligodendrocytes, A1 astrocyte insensitive neurons, etc.). Receptors present in the neurotoxin sensitive library that are absent in the neurotoxin insensitive library may identify putative neurotoxin receptors. As such, in some instances, identifying a neurotoxin according to methods of the present disclosure may include an identification based on identifying a differentially expressed neurotoxin receptor.

Following identification of candidate neurotoxins (whether or not based on identifying a differentially expressed neurotoxin receptor), in some instances, the candidate neurotoxin may be purified and its ability to induce neuronal and/or oligodendrocyte cell killing may be assessed. The neurotoxin or one or more candidate neurotoxins may be purified from the A1 reactive astrocyte conditioned medium or, following identification, the identified neurotoxin may be recombinantly expressed to obtain highly purified neurotoxin.

Neurotoxin containing samples, including but not limited to e.g., samples containing purified neurotoxin or samples enriched for neurotoxin, samples of A1 reactive astrocyte conditioned medium, fractionated medium, etc., may be employed in methods of screening for one or more neurotoxin inhibitors. For example, according to embodiments of such methods, a culture of cells susceptible to the neurotoxin (e.g., mature neurons, mature oligodendrocyte, etc.) may be contacted with a neurotoxin containing sample in the presence of one or more candidate neurotoxin inhibitors. Following culture for a suitable period in the presence of both neurotoxin containing sample and one or more candidate neurotoxin inhibitors viability may be assessed. Any convenient method of assessing viability may be employed including but not limited to e.g., those employing a viability dye including but not limited to propidium iodide (PI), 7-amino-actinomycin D (7-AAD), and those available from commercial distributors such as Fixable Viability Dye eFluor 455UV/450/506/520/660/780 (Affymetrix eBioscience, San Diego, Calif.), LIVE/DEAD Fixable BlueNiolet/Aqua/Yellow stain (Life Technologies, Grand Island, N.Y.), Zombie Aqua/Green/NIR/RED/UV/Violet/Yellow (BioLegend, San Diego, Calif.) and the like.

Various methods of qualitatively or quantitatively assaying the viability of cells assayed as described above may be employed. For example, in some instances, microscopy, with or without a viability dye, may be employed and the relative amounts of viable and non-viable cells may be determined, e.g., by qualitative observations or quantitative methods. In some instances, automated methods of quantification may be employed including but not limited to e.g., automated microscopic image analysis, cytometric methods (e.g., flow cytometry, image cytometry, and the like). In some instances, automated screening methods may be employed to facilitate high throughput screening of candidate agents and/or high throughput validation of identified agents. Agents identified in the subject screens may be employed in any of the subject methods and/or compositions and/or kits described herein.

### Compositions

Also provided are compositions for use in the subject methods. The subject compositions include any combination of components for performing the subject methods. In some embodiments, a composition can include, but is not limited to and does not require, the following: IL-1α, TNFα, C1q, IL-1α inhibitor, TNFα inhibitor, C1q inhibitor, astrocyte conditioned medium (e.g., A1 reactive astrocyte conditioned medium), A1 reactive astrocyte derived neurotoxin, and/or any combination thereof. Accordingly, compositions of the present disclosure include neuroprotective compositions, neurotoxic compositions and the like. Such compositions may or may not be formulated as pharmaceutical compositions. Also provided are compositions useful in one or more methods of identifying a neurotoxin or one or more methods of identifying the inhibitor of a neurotoxin, e.g., as described herein. The present compositions may be configured for use as a single composition or as a collection of two or more compositions, e.g., as a kit of compositions as described below.

### Pharmaceutical Compositions

Aspects of the instant disclosure include pharmaceutical compositions for performing one or more of the methods described herein where such a pharmaceutical composition may include an IL-1α inhibitor and a TNFα inhibitor appropriately formulated for administration as described herein. In some instances, a pharmaceutical composition may include an IL-1α inhibitor, a TNFα inhibitor and a C1q inhibitor appropriately formulated for administration as described herein. The active agents of the subject pharmaceutical compositions (e.g., IL-1α inhibitor, TNFα inhibitor, C1q inhibitor) may be combined, i.e., as a composition of two or more active agents, or may be formulated individually into separate compositions. In some instances, pharmaceutical compositions individually formulated with each active agent may be provided in the form of a kit, as described below, for treating a subject with a combination treatment of two or more compositions each having one or more of the subject active agents (e.g., IL-1α inhibitor, TNFα inhibitor, C1q inhibitor).

A pharmaceutical composition comprising a subject compound may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilisate, tablet, pill, pellet, capsule, powder, syrup, elixir or any other dosage form suitable for administration.

A subject compound may be administered to the host using any convenient means capable of resulting in the desired reduction in disease condition or symptom. Thus, a subject compound can be incorporated into a variety of formulations for therapeutic administration. More particularly, a subject compound can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, etc.

Formulations for pharmaceutical compositions are well known in the art. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the subject compounds can be formulated for use in human or veterinary medicine. Particular formulations of a disclosed pharmaceutical composition may depend, for example, on the mode of administration and/or on the location of the affected area to be treated. In some embodiments, formulations include a pharmaceutically acceptable carrier in addition to at least one active ingredient, such as an IL-1α inhibitor, a TNFα inhibitor or a C1q inhibitor. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are conventional in the art. The nature of a pharmaceutical carrier will depend on the particular mode of administration being employed. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A subject compound can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Such preparations can be used for oral administration.

A subject compound can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. The preparation may also be emulsified or the active ingredient encapsulated in liposome vehicles. Formulations suitable for injection can be administered by an intravitreal, intraocular, intramuscular, subcutaneous, sublingual, or other route of administration, e.g., injection into the gum tissue or other oral tissue. Such formulations are also suitable for topical administration.

In some embodiments, a subject compound can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Furthermore, a subject compound can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

In some instances, the active agent is configured to cross the blood brain barrier. For example, the active agent may be conjugated to a moiety that confers upon the active agent the ability to cross the blood brain barrier. Such a configuration allows for the targeting of the active agent to tissues within the blood brain barrier. In some embodiments the subject moiety may be a peptide, e.g., vasoactive intestinal peptide analog (VIPa) or a cell-penetrating peptide. Suitable peptides that facilitate crossing of the blood brain barrier include, but are not limited to positively charged peptides with amphipathic characteristics, such as MAP, pAntp, Transportan, SBP, FBP, TAT₄₈₋₆₀, SynB1, SynB3 and the like.

In other embodiments, the subject moiety may be a polymer. Suitable polymers that facilitate crossing of the blood brain barrier include, but are not limited to, surfactants such as polysorbate (e.g., Tween® 20, 40, 60 and 80); poloxamers such as Pluronic® F 68; and the like. In some embodiments, an active agent is conjugated to a polysorbate such as, e.g., Tween® 80 (which is Polyoxyethylene-80-sorbitan monooleate), Tween® 40 (which is Polyoxyethylene sorbitan monopalmitate); Tween® 60 (which is Polyoxyethylene sorbitan monostearate); Tween® 20 (which is Polyoxyethylene-20-sorbitan monolaurate); polyoxyethylene 20 sorbitan monopalmitate; polyoxyethylene 20 sorbitan monostearate; polyoxyethylene 20 sorbitan monooleate; etc. Also suitable for use are water soluble polymers, including, e.g.: polyether, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly(vinyl pyrrolidinone) (“PVP”); poly(amino acids); dextran, and proteins such as albumin. Block co-polymers are suitable for use, e.g., a polyethylene oxide-polypropylene oxide-polyethylene-oxide (PEO-PPO-PEO) triblock co-polymer (e.g., Pluronic® F68); and the like; see, e.g., U.S. Pat. No. 6,923,986. Other methods for crossing the blood brain barrier are discussed in various publications, including, e.g., Chen & Liu (2012) Advanced Drug Delivery Reviews 64:640-665.

One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents is also an option. A BBB disrupting agent can be co-administered with the therapeutic compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir. Traversal of the BBB may also be achieved through transient disruption of the BBB using focused ultrasound (FUS) including but not limited to e.g., as described in Etame et al. Neurosurg Focus. 2012 32(1): E3; the disclosure of which is incorporated herein by reference in its entirety.

The term “unit dosage form,” as used herein, refers to physically discrete units suitable as unitary dosages for human and animal subjects, each unit containing a predetermined quantity of a subject compound calculated in an amount sufficient to produce the desired effect in association with a pharmaceutically acceptable diluent, carrier or vehicle. The specifications for a subject compound depend on the particular compound employed and the effect to be achieved, and the pharmacodynamics associated with each compound in the host.

The dosage form of a disclosed pharmaceutical composition will be determined by the mode of administration chosen. For example, in addition to injectable fluids, topical or oral dosage forms may be employed. Topical preparations may include eye drops, ointments, sprays and the like. In some instances, a topical preparation of a medicament useful in the methods described herein may include, e.g., an ointment preparation that includes one or more excipients including, e.g., mineral oil, paraffin, propylene carbonate, white petrolatum, white wax and the like, in addition to one or more additional active agents.

Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions comprising a subject compound may be formulated in unit dosage form suitable for individual administration of precise dosages. The amount of active ingredient administered will depend on the subject being treated, the severity of the affliction, and the manner of administration, and is known to those skilled in the art. Within these bounds, the formulation to be administered will contain a quantity of the extracts or compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual subject compound may be administered at the same time as another therapeutic compound or sequentially, in any order thereof.

### Kits

Also provided are kits for use in the subject methods. The subject kits include any combination of components and compositions for performing the subject methods. In some embodiments, a kit can include the following: a IL-1α inhibitor in a pharmaceutical composition and a TNFα inhibitor in a pharmaceutical composition with or without any additional agent as described herein, a pharmaceutical application device or delivery device; and any combination thereof. In some instances, a kit of the present disclosure may include a IL-1α inhibitor in a pharmaceutical composition, a TNFα inhibitor in a pharmaceutical composition and a C1q inhibitor in a pharmaceutical composition. In some instances, kits of the present disclosure may include a kit for generating A1 reactive astrocytes which may include e.g., IL-1α, TNFα and C1q with or without any additional agent as described herein e.g., for use in one or more of the methods described herein. In some instances, a kit of the present disclosure may include a neurotoxin secreted by A1 reactive astrocytes, e.g., in the form of a conditioned medium or in purified form with or without any additional agent as described herein.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

The following examples are offered by way of illustration and not by way of limitation.

### EXAMPLES

### Example 1: Activated Microglia Induce Neurotoxic Reactive Astrocytes Via II-1α, TNFα, and C1q

Unless specifically specified otherwise, the following animal models and methods are applicable to the results of this example described below.

Animals. Sprague Dawley rats were from Charles River. TNFα^{-/-} (B6.129S-Tnftm1Gkl/J) transgenic mice and wild type C57BL/6J mice were from Jackson Laboratories. C1q(a)^{-/-} (C57BL/6) were from previous studies in our lab³². II-1α^{-/-} mice were a gift from Dr Russell E. Vance, UC Berkeley. Tg(Aldh1I1-EGFP)OFC789Gsat/Mmucd mice were used to visualize astrocytes in in vivo phagocytic assays. All lines were maintained by breeding with C57BL/6 mice. Animals were randomly assigned numbers and evaluated thereafter blind (to both experimental condition and genotype).

Immunopanning and Cell Culture. Astrocytes were purified by immunopanning from postnatal day 5 rats or mice (see above) forebrains and cultured as previously described¹⁷. Briefly, cortices were enzymatically (papain) then mechanically dissociated to generate a single cell suspension that was incubated on successive negative immunopanning plates to remove microglia, endothelial cells, and oligodendrocyte lineage cells before positively selecting for astrocytes with an Itgb5-coated panning plate. Isolated astrocytes were cultured in a defined, serum-free base media containing 50% neurobasal, 50% DMEM, 100 units of penicillin, 100 μg/ml streptomycin, 1 mM sodium pyruvate, 292 μg/ml L-glutamine, 1×SATO and 5 μg/ml of N-acetyl cysteine. This media was supplemented with the astrocyte-required survival factor HBEGF (Peprotech, 100-47) at 5 ng/ml as previously descried¹⁷. A similar immunopanning protocol was used for other central nervous system cell types, with positive selection using THY1 (cortical neurons), 192 hybridoma clone (embryonic spinal motor neurons³³), CD31 (endothelial cells³⁴), O4 (oligodendrocyte lineage cells), PDGFRβ (pericytes³⁵), CD45 (microglia/macrophages). A1 reactive astrocyte were generated in vitro by growing purified astrocytes for 6 days and then treating for 24 h with II-1α (3 ng/ml, Sigma, 13901), TNFα (30 ng/ml, Cell Signaling Technology, 8902SF), and C1q (400 ng/ml, MyBioSource, MBS143105).

Microfluidic qRT-PCR. Total RNA was extracted from immunopanned cells using the RNeasy Plus kit (Qiagen) and cDNA synthesis performed using the SuperScript® VILO cDNA Synthesis Kit (Invitrogen, Grand Island, N.Y., USA) according to supplier protocols. We designed primers using NCBI primer blast software (www(dot)ncbi(dot)nlm(dot)nih(dot)gov/tools/primer-blast/) and selected primer pairs with least probability of amplifying nonspecific products as predicted by NCBI primer blast. All primers had 90-105% efficiency. We designed primer pairs to amplify products that span exon-exon junctions to avoid amplification of genomic DNA. We tested the specificity of the primer pairs by PCR with rat and mouse whole-brain cDNA (prepared fresh), and examined PCR products by agarose gel electrophoresis. For microfluidic qRT-PCR 1.25 μl of each cDNA sample was pre-amplified using 2.5 μl of 2× Taqman pre-amplification master mix (Applied Biosystems, Waltham, Mass., USA) and 1.25 μl of the primer pool (0.2 μmol each primer/μl, primer sequences for rat and mouse are provided in the below Tables 2-3). Pre-amplification was performed using a 10 min 95° C. denaturation step and 14 cycles of 15 s at 95° C. and 4 min at 60° C. Reaction products were diluted 5 times in TE Buffer (Teknova, Hollister, Calif., USA). Five microliters from a sample mix containing pre-amplified cDNA and amplification Master mix (20 mm Mgcl2, 10 mm dNTPs, FastStart Taq polymerase, DNA binding Dye loading reagent, 50×ROX, 20× Evagreen) was loaded into each sample inlet of a 96.96 Dynamic Array chip (Fluidigm Corporation, San Francisco, Calif., USA) and 5 μl from an assay mix containing DNA assay loading reagent, as well as forward and reverse primers (10 μmol/μl) was loaded into each detector inlet. The chip was then placed in the NanoFlex™ 4-IFC Controller (Fluidigm) for loading and mixing. After loading, the chip was processed in the BioMark™ Real-Time PCR System (Fluidigm) using a cycling program of 10 min at 95° C. followed by 40 cycles of 95° C. for 15 s and 60° C. for 30 s and 72° C. for 30 s. After completion of qPCR, a melting curve of amplified products was determined. Data were collected using BioMark™ Data Collection Software 2.1.1 build 20090519.0926 (Fluidigm) as the cycle of quantification (Cq), where the fluorescence signal of amplified DNA intersected with background noise. Fluidigm data were corrected for differences in input RNA using the geometric mean of three reference genes Aldh1I1, Gapdh, RpIp0. Data preprocessing and analysis was completed using Fluidigm Melting Curve Analysis Software 1.1.0 build 20100514.1234 (Fluidigm) and Real-time PCR Analysis Software 2.1.1 build 20090521.1135 (Fluidigm) to determine valid PCR reactions. Invalid reactions were removed from later analysis. Quantitative RT-PCR was conducted following the MIQE (minimum information for publication of quantitative real-time PCR experiments) guidelines³⁶. The array accommodated reactions for 96 samples and 96 genes in total. The pre-amplified cDNA samples from the stimulation experiments were measured together with no reverse transcriptase and no template controls on 96.96 Dynamic Array chips (Fluidigm). Cell-type specific transcripts were also detected for microglia, oligodendrocyte lineage cells, and neurons, with any astrocyte samples containing measurable levels of other cell types removed from further analysis. All primer sequences for rat and mouse are listed in the below Tables 2 and 3. Primer sequences for human as follows: hALDH1L1: FWD-AGGGGCTGTTTTTCTCTCGG (SEQ ID NO:3), REV-CATGGTAGCAGGAGGGTTGG (SEQ ID NO:4), hC3: FWD-AAAAGGGGCGCAACAAGTTC (SEQ ID NO:5), REV-GATGCCTTCCGGGTTCTCAA (SEQ ID NO:6).

Western blot. Protein samples (conditioned growth media) were collected at 4° C. in PBS buffer containing Complete Protease Inhibitor Cocktail (Roche) and concentrated with Amicon Ultra-15 centrifugal filter units, with a 30 kDa size exclusion (EMD Millipore). Total protein concentration of samples was determined via Bradford assay (Sigma) and equal amounts of total protein were loaded onto 12% Tris-HCl gels (Bio-Rad). Following electrophoresis (100V for 45 minutes), proteins were transferred to Immobilon-P membranes (EMD Millipore). Blots were probed overnight at 4° C. with 1:200 rabbit anti-GLYPICAN2 (abcam, ab129526), 1:200 rabbit anti-VERSICAN (abcam ab19345), 1:1000 rabbit anti-SYNDECAN1 (Invitrogen, 36-2900), 1:1000 rabbit anti-BREVICAN (MyBioSource, MBS710876), 1:1000 mouse anti-NEUROCAN (EMD Millipore, MAB5234), 1:200 mouse anti-NG2 (abcam, ab50009), 1:50 goat anti-mouse C1q (Santa Cruz Biotechnology, sc-365301). Blots were incubated with HRP-conjugated secondary antibodies at 1:5000 for 2 hours at room temperature and developed using ECL Prime Western Blotting Detection Reagent (GE Healthcare). Visualization and imaging of blots was performed with a FluorochemQ System (ProteinSimple).

Cytokine array screen. Conditioned media from immunopanned purified and cultured microglia grown in non-reactive, or LPS-induced reactive state was collected as above and 100 μg of total protein was incubated with a Rat Cytokine Antibody Array Kit (R&D Systems, ARY008) according to manufacturer protocols.

Immunohistochemistry. Animals are anaesthetized with a ketamine (100 mg/kg)/xylazine (20 mg/kg) cocktail, and perfused with PBS followed by 4% paraformaldehyde at approximately 70% cardiac output. Dissected brains were post-fixed overnight in 4% paraformaldehyde at 4° C., and cryoprotected in 30% sucrose. For retinal immunohistochemistry, whole eyeballs were dissected and placed in ice-cold 4% paraformaldehyde for 10 minutes, and then washed in dPBS before dissecting the retina away from the rest of the eyeball and post-fixing in 4% paraformaldehyde overnight a 4° C. For human control and MS tissue, samples were snap-frozen and briefly fixed in ice-cold methanol. Both brains and whole retinas were embedded in O.C.T. compound (Tissue-Tek) and 10 μm tissue sections were prepared with a Leica cryostat. The following antibodies were used: 1:5000 rabbit anti-GFAP (DAKO, Z0334), 1:500 rat anti-GFAP (Invitrogen, clone 2.2B10), rabbit anti-AQP4 (Sigma, HPA014784), 1:500 rabbit anti-RBPMS (PhosphoSolutions, 1830-RBPMS), 1:500 mouse anti-CD68 (AbD Serotec, clone 514H12), 1:500-1500 rabbit anti-hC3D (DAKO, A0063). Primary antibodies were visualized with appropriate secondary antibodies conjugated with Alexa fluorophore (Invitrogen).

Synapse formation assay. We purified retinal ganglion cells from postnatal day 5 rats by sequential immunopanning to greater than 99% purity and cultured them in serum-free medium as previously described³⁷. Control and A1 reactive astrocytes were plated on inserts and co-cultured with RGCs for 5-10 days. For quantification of structural synapses, RGCs were fixed and stained with antibodies against the presynaptic marker Bassoon and postsynaptic marker Homer. Synapse number and size were quantified by a custom-written MATLAB program¹⁵.

Survival/cell toxicity assay. Control or A1 reactive astrocytes (see above) were grown for 7 d in serum-free media supplemented with 5 ng/ml HBEGF¹⁷. Cells were then treated with II-1α, TNFα, and C1q or an equivalent volume of 1×dPBS and cells left for an additional 24 h. At this time, conditioned media was collected with cOmplete™, Mini, EDTA-free protease inhibitor cocktail (Sigma/Roche, 04693159001) and concentrated at 30 kDa with Amicon Ultra-15 Centrifugal Filter Units (Millipore, UFC903024) until approximately 30-50× concentrated. A Bradford assay was performed to determine total protein concentration, and 1-50 μg/ml total protein was added to purified cell cultures of neurons, oligodendrocytes, OPCs, endothelial cells, astrocytes, pericytes or microglia (plated at 1,000 cells/well in poly-d-lysine-coated (PDL) 96-well plates, grown for 5 days in serum-free base media) and viability assed using the LIVE/DEAD® Kits for mammalian cells (Thermo Fisher Scientific, L3224). Additional experiments were done on RGCs and oligodendrocytes using heat inactivated A1 ACM (20 minute treatment at 60° C.) or protease treatment of A1 ACM (1 U/ml plasmin from human plasma, Sigma-P1867), 2 h at room temperature. Protease treatment was halted with phenylmethylsulfonyl fluoride (Sigma, 78830) and aprotinin (Sigma, A4529)—final concentrations: 2 mM and 0.55 TIU/ml, respectively. Equivalent amounts of astrocyte base media proteins (BSA, transferrin, HBEGF etc.) were added back to protease-treated A1 ACM before treating cells). Viability was again assessed at 24 h as before. At least 6 independent experiments were conducted for each condition. For each experiment, 4 non-overlapping 20× fields per well were quantified in six wells.

Bacteria cultures and killing assays. Bacterial strains include Salmonella typhimurium (SL1344), Burkholderia thailandensis (E264), and Shigella flexneri (M90T). S. typhimurium was grown in LB broth (BD Biosciences, San Jose, Calif.). B. thailandensis and S. flexneri were grown in tryptic soy broth (TSB; BD Biosciences). All strains were grown in 2 mL broth overnight from a frozen stock with aeration at 37° C. Bacteria were subcultured 1:1000 into broth (S. typhimurim and S. flexneri into Mgm-MES media³ and B. thailandensis into TSB) and 50% supernatant from control astrocytes or A1 (bad) astrocytes at serial dilutions from 0-100 μg/mL. At 16 hours of growth the OD600 was recorded.

Retro-orbital nerve crushes. Postnatal day 7 and 14 Sprague Dawley rats or P21-28 mice were anaesthetized with 2.5% inhaled isoflurane in 2.0 L O₂/min. Without incision to the orbital rim, the supero-external orbital contents were blunt-dissected, the superior and lateral rectis muscles teased apart, and the left optic nerve exposed. The nerve was crushed for 3-5 seconds at approximately 2 mm distal to the lamina cribrosa. Animals also received and 2 μl intravitreal injection of neutralizing antibodies to II-1α (150 μg/μl, abcam, ab9614), TNFα (150 μg/μl, Cell Signaling Technology, #7321), and C1q (Quidel, A301), a rabbit IgG control (150 μg/μl, abcam, ab27472), or PBS at day 0 (the time of optic nerve crush, for 7 and 14 day experiments) or day 7 (for some 14 day experiments). Retinas were collected for qPCR analysis and immunofluoscent analysis at 7 and 14 days. After surgery, the eye fundus were checked to ensure retinal blood flow was inject. Retinas were collected for qPCR analysis and immunofluorescence at 7 days.

Oligodendrocyte proliferation and differentiation assays. Cultures of oligodendrocyte precursor cells (OPCs) were prepared by immunopanning and grown as outlined above. To measure proliferation, OPCs were grown for 24 hrs in OPC proliferation media 3′ and then changed into OPC media containing 10 μM EdU (ThermoFisher, C10339) and varying concentrations of A1 or resting ACM. After 5 days, the cells were fixed, permeabilized, and stained for EdU and DNA (Hoechst 33342) according to the protocol for the Click-Itm Edu Imaging Kit. To measure differentiations of OPCs into mature OLs 1 ug/ml A1 ACM was added to OPC cultures and they were imaged at 24 h intervals with phase time lapse microscope (IncuCyte Zoom® System). Images were analyzed and number of primary processes extending from the cell soma were counted. A cell was considered an OPC with 0-2 processes, a differentiating OL with 4-5 processes, and a mature OL with 5+ primary processes. Before differentiation into mature OLs, OPC migration was measured using the Template Matching and Slice Alignment and MTrackJ plugins for ImageJ.

Synaptosome/myelin purification and In vitro engulfment assay. Synaptosomes⁴⁰ and crude CNS myelin⁴¹ were purified as described previously, and conjugated with pHrodo™ Red, succinimidyl ester (Thermo Fisher Scientific, P36600) in 0.1 M sodium carbonate (pH 9.0) at room temperature with gentle agitation. After two hour incubation, unbounded pHrodo was washed-out by multiple rounds of centrifugation and pHrodo-conjugated synaptosomes/myelin were re-suspended with isotonic buffer containing 5% DMSO for subsequent freezing. Purified control and A1 reactive astrocytes from P6 rat pups (see above) were incubated with 5 μl pHrodo-conjugated synaptosomes for 24 h, or 800 μg/ml media pHrodo-conjugated myelin debris and imaged at 1 h intervals. Live astrocytes were imaged with epifluorescence time lapse microscope (IncuCyte Zoom® System) to reveal engulfed pHrodo-conjugated particles. For image processing analysis, we took 9 images/well using 20× objective lens from random areas of the 24 well plates and calculated the phagocytic index (PI) by measuring the area of engulfed synaptosomes/myelin (fluorescent signal) normalized to the area of astrocytes, using ImageJ. Relative engulfment ability was calculated by normalizing the PI of control (non-reactive) astrocytes by that of A1 reactive astrocytes³.

In vivo synapse engulfment assay. Tg(Aldh1I1-EGFP)OFC789Gsat/Mmucd transgenic mice were used to visualize astrocytes in all in vivo engulfment assays. Pups were anaesthetized with isoflurane and 5 mg/kg LPS was injected i.p. at postnatal day 3. Twenty hours later 1 μl of cholera toxin-β subunit (CTB) conjugated with Alexa594 (Invitrogen, 1 mg/ml in normal saline) was injected into the contralateral eye. After 24 h mice were sacrificed and half had the dorsal LGN dissected out for microfluidic qPCR analysis, while the remainder were perfused with PBS followed by 4% paraformaldehyde at 70% cardiac output and brains were dissected, post-fixed overnight for 4′C and transferred to 15% and 30% sucrose for 24 h each at 4° C. Brains were sectioned at 50 μm and floating coronal sections containing dLGN were mounted on slide glasses and used for analysis of the dLGN. For each dLGN, two fields (the tip and medial portions of dLGN that contain both contra- and ipsilateral projections) were imaged using Zeiss LSM510 inverted confocal microscopy to obtain 50-70 consecutive optical sections with 0.3 μm interval thickness. ImageJ was used to remove outliers (radius 2.0 pixels and threshold 20) from all channels and subtract background from CTB images (rolling bar radius 50 pixels). An image-processing algorithm (MATLAB, Mathworks) was used to localize CTB-labelled RGC projections engulfed by astrocytes by subtracting CTB-labelled projections outside of the Aldh1I1-eGFP positive cells. The phagocytic index was calculated by measuring the total volume of engulfed CTB-labelled RGC projections normalized to the total volume of astrocytes in a given z-stack. Relative engulfment ability was calculated by normalizing the phagocytic index of experimental groups to control group³.

Electrophysiology. Whole-cell patch-clamp recordings from cultured RGC neurons were performed at room temperature in an isotonic saline solution (in mM: NaCl 125, NaHCO₃25, KCl 2.5, NaH2PO4 1.25, glucose 25, MgCl2 1, CaCl2 2). Patch electrodes with resistances of 2.5-3.5 MO were pulled from thick-walled borosilicate glass capillaries and were filled with an internal solution containing (in mM) potassium gluconate 130, NaCl 4, EGTA 5, CaCl2 0.5, 10 HEPES, MgATP 4, Na2GTP 0.5 (pH 7.2 with KOH). Miniature excitatory postsynaptic currents (mEPSCs) were recorded in TTX (1 μM, Alomone) from a holding potential of −70 mV. Series resistance was monitored throughout the recording and was <20 MQ. Data were sampled at 50 kHz and filtered at 1 kHz using pClamp 9.2, and offline analysis of mEPSCs was performed using Clampfit 10.3 (Molecular Devices).

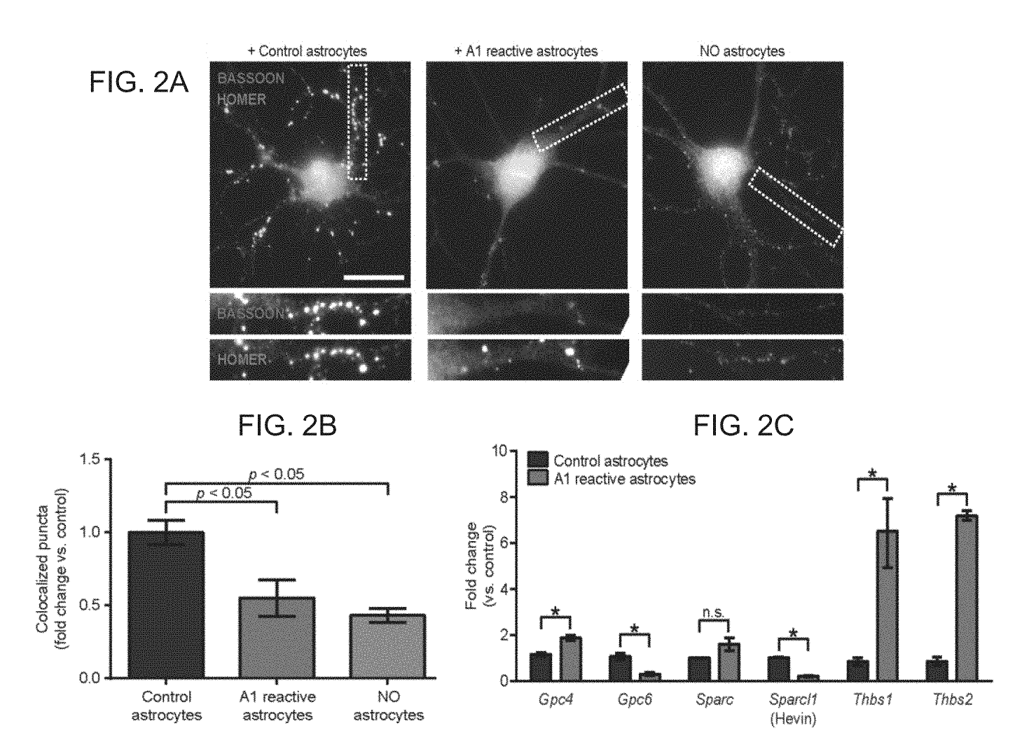

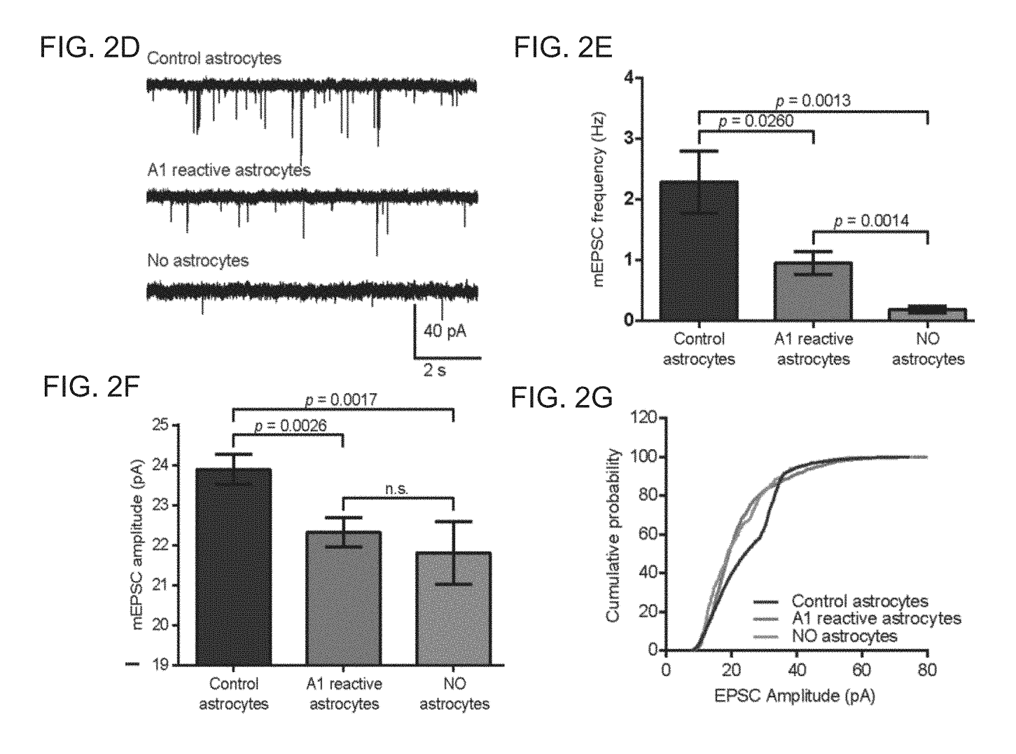

Statistical analysis and power calculations. All statistical analyses were done using GraphPad Prism 7.00 software. Most data were analyzed by one-way ANOVA followed by Dunnett's multiple post-hoc test for comparing more than three samples, and two-sample unpaired t-test for comparing two samples with 95% confidence. Two-sample Kolmogorov-Smimov test with 95% confidence was used for electrophysiology experiments in FIG. 2G. Power calculations were performed using G*Power Software V 3.1.9.2⁴². Group sizes were used to provide at least 80% calculable power with the following parameters: probability of Type I error (0.05), conservative effect size (0.25). Four to eight treatment groups with multiple measurements were obtained per replicate.

### Results

### Screen for Cellular and Molecular Inducers of the A1 Phenotype

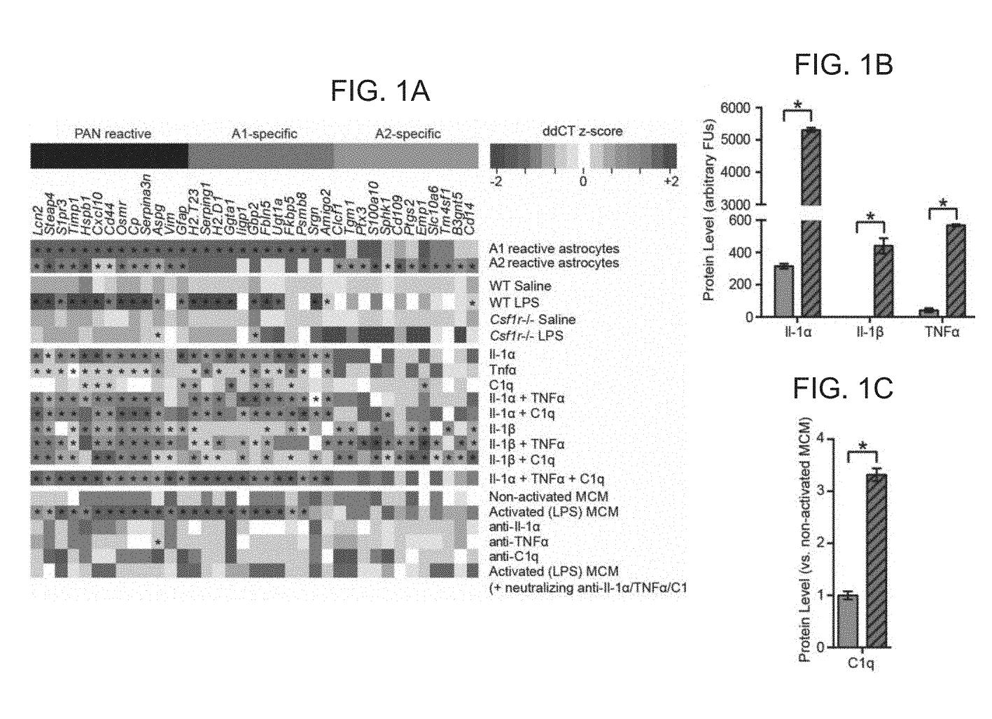

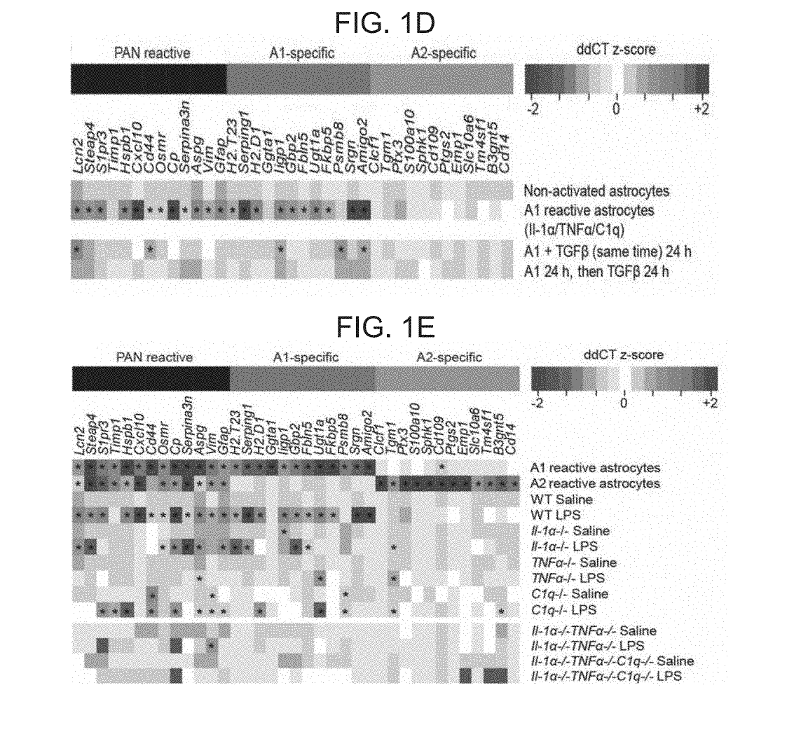

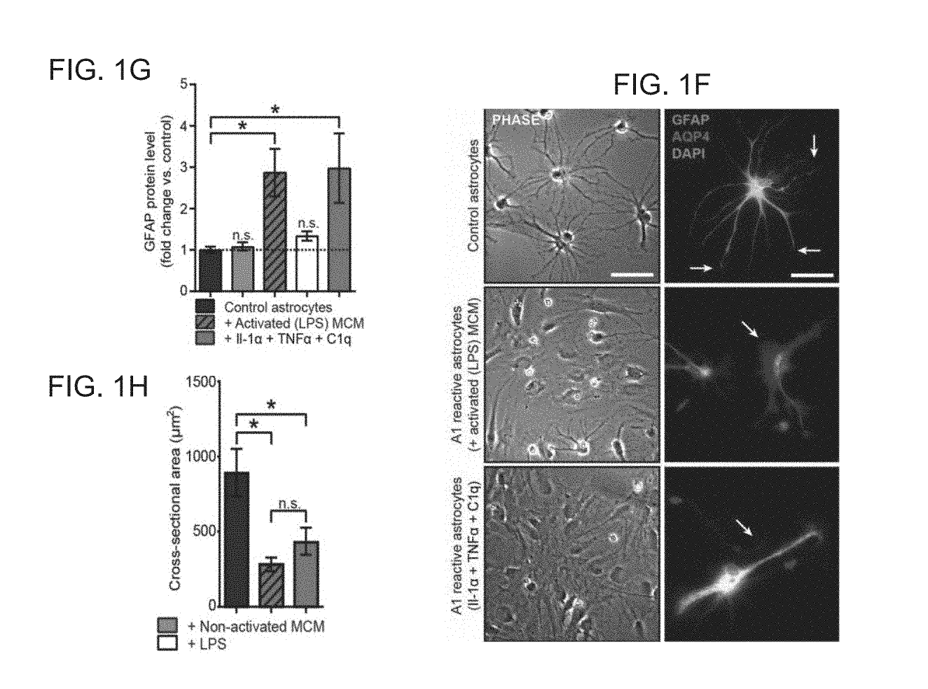

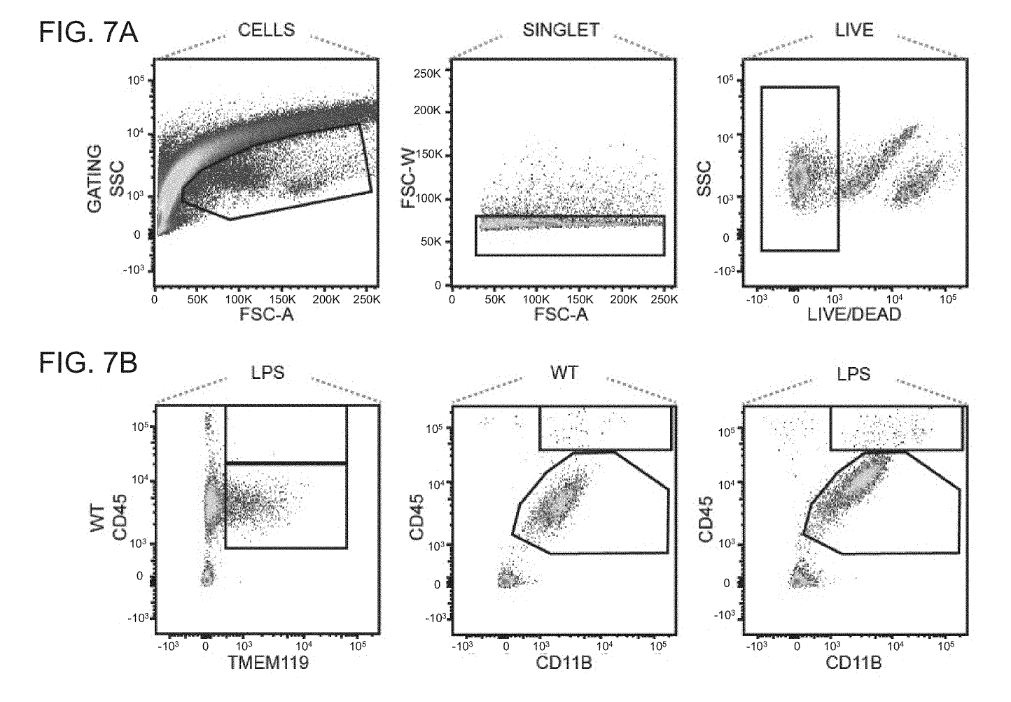

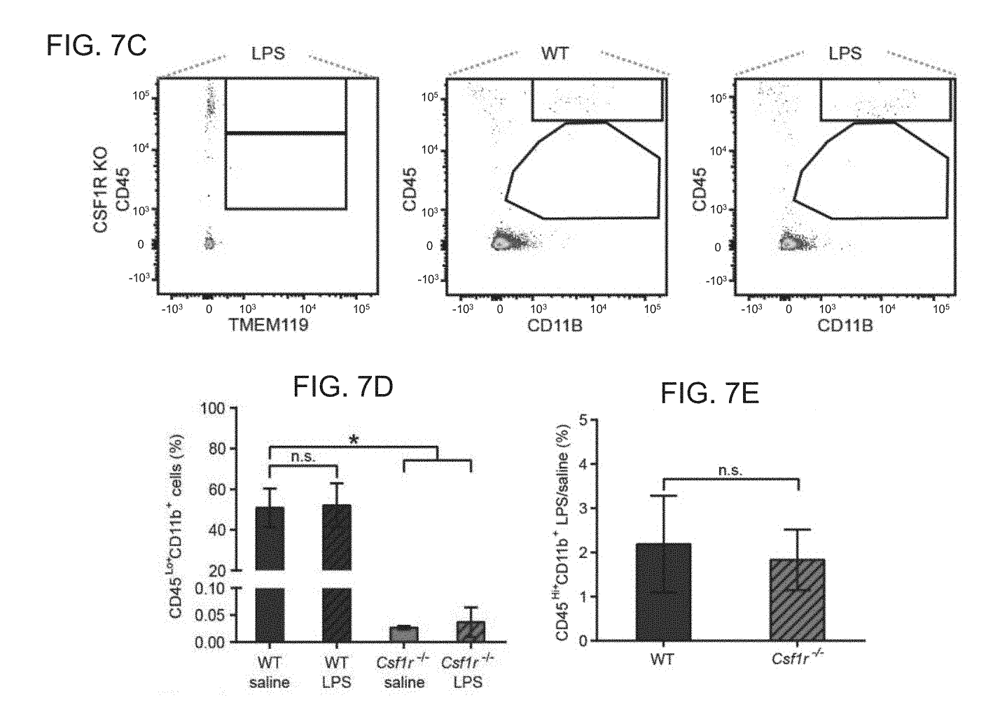

We first investigated whether microglia are the inducers of A1 reactive astrocytes because lipopolysaccharide (LPS) is a strong inducer of A1s and is an activator of TLR4 signaling, a receptor expressed specifically by microglial in our CNS cell type transcriptomes¹³⁻¹⁶. We took advantage of Csf1r^{-/-} knock-out mice that lack resident microglia¹² (see also FIG. 7A-7E), to ask whether systemic injection of LPS can induce A1s in mice that lack microglia. To assess whether astrocytes were reactive, we used a high-throughput microfluidic qPCR screen to determine gene expression changes in astrocytes purified by immunopanning from saline- and LPS-treated wild type control Csf1r^{-/-} mice. For this microfluidic assay we chose reactive astrocyte genes split into three broad classes: PAN reactive transcripts upregulated regardless of mode of induction; A1-specific transcripts only induced following neuroinflammation; and A2-specific transcripts only induced following ischemic injury⁵ (see also FIG. 1A-1H). As expected, wild type littermate controls had a normal response to LPS injection, with robust induction of an A1 reactive astrocyte response as shown by upregulation of PAN- and A1-specific transcripts (FIG. 1A), however astrocytes from Csf1r^{-/-} mice failed to activate to an A1 phenotype. These findings show that reactive microglia are required to induce A1 reactive astrocytes in vivo.

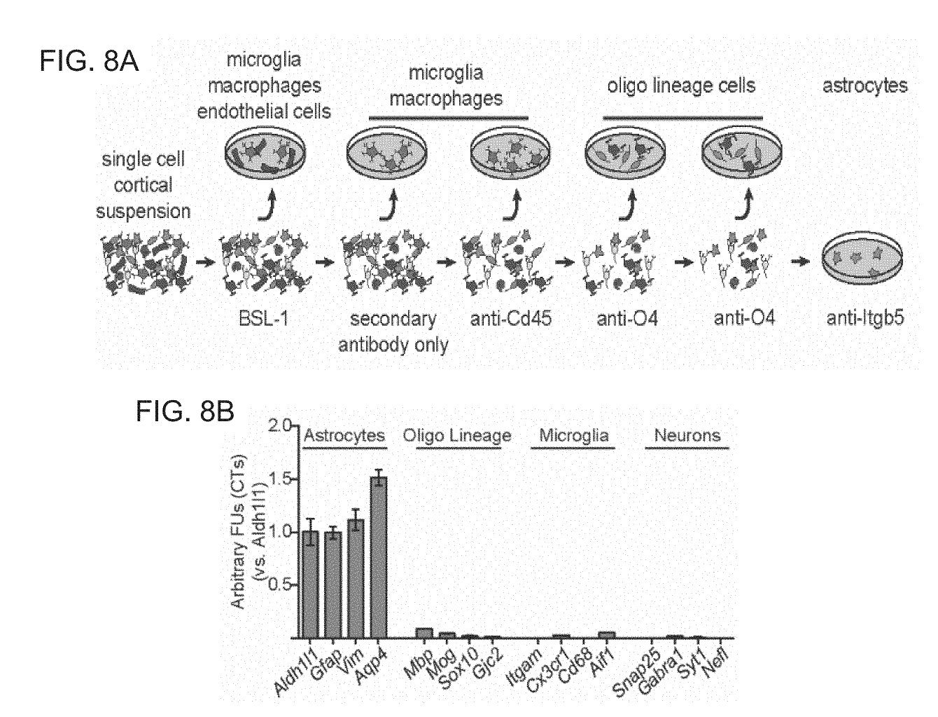

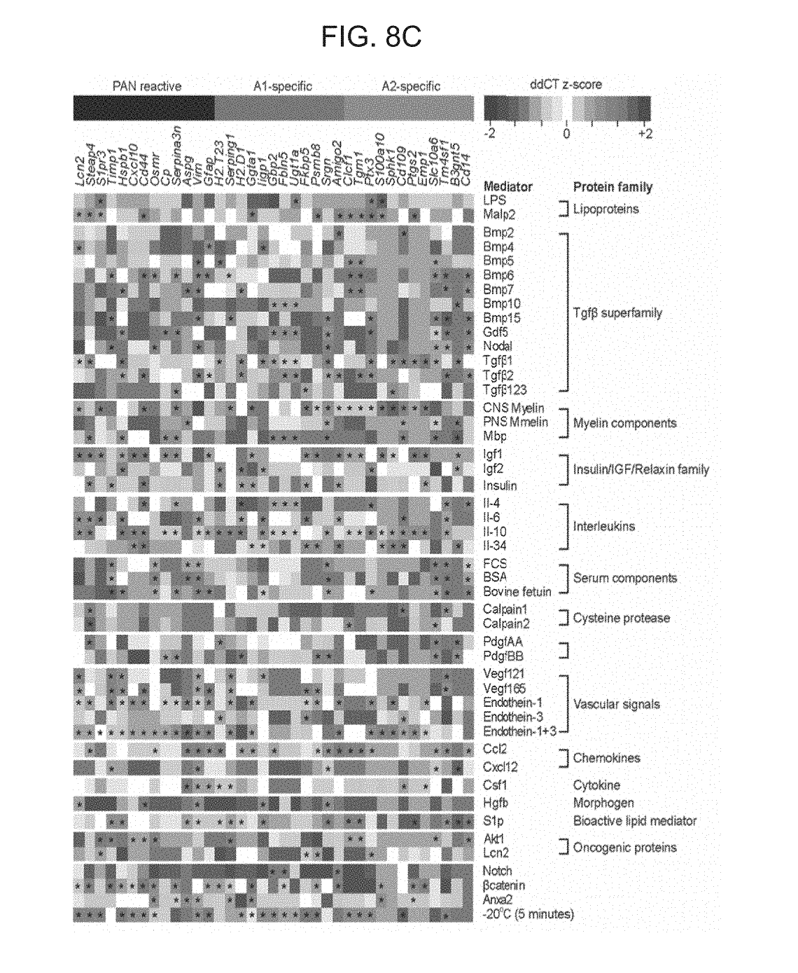

To determine what secreted molecular signals induce the A1 phenotype, we next performed a screen to individually test various candidate molecules that have been implicated previously in reactive astrocyte induction. We used immunopanning to prepare highly pure populations of resting (non-reactive) astrocytes with less than 1% contamination from other central CNS cell types (FIG. 8A-8B), as we described previously¹⁷. We cultured purified astrocytes in serum-free conditions and tested effects of various signaling molecules on gene expression using the microfluidic assay described above. As a control, we first investigated if astrocytes in culture can respond to LPS (or macrophage-activating lipopeptide-2 (Malp2) another lipoprotein that causes neuroinflammation), and found that they do not (FIG. 8A-8C). This was expected as rodent astrocytes lack the receptors and downstream signaling components required for LPS-activation (TLR4 and MYD88)¹³⁻¹⁵. We found however, that several cytokines were able to induce some, but not all, A1 reactive genes. Our best inducers of a partial A1 reactive astrocyte phenotype were interleukin 1 alpha (II-1α), which induced about 70% of A1 genes, tumor necrosis factor alpha (TNFα), which induced about 25% of A1 genes, and complement component 1, q subcomponent (C1q), which induced only a few percent of A1 genes (FIG. 1A). Remarkably, when purified astrocytes were cultured in the presence of all three of these cytokines, astrocytes exhibited an A1 phenotype nearly identical to the A1 phenotype induced by LPS in vivo (FIG. 1A). All three of these cytokines are highly expressed specifically by microglia^{14,16} again pointing to a critical role for microglia in inducing A1 reactive astrocytes.

### Reactive Microglia Induce A1 Reactive Astrocytes by Secreting II-1α, TNFα and C1q

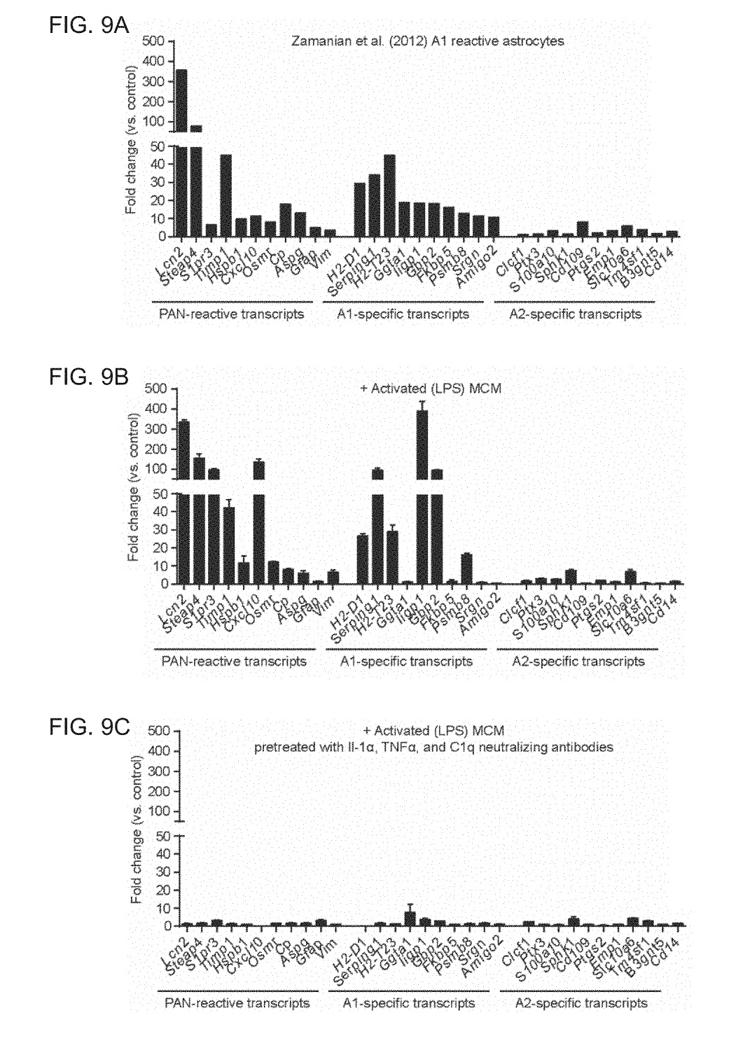

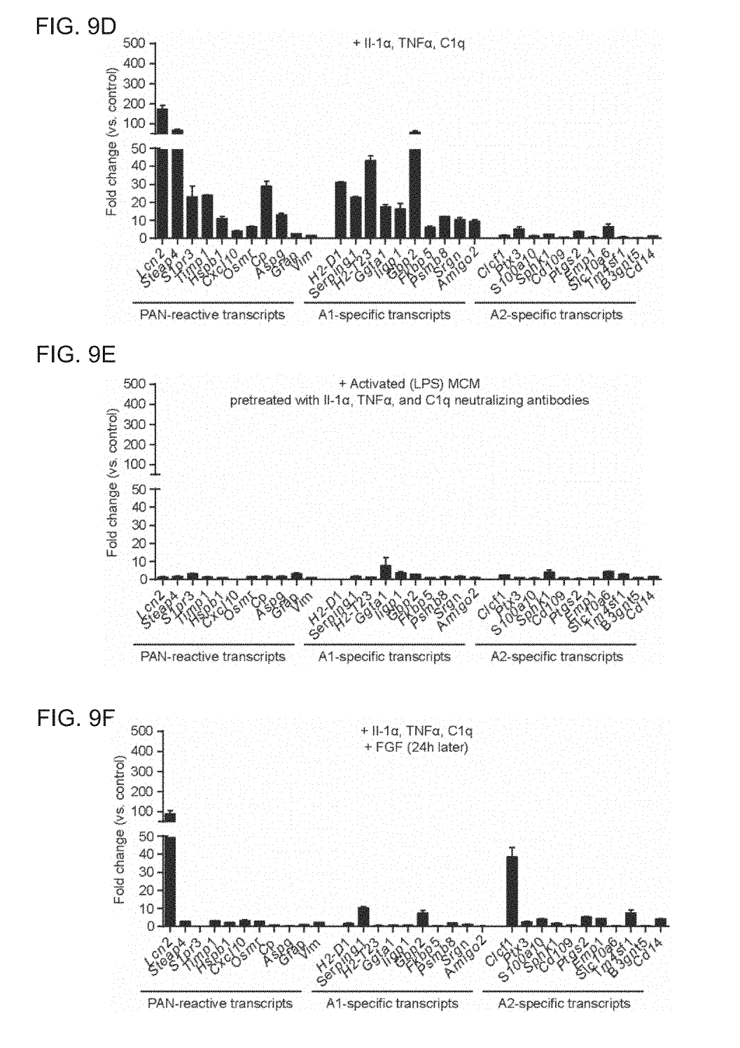

To further confirm microglia are able to induce A1 reactive astrocytes, we purified microglia by immunopanning and cultured purified astrocytes in either control microglia conditioned medium (MCM) or MCM from microglia that had first been made reactive by incubation with LPS for 24 hours. LPS-activated MOM, but not resting MOM, strongly induced expression of A1-specific and PAN reactive genes, while not upregulating expression of A2-specific transcripts (FIG. 1A). The level to which these transcripts were induced was comparable to that seen in vivo following systemic LPS injection⁵ (see FIG. 9A-9F).

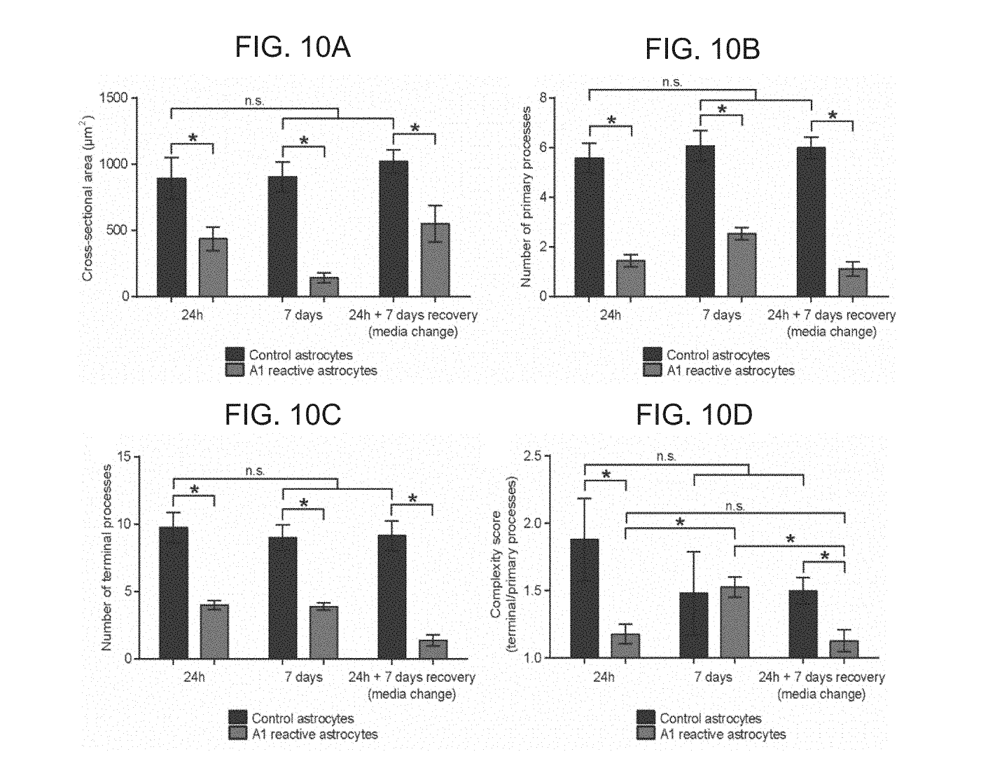

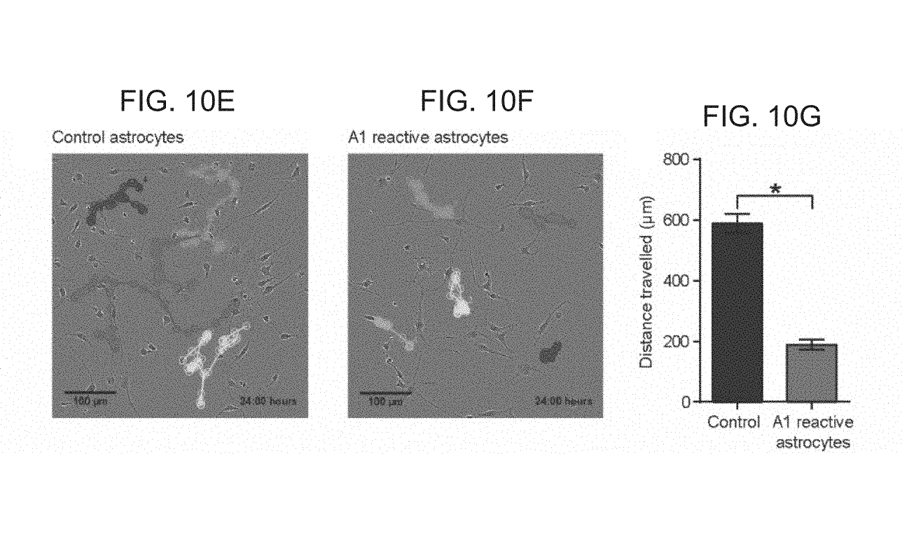

To verify which cytokines LPS-activated microglia use to signal A1 induction, we purified microglia by immunopanning, and used cytokine antibody arrays (FIG. 1B) and western blot analysis (FIG. 10) to determine which cytokines are secreted by resting and LPS-activated microglia after 24 hours of culture. Levels of II-1α, TNFα and C1q were all significantly elevated after microglial activation (FIG. 1A-1B). II-1β secretion also increased in the LPS-activated MOM, but II-1β was unable to induce expression of A1 transcripts (FIG. 1A). We also tested a range of other microglia secreted cytokines that were unable to induce A1s (FIA. 8A-8C). The combination of II-1α, TNFα and C1q, however, closely mimicked that of LPS-reactive MOM inducing the expression of A1-specific and PAN-reactive genes, while not upregulating expression of A2-specific transcripts (FIG. 1A, FIG. 9A-9F).

To ensure that no other factors secreted by LPS-activated microglia besides II-1α, TNFα and C1q could also make resting astrocytes reactive, we collected LPS-activated MOM and pre-treated it with neutralizing antibodies to II-1α, TNFα, and 01 q. This pre-treated MOM was unable to induce reactive astrocyte genes when it was incubated with resting astrocytes for 24 hours (FIG. 1A, FIG. 9E). Thus II-1α, TNFα and C1q together are sufficient to induce the A1 phenotype, and are necessary for LPS-reactive microglia to induce A1s in vitro.

To investigate whether cessation of II-1α, TNFα, and C1q signaling enables A1 reactive astrocytes in vitro to revert back to resting astrocytes or whether the A1 phenotype, once induced, is relatively stable, we removed all three cytokines from pure A1 reactive astrocyte cultures, and added neutralizing antibodies to all three to make sure they were fully inhibited. After 7 days, we assessed levels of A1 transcripts and found the A1 phenotype had not reverted. As a proof of principal, we also investigated if additional molecules could revert A1s to a non-reactive phenotype. We tested the anti-inflammatory cytokine TGFβ and FGF (as it has been previously shown that astrocyte activation is suppressed in the injured brain by FGF signaling¹⁸). We grew A1s in culture, then used 24 h treatment with TGFβ or FGF and found that both significantly decreased reactive astrocyte transcript levels (FIG. 1D, FIG. 9A-9F).

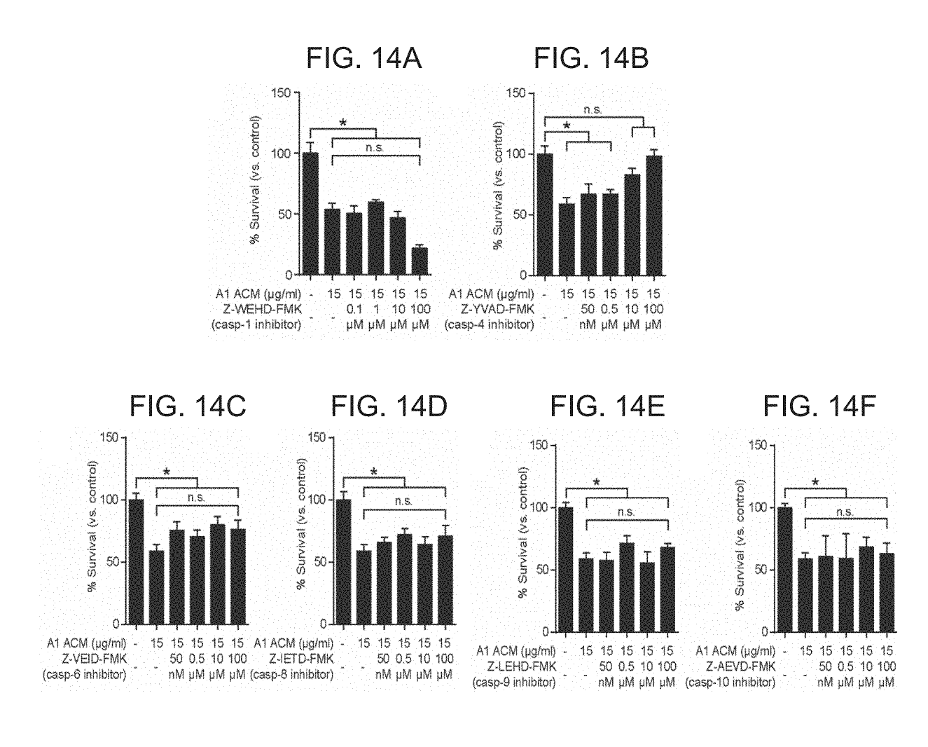

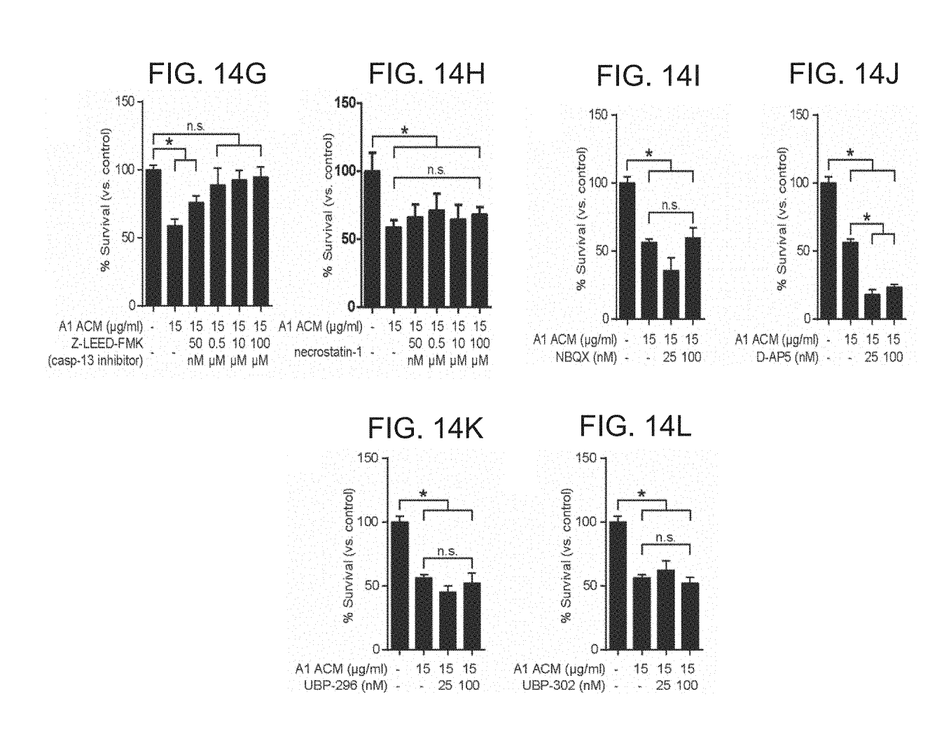

As microglia are required to induce A1 reactive astrocytes and microglia induce A1s by secretion of II-1α, TNFα, or C1q, we next investigated if genetic deficiency of these cytokines would be sufficient to prevent A1 astrocyte reactivity in vivo. First we checked if single knock out mice for II-1α, TNFα, or C1q were still able to produce neuroinflammatory reactive microglia following systemic LPS injection. Using qPCR we saw that microglia collected from II-1α^{-/-}, TNFα^{-/-}, and C1q^{-/-} animals still had many reactive transcripts highly upregulated 24 h following LPS injection (as determined from highly purified microglia¹⁶, FIG. 14A-14L). We next used astrocytes purified from these same mice and used our microfluidic qPCR screen to determine whether they were reactive. Each of the knock-out mice had significantly decreased A1 astrocyte reactivity (FIG. 1E) with astrocytes from Tnfα^{-/-} mice failing to upregulate expression of most reactive transcripts, followed by and then C1q^{-/-} single knock-out mice (FIG. 1F). Additionally, we looked at double knock-out mice (II-1α^{-/-}TNFα^{-/-}, II-1α^{-/-}C1q^{-/-}, TNFα^{-/-}C1q^{-/-}) and triple knock-out mice (II-1α^{-/-}TNFα^{-/-}C1q^{-/-}) and saw similar decreases in A1 reactivity, with triple knock-out animals having no response following systemic LPS injection (FIG. 1A-1H). Taken together our data show that microglia derived II-1α, TNFα, and C1q work together to mediate A1 astrocyte reactivity phenotype following LPS-induced neuroinflammation.

### A1 Reactive Astrocyte Lose Polarity

To find out whether our new in vitro model of A1 reactive astrocytes demonstrates alterations in astrocytes following injury/trauma we first investigated their morphology. Untreated, purified, serum-free cultured astrocytes contain many primary processes radiating from their cell somas, which branch multiple times (FIG. 1F, FIG. 10A-10G). GFAP immunostaining showed normal cellular localization, and AQP4 protein was localized to distal processes (FIG. 1F). In contrast, A1 reactive astrocytes in culture lost their fine processes and AQP4 immunoreactivity was diffuse, covering the entire cell rather than just process terminations (FIG. 1F, FIG. 10A-10G). A1 reactive astrocytes had a 3-fold increase in GFAP protein levels compared to control astrocytes as measure by western blotting (FIG. 1G). The surface area of these cells (when measure in GFAP-stained images) was decreased by about 50% at 24 h (FIG. 1H). Thus A1 astrocyte reactivity in vitro is accompanied by a decrease in processes number and complexity, an increase in GFAP levels, and a loss of polarization of AQP4 localization (as reported in vivo¹⁹).

A1 Reactive Astrocytes have Decreased Synaptic Functions

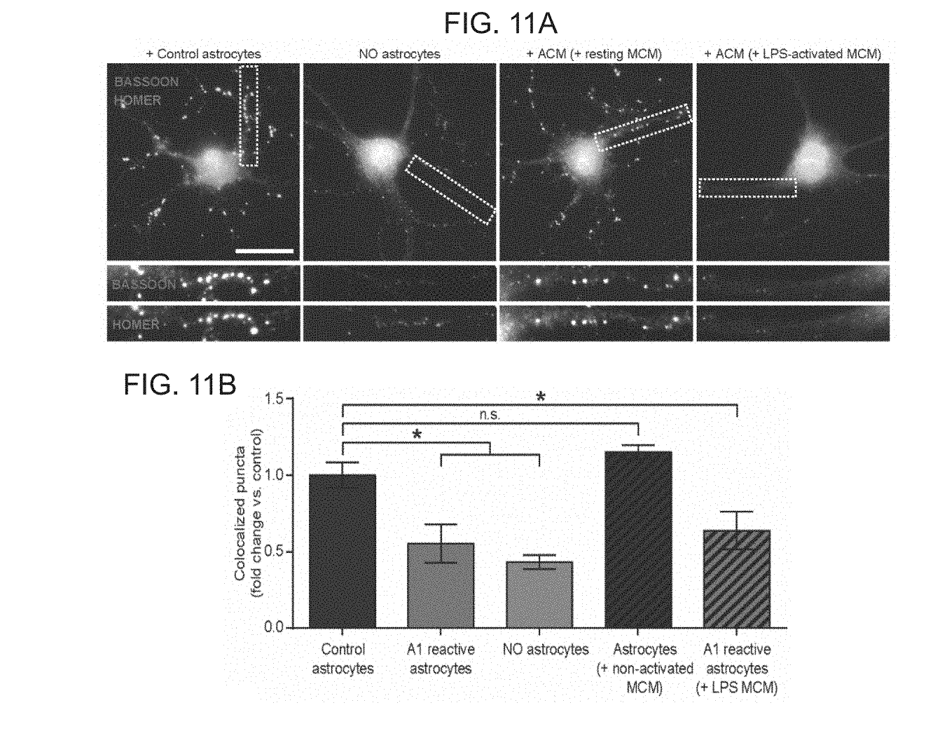

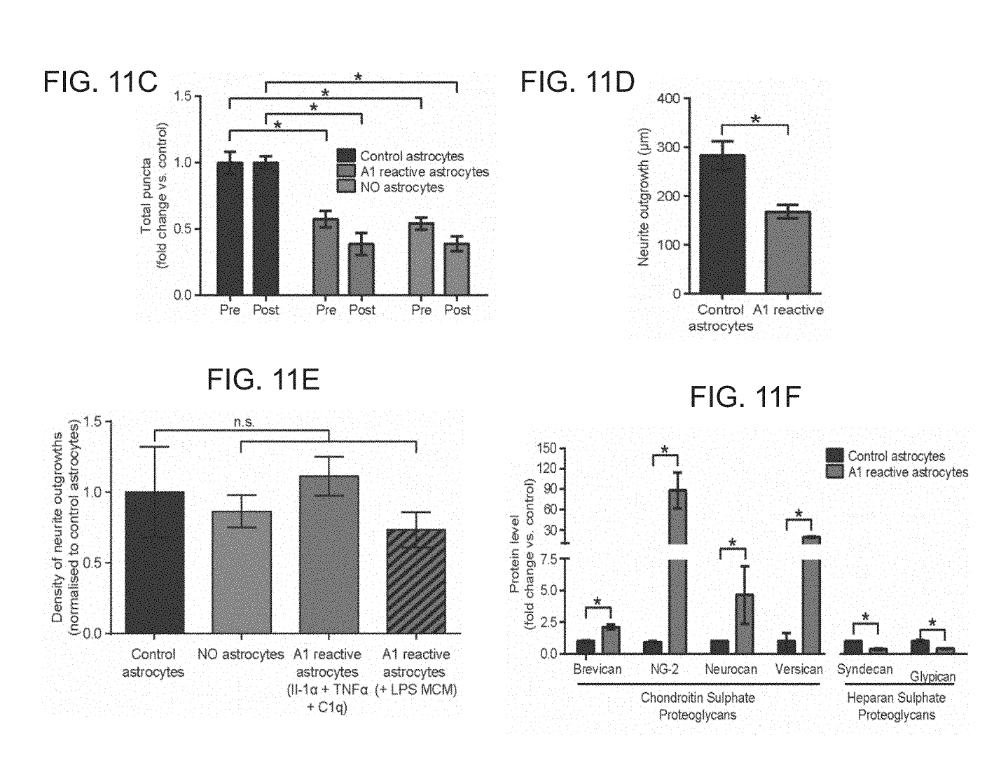

We next investigated whether A1 reactive astrocytes can induce formation of functional synapses in vitro. We cultured purified retinal ganglion cells (RGCs) with low numbers of resting or A1 reactive astrocytes and after one week quantified synapse number by double immunostaining for pre- and post-synaptic proteins BASSOON and HOMER (FIG. 2A, FIG. 11A-11B). RGCs cultured with reactive astrocytes had a 50% decrease in synapse number compared to those grown with control astrocytes (FIG. 2B). This decrease in synapse number was due to a decrease in both pre- and post-synaptic puncta (FIG. 11C), and not simply a failure of the pre- and post-synaptic elements to colocalize. It could also not be explained by decreased neurite density, as neurites close to the RGC soma where synapse number was counted were not decreased in presence of A1s (FIG. 11E). When RGCs were cultured with control astrocytes to induce synapse formation and then cultured with A1 reactive astrocyte, synapse number significantly decreased by about 40% suggesting that A1 reactive astrocytes are either unable to maintain these functional synapses or are actively able to disassemble them.

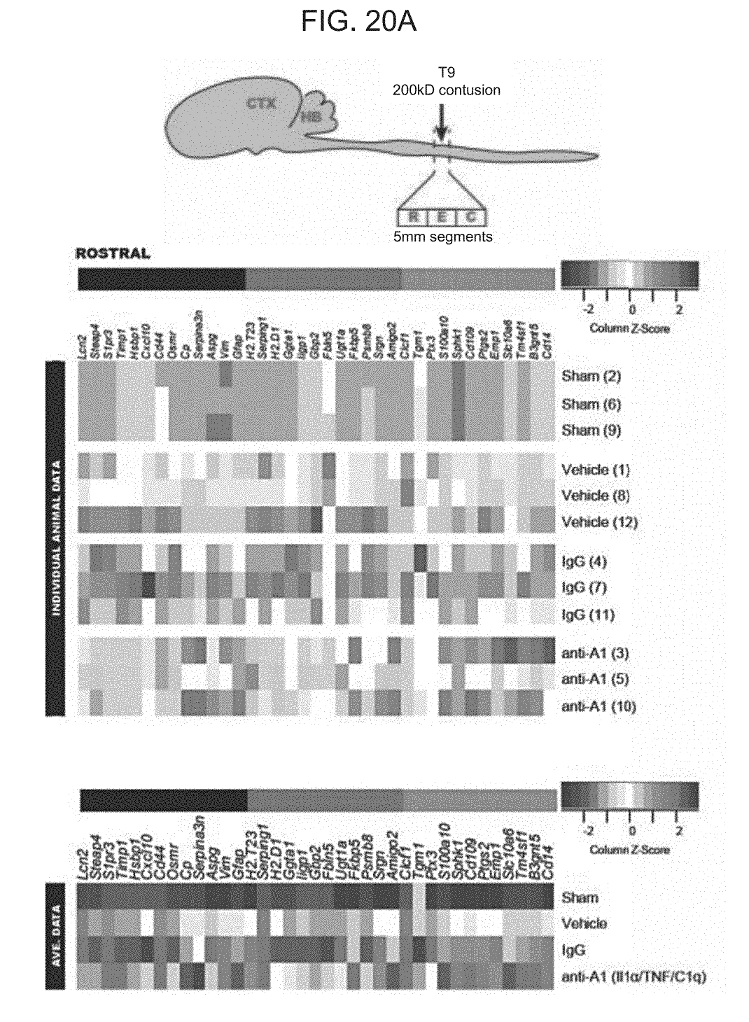

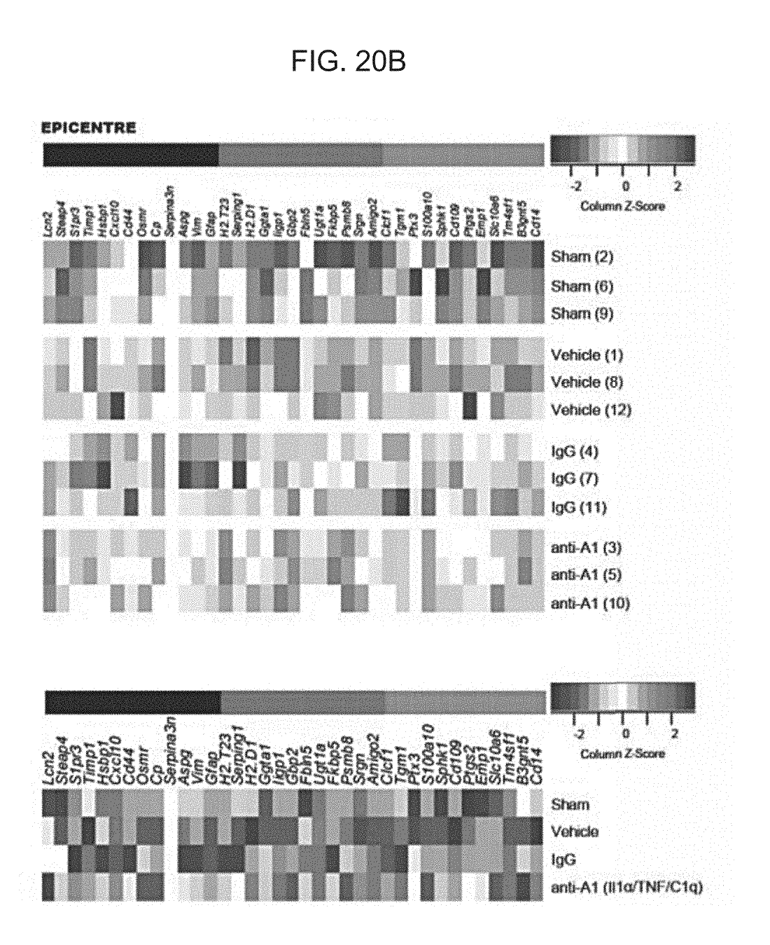

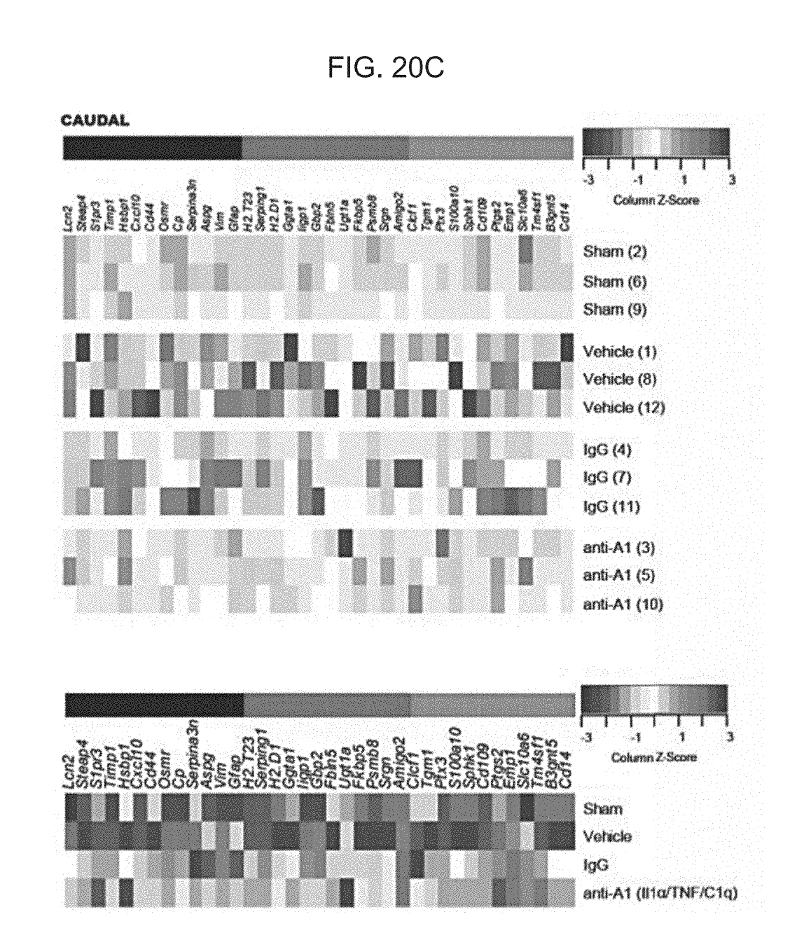

Astrocytes induce formation of excitatory synapses by secreting GPCG4/6²⁰, SPARCL1²¹, and thrombospondins (THBS1/2)²², so we next investigated whether reactive astrocytes still produce these factors. Quantitative PCR showed decreased expression levels of Gpc6 and Sparcl1, while simultaneously showing increased expression of thrombospondins Thbs1 and Thbs2 (FIG. 20). Gpc4, showed no change in levels (FIG. 20). This large increase in thrombospondins (which should increase synaptic number) suggests that the decreased synapse number may reflect an active A1-induced toxicity to synapses (see below). To determine effects of A1 reactive astrocytes on the number of functional synapses we used whole-cell patch clamp recording on RGCs cultured with resting astrocytes or A1 s. RGCs cultured with A1s had significantly decreased frequency and amplitude of miniature excitatory postsynaptic currents (mEPSC) when compared to RGCs cultured with resting astrocytes (FIG. 2D-2G). Taken together these results show that, A1 reactive astrocytes induce the formation of fewer synapses, and the few synapses that they do induce are significantly weaker when compared to those produced in the presence of healthy resting astrocytes.

A1 Reactive Astrocytes have Significantly Decreased Phagocytic Capacity

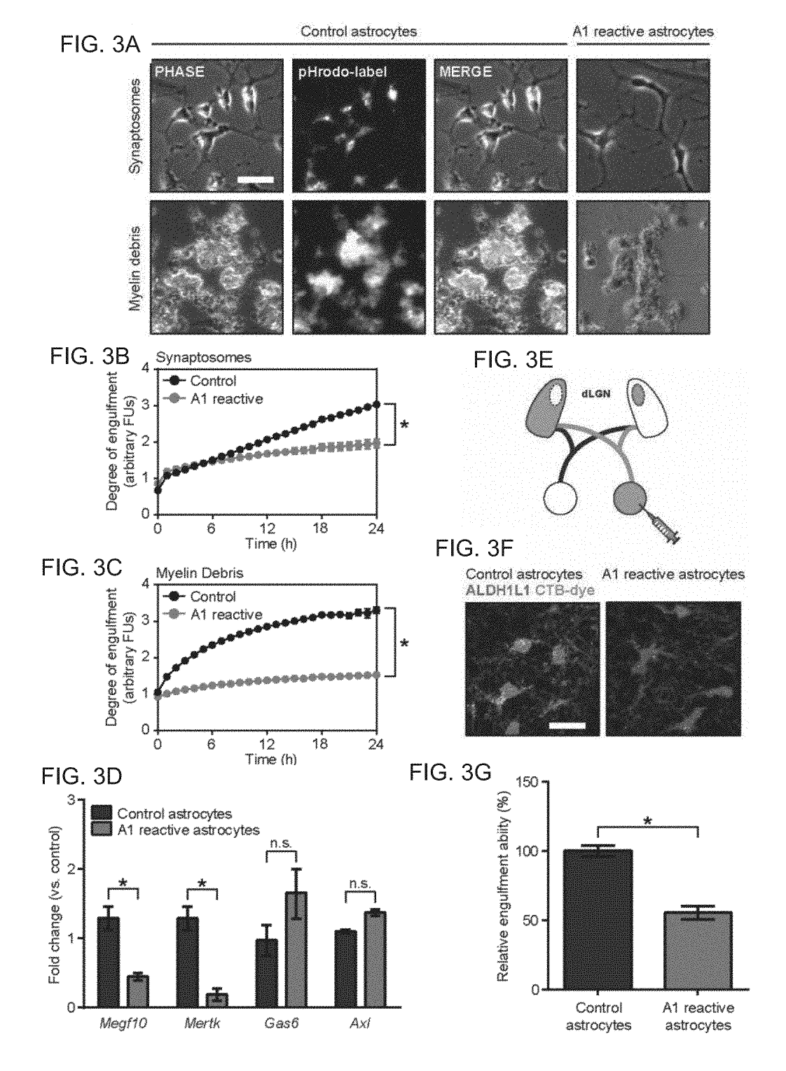

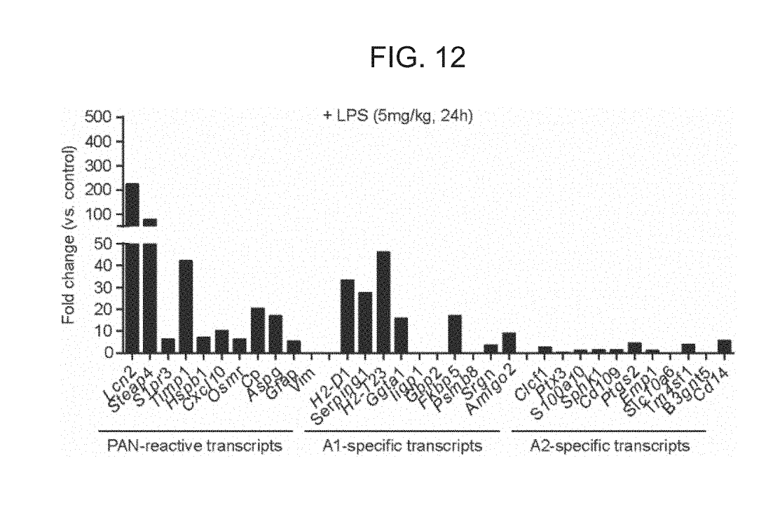

To compare phagocytic ability of normal astrocytes and A1 reactive astrocytes, we measured their engulfment of purified synaptosomes conjugated with pHrodo (a pH-sensitive dye that only fluoresces when present in lysosomes after engulfment by cells). A1 reactive astrocytes engulfed 50 to 75% fewer synaptosomes than control astrocytes (FIG. 3A-3B). Similarly we found that control astrocytes are able to robustly phagocytose myelin debris, but upon conversion to an A1 reactive phenotype they almost completely lose this capacity (FIG. 3A, FIG. 30). This phagocytic deficit corresponded with a 90% decrease in Mertk and a 60% decrease in Megf10 mRNA, the phagocytic receptors that we have previously found mediate synaptic phagocytosis³, with no change in expression of bridging molecules Gash and Axl (FIG. 3D). To determine whether A1s also display decreased phagocytic ability in vivo, we used Aldh1I1-eGFP transgenic mice to visualize phagocytosis of synapses by astrocytes in vivo. We first checked that we could induce A1 reactivity in astrocytes in the lateral geniculate nucleus (LGN) of P3 Aldh1I1-eGFP mice by systemic injection of LPS, collecting mRNA from the LGN 24 hours later, and performing microfluidic qPCR analysis. We found that astrocytes in the LGN of P3 mice are strongly polarized to an A1 reactive phenotype when treated systemically with LPS (FIG. 12). We then induced this A1 astrocyte reactivity in the LGN of another cohort of mice in which we preformed intravitreal injections of an Alexa594-conjugated anterograde tracer cholera toxin-β subunit, CTB-594, at P4 to label the pre-synaptic side of synapses in the LGN (FIG. 3E). Twenty four hours later, confocal microscopy was used to reconstruct the entire LGN and we used an image-processing algorithm to measure the volume of engulfed CTB-labelled synapses inside Aldh111-eGFP fluorescent astrocytes as we previously reported³. We found that A1 reactive astrocytes in the LGN in vivo show the same significant loss of synaptic engulfment ability (around 50% compared to astrocytes in saline-treated control animals) as was seen in our in vitro assay (see above, FIG. 3F-3G). Combined these data show that A1 reactive astrocytes have deficiencies in phagocytosis of both synaptosomes and myelin debris in culture, that this deficiency can also influence the efficiency of synaptic pruning in vivo, and suggest that A1 reactive astrocytes might well lose the capacity to clear myelin debris in vivo, an important area for future investigation.

### A1 Reactive Astrocytes are Neurotoxic

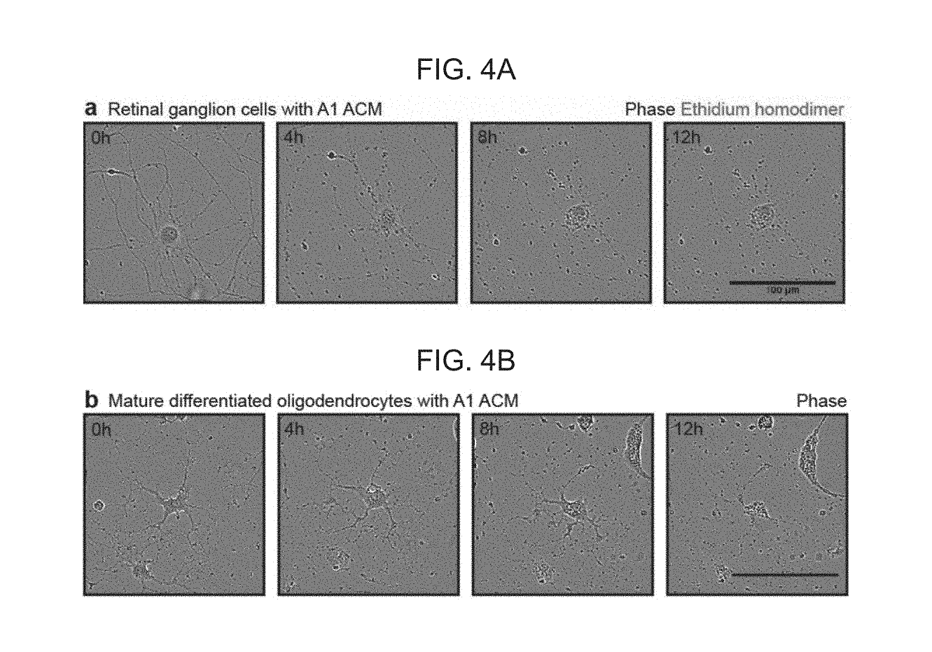

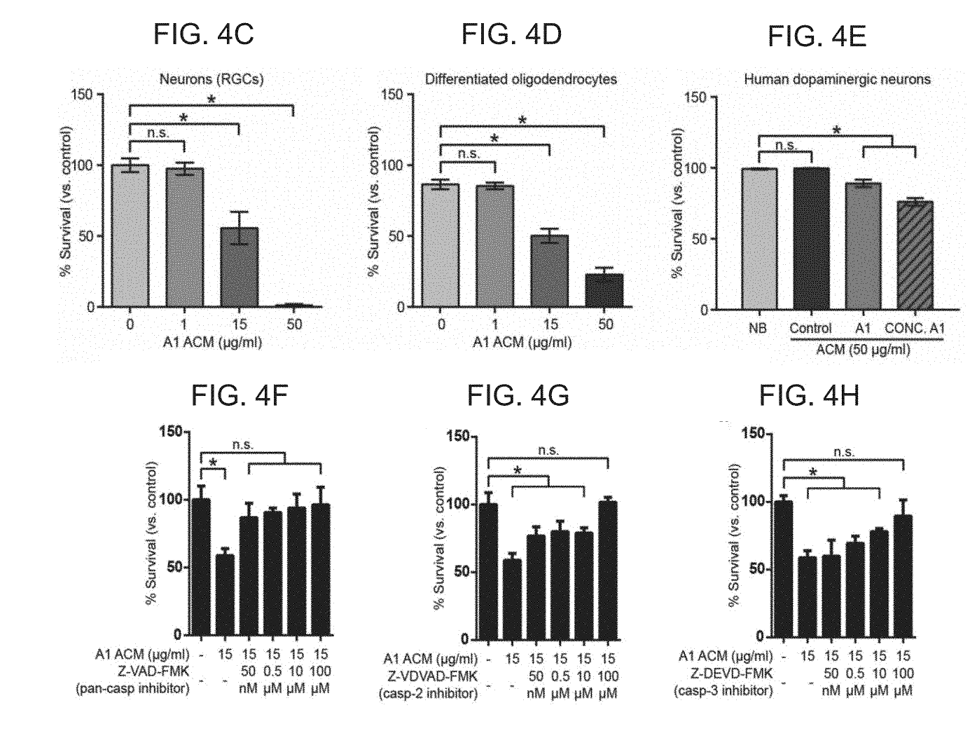

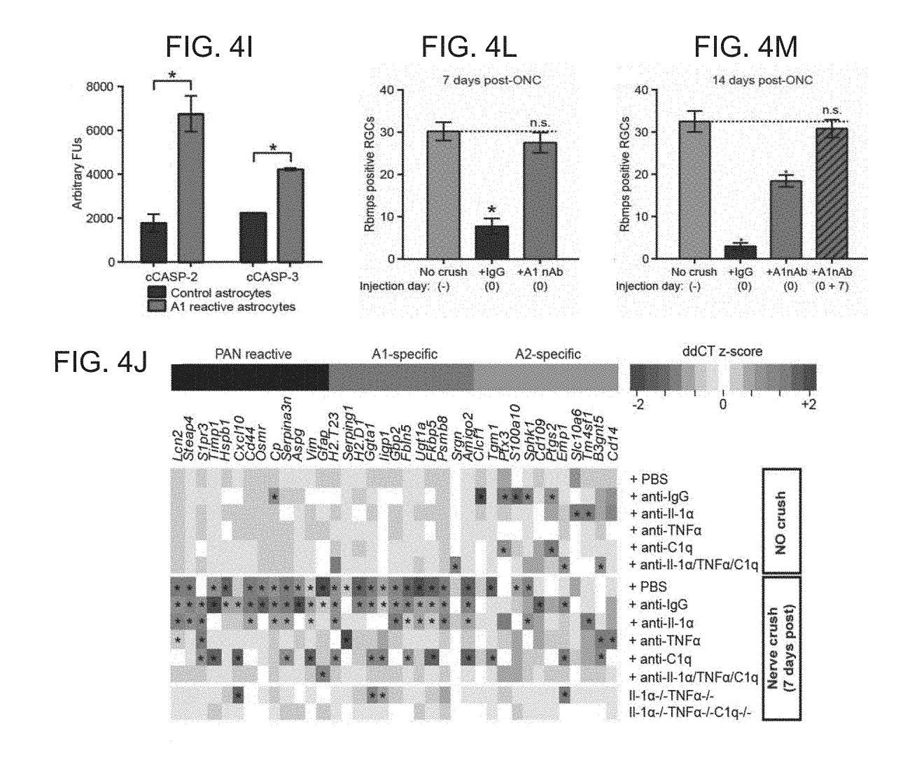

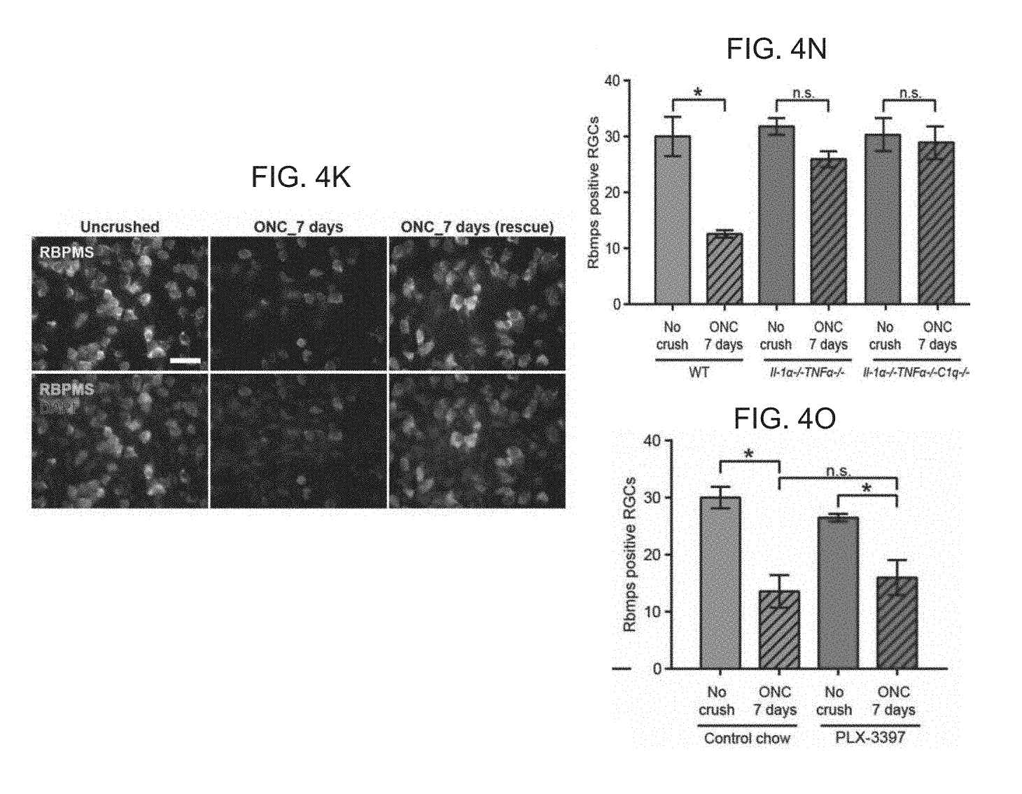

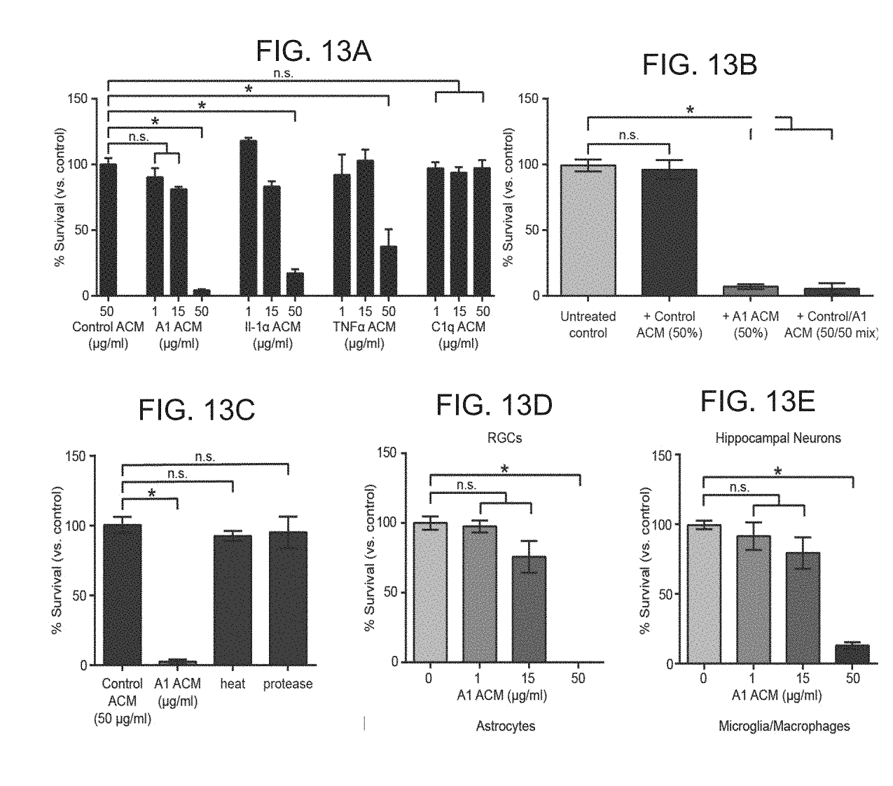

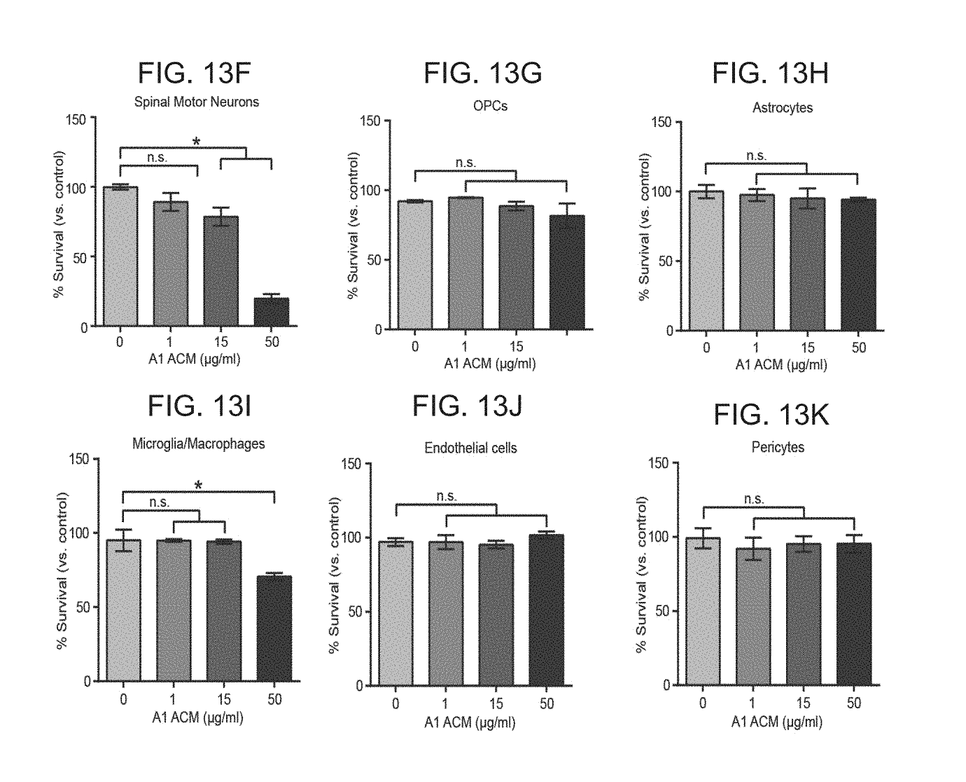

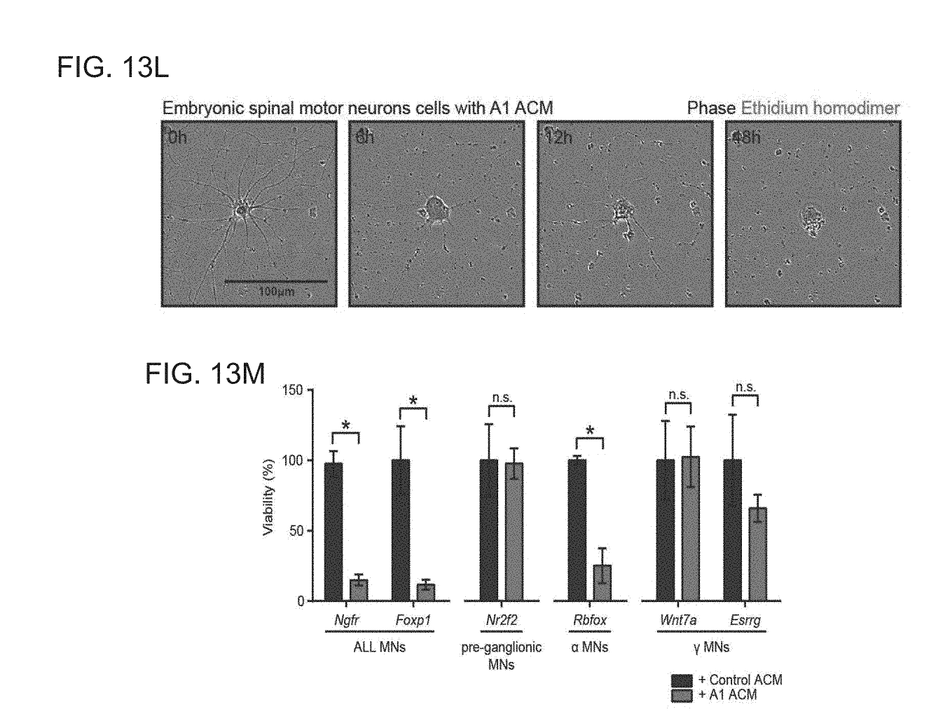

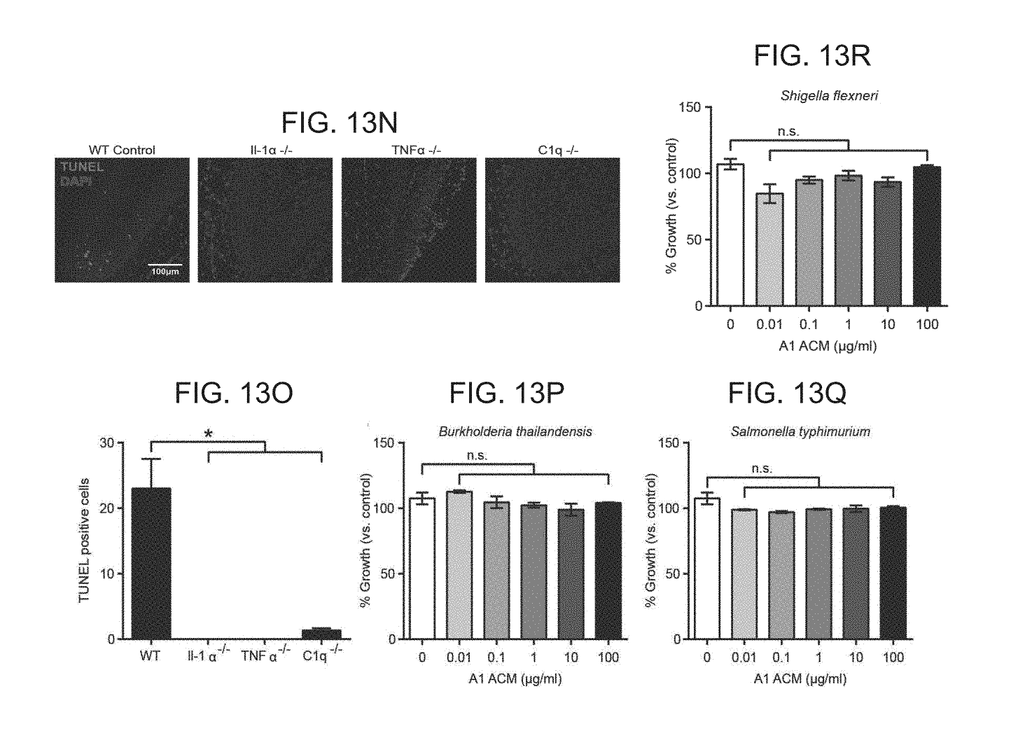

Normally astrocytes promote CNS neuronal survival²³. To determine whether A1 reactive astrocytes also promote neuronal survival, we co-cultured control and A1 reactive astrocytes with purified RGCs and measured their viability after 24 h. We found that RGCs rapidly died when grown with increasing concentrations of A1 reactive astrocyte conditioned media (FIG. 4A-4B). This process began with rapid process retraction and blebbing occurring by 4 h with membrane breakdown at 6-8 h followed by death, as shown by entry of the fluorescent cell-impermeant viability indicator ethidium homodimer-1. At the highest concentrations there was almost 100% death of cells (FIG. 4A-4B). To determine if A1s also induced death of other CNS cell types, we treated other purified CNS cell types in culture with increasing concentrations of A1 astrocyte conditioned medium (ACM). A1s also induced death of mature oligodendrocytes, but did not kill oligodendrocyte precursor cells (OPCs), astrocytes, pericytes, endothelial cells or microglia (FIG. 4C, FIG. 13G-13K). A1s were similarly toxic to cortical neurons and embryonic spinal motor neurons (FIG. 13D-13F, FIG. 13L-13M), however even at high doses spinal motor neurons were still around 20% viable. We then investigated if this 20% of surviving motor neurons represented a specific subset of neurons and found that it is preganglionic and gamma motor neurons are not susceptible to A1-induced toxicity (FIG. 13M). This death could not be attributed to II-1α, TNFα, and C1q which did not cause death of cells in purified cultures (data not shown). Thus, A1s secrete a soluble toxin that rapidly kills CNS neurons and mature oligodendrocytes, but not other CNS cell types.

Are II-1α, TNFα, or C1q needed together to induce astrocytes to secrete this toxin? To find out, we prepared conditioned media from purified astrocytes treated individually with II-1α, TNFα, or C1q and added it to purified cultures of RGCs, and measured their viability at 24 h. Only ACM from astrocytes treated with either II-1α or TNFα was toxic, but this effect was not as great as that seen when treating with full A1 ACM at the same concentration (FIG. 13A).

We next used pharmacological blockers to determine if A1s induce neuronal death by excitotoxicity, necroptosis, or apoptosis. Neuronal death was not due to glutamate excitotoxicity because it could not be prevented by the presence of NMDA, AMPA, or kainate receptor blockers, and was not due to necroptosis as it could not be prevented by necrostatin (FIG. 14A-14L). In contrast, death of RGCs and oligodendrocytes could both be prevented by caspase 2 and 3 inhibitors, which block apoptosis (FIG. 4D-4F)). Blocking other caspases did not preserve neuron and oligodendrocyte viability (FIG. 14A-14L). In addition, there was a significant increase in cleaved caspase 2 and 3, as measured by western blotting from RGCs and oligodendrocytes treated with A1 reactive toxic media (FIG. 4G). Thus, A1s induced death of neurons and oligodendrocytes is most likely by apoptosis.

Apoptosis is typically caused by loss of neurotrophic support raising the possibility that A1 s were not inducing death by toxicity but rather by failing to secrete their normal neurotrophic factors. The rapid time course of death within hours was far faster than that we observed by withdrawing neurotrophic support, which typically takes several days, consistent with the possibility of toxicity. To directly test whether the death was caused by toxicity, we cultured RGCs and oligodendrocytes with a 50/50 mix of both control astrocyte and A1 reactive ACM. In 50% control ACM only, the cells remained viable, but the addition of 50% A1 ACM rapidly caused apoptosis (see FIG. 13A-13R). Biochemical characterization demonstrates that at least a portion of the neurotoxic effect is proteinaceous, at the effect is heat and protease sensitive and the neurotoxin is greater than 30 kD (FIG. 13C).

As LPS injection has been used as a model of dopaminergic neuronal cell death in vivo in the dentate gyrus/hippocampus, we performed LPS injections in wild type and II1α^{-/-}, TNFα^{-/-}, or C1q^{-/-} individual knockout animals, followed by TUNEL labelling to visualize apoptotic cells. Single knock-out animals were sufficient to decrease the number of TUNEL+, apoptotic cells, in the dentate gyrus (FIG. 13A-13R). In addition we tested the susceptibility of human dopaminergic neurons to A1-induced toxicity in vitro and found that like rodent cells, they also showed decreased viability after incubation with A1 ACM (FIG. 4N).

It is surprising that reactive astrocytes secrete a toxin, but we hypothesized that since the gram negative bacterial cell wall derived LPS is a strong inducer of A1 s, that the toxin might also kill bacteria. We treated three gram negative bacteria types: Salmonella typhimurium, Burkholderia thailandensis. and Shigella flexneri, with A1 ACM and checked for growth at 16 h. We saw no decrease in the proliferation of any bacterial strain (FIG. 13P-13R)—suggesting that A1 ACM is not directly toxic to invading gram negative bacteria.

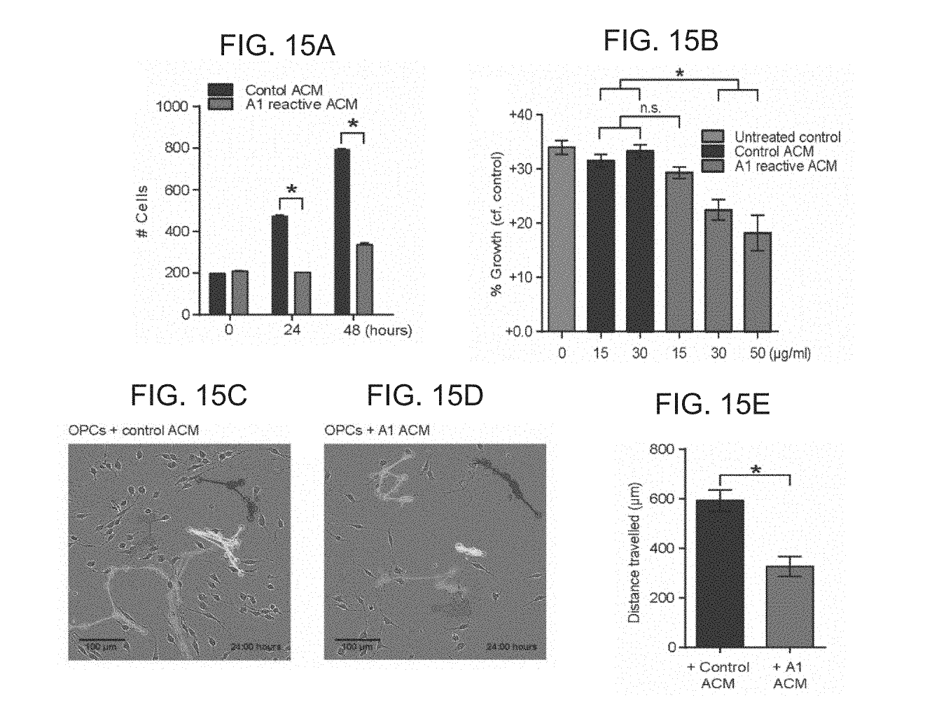

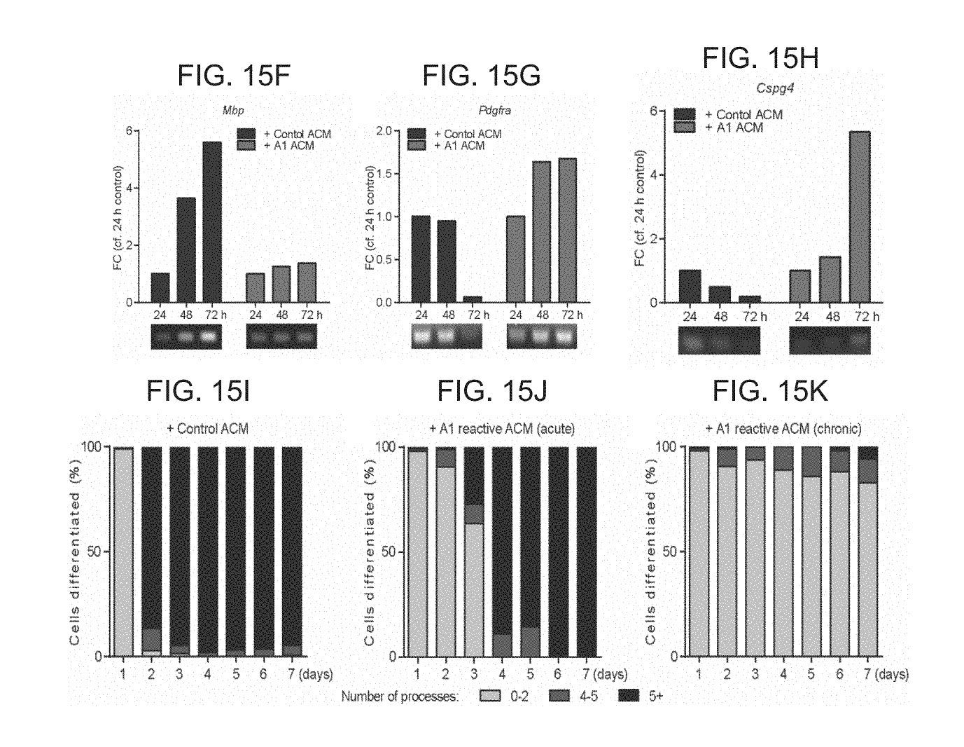

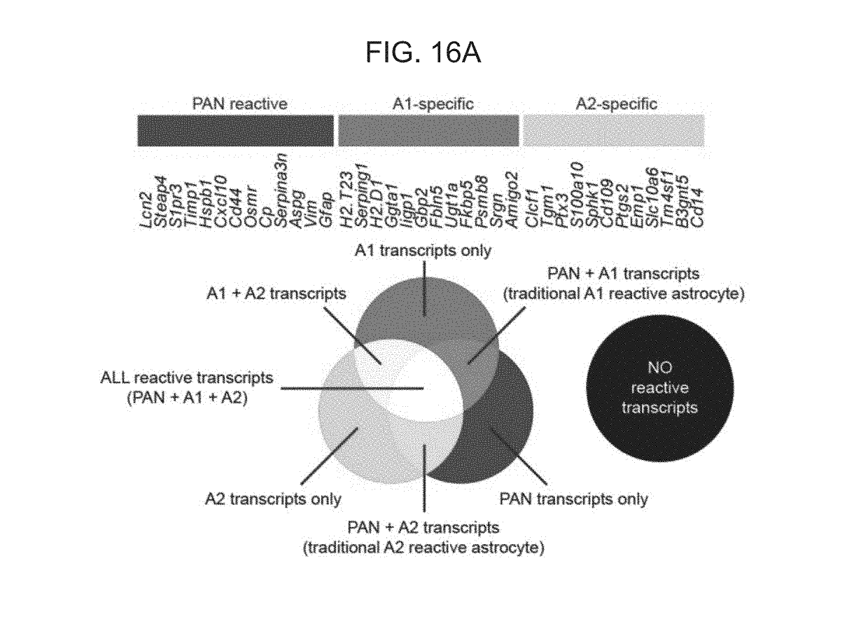

As A1s are toxic to mature oligodendrocytes (OLs) but not OPCs, we investigated whether they may impair OPC division, differentiation, and migration by culturing OPCs in an incubator while observing them with time-lapse microscopy. By using cell proliferation assays and tracking movement using ImageJ we found that A1 ACM significantly decreased division compared to cells treated with control ACM (FIGS. 15A-15B), and A1 ACM-treated OPCs migrated at only about half the rate of OPCs treated with control ACM (FIG. 15C-15E). To find out whether A1 ACM affected differentiation of OPCs into mature OLs, we cultured OPCs in differentiation media and treated them with either control or A1 ACM, and used RT-PCR to detect expression levels of the mature OL marker Mbp and the OPC markers Pdgfra and Cspg4, While OPCs cultured with control ACM rapidly differentiated into Mbp-expressing mature OLs, A1 ACM strongly inhibited differentiation as Mbp expression did not increase, Pdgfra and Cspg4 expression did not decrease and the OPCs did not take on the typical multiple process bearing morphology of OLs (FIG. 15F-15K). Thus in addition to inducing death of mature OLs, A1 reactive astrocytes strongly inhibit proliferation, migration, and differentiation of OPCs.

A1 Reactive Astrocyte Formation after Injury In Vivo can be Inhibited Preventing Death of CNS Neurons after Axotomy

It has been a longstanding mystery why CNS but not PNS neurons die after axotomy. One idea has been that axotomy interrupts a retrograde neurotrophic signal, but because injured spinal cords contain neuroinflammatory microglia and/or macrophages²⁴, we hypothesized that axotomy could also induce formation of A1 reactive astrocytes which in turn release their neurotoxin to kill the axotomized neuron. We used optic nerve crush in postnatal rats as a model system. To find out whether A1s were generated in the postnatal day 7 (P7) rat retina following optic nerve crush, we crushed optic nerves and after 7 days used our microfluidic assay to see if A1 genes were upregulated in the retina. Robust A1 generation rapidly occurred after optic nerve crush and was temporally paired with death of RGCs (as counted by staining with the RGC-specific marker RBPMS, FIG. 4H-4J). Injection of neutralizing antibodies to II-1α, TNFα, and C1q together into the vitreous of the eye inhibited A1 formation and prevented death of retinal ganglion cells at 7 days after optic nerve crush (FIG. 4I-4J). We then looked at whether the protective effect of neutralizing antibodies could be observed at a longer time point. At 14 days post optic nerve crush the number of viable RGCs had again decreased, however following a second injection of neutralizing antibodies at 7 days, these cells could be rescued once more (FIG. 4K). Finally, we performed optic nerve crushes in both double (II-1α^{-/-}TNFα⁻¹), and triple knock-out mice (II-1α^{-/-}TNFα^{-/-}C1q^{-/-}) that fail to generate A1 reactivity (FIGS. 1A-1H), and found that 7 days following optic nerve crush RGCs remained viable, unlike their wild-type control counterparts (FIG. 4A-4O). These data provide strong correlative evidence that death of RGCs by apoptosis after axotomy is not due to trophic deprivation but is instead due to release of a toxic signal by nearby neurotoxic A1 reactive astrocytes.

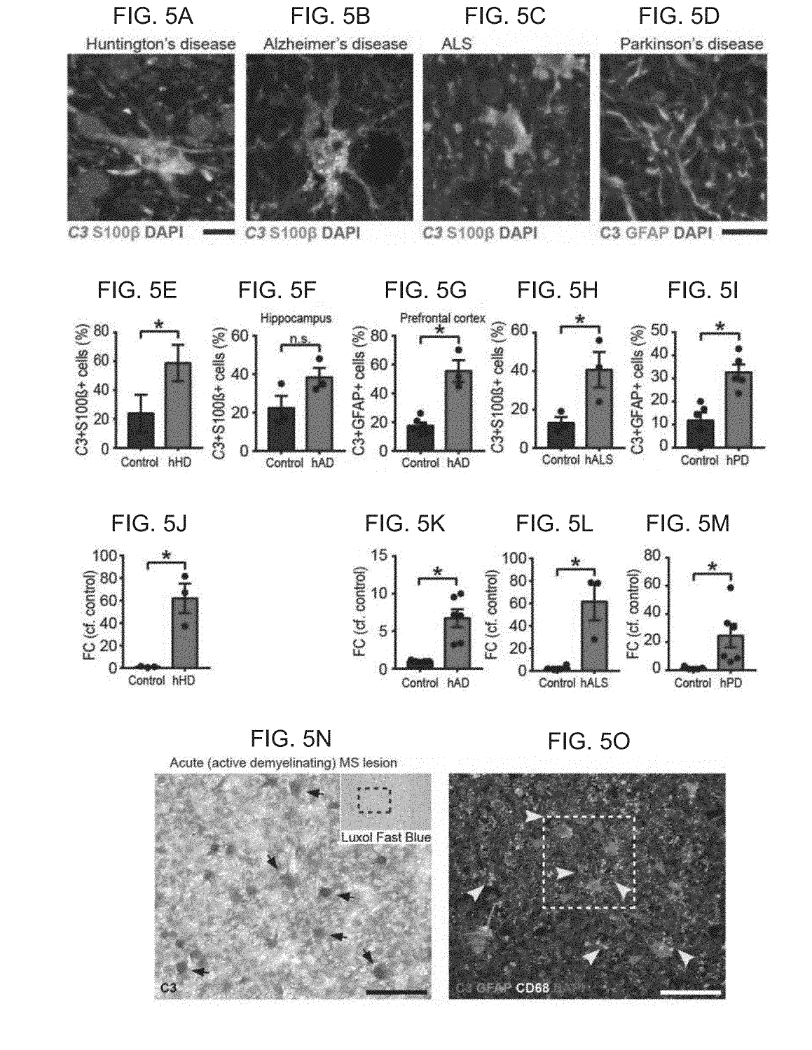

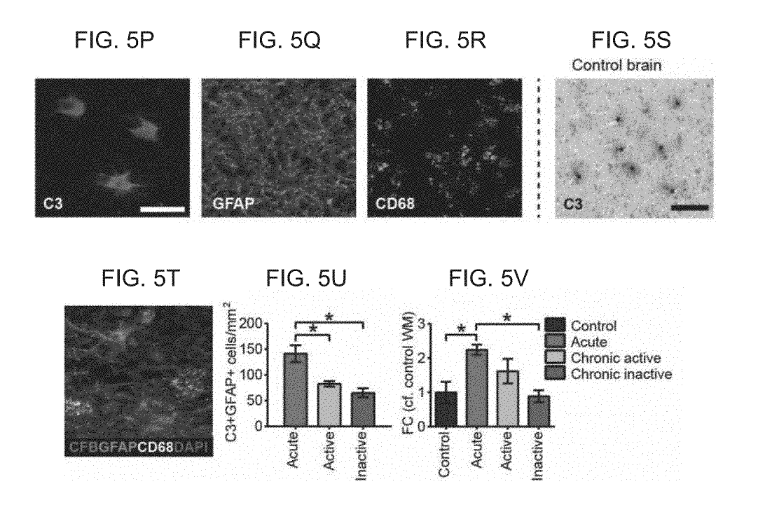

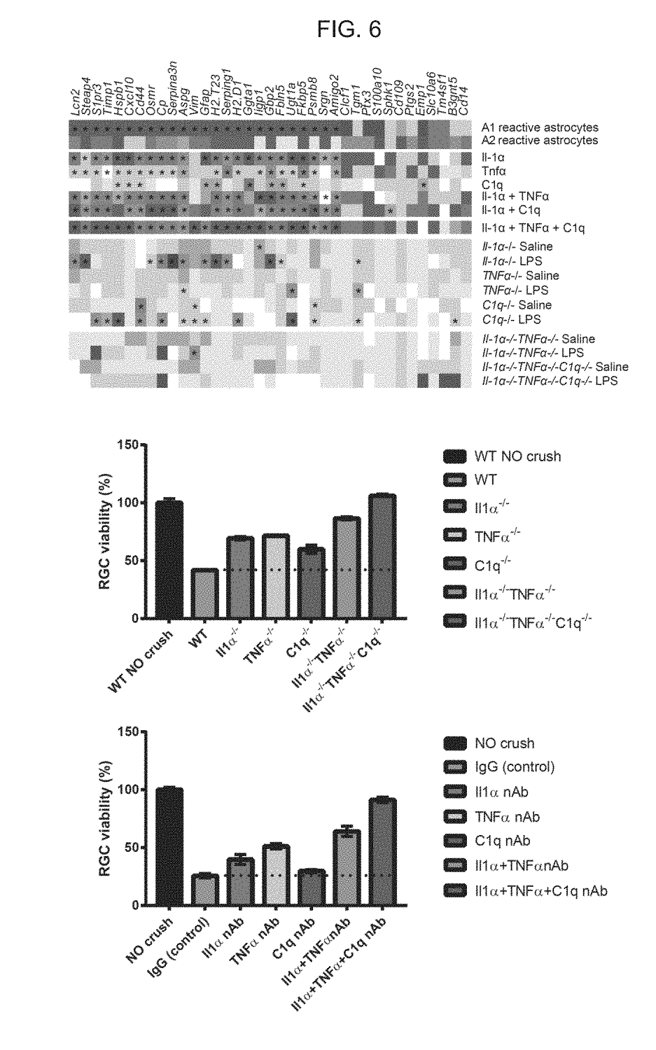

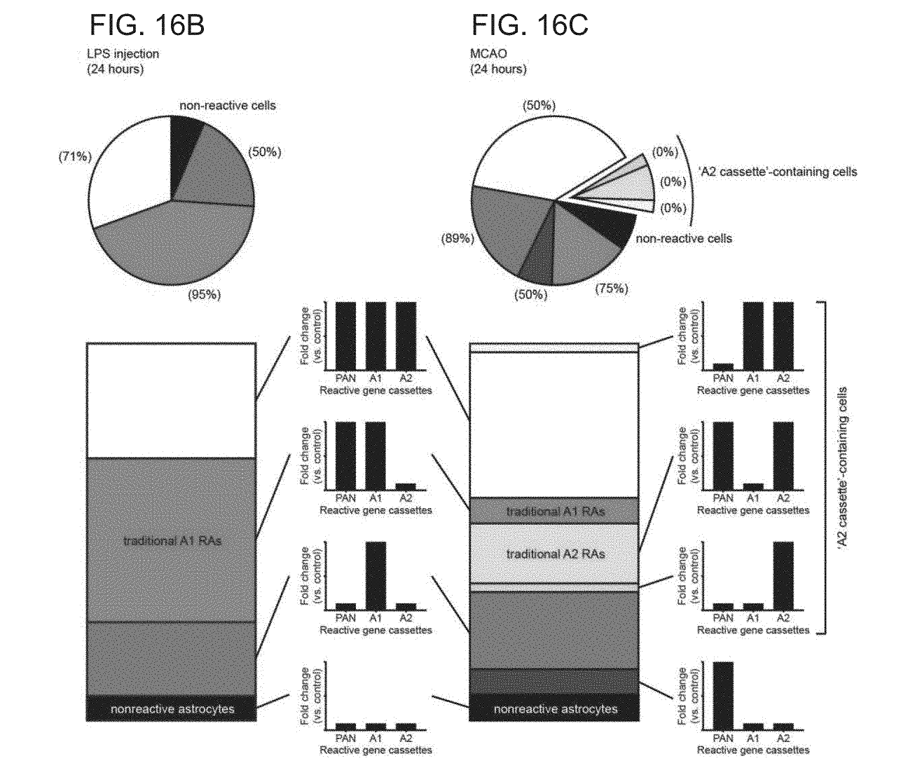

Lastly, because A1s are induced after injury and by LPS (a well-described neurodegeneration sensitizer that causes extensive neuroinflammation), and because reactive microglia are found in neurodegenerative diseases, we investigated whether reactive astrocytes with an A1 phenotype are present in human neuroinflammatory and neurodegenerative diseases. Because complement component C3 is one of the most characteristic and highly upregulated genes in A1s and not expressed by A2 reactive astrocytes (FIG. 16A-16C), we carried out in-situ hybridization and immunochemistry on post-mortem tissue from patients with Alzheimer's disease (AD), Huntington's disease (HD), Parkinson's disease (PD), amyotrophic lateral sclerosis (ALS), and Multiple Sclerosis (MS) to identify whether there is a population of 03-expressing astrocytes in these diseases. In each disease, we found that in regions affected by the disease possess many GFAP and S100 positive astrocytes that are highly 03 immunoreactive (FIG. 5A-5V). In MS in particular, we noted that reactive astrocytes with a hypertrophic morphology and retracted processes have increased C3 immunoreactivity, exhibiting a strong cytoplasmic staining. We were also surprised to see that in demyelinating lesions of MS, C3 positive astrocytes were closely associated with CD68 positive activated microglia/macrophages (FIG. 5F). Similarly, in line with immunoreactivity for C3 protein, C3 mRNA was detected via in situ hybridization specifically in astrocytes (FIGS. 5B-5D) and qPCR analysis reported upregulation of C3 in MS, AD, and PD postmortem tissue samples (FIG. 5A-5V). In human AD, around 40% of GFAP positive astrocytes in the hippocampus were also positive for C3, suggesting that A1 activated astrocytes make up a large proportion of astrocytes in AD, and may be integral for disease initiation and progression. We also report here that the myxovirus (influenza virus) resistance gene MX dynamin Like GTPase 1, MX1, also stains C3 positive A1 reactive astrocytes (FIG. 5A-5V). These findings demonstrate that A1-like reactive astrocytes are present in multiple neuroinflammatory and neurodegenerative diseases acting to drive some aspects of neurodegeneration.

Here we have identified A1 reactive astrocytes, a previously uncharacterized class of reactive astrocytes that are induced by LPS-induced neuroinflammation, acute CNS injury, and acute and chronic neurodegenerative diseases. Our findings demonstrate that A1 reactive astrocytes are induced by classical neuroinflammatory reactive microglia via secretion of II-1α, TNFα, and complement component C1q in vitro and in vivo. Together these three signaling proteins can be used to induce resting astrocytes to an A1 phenotype in vitro in serum-free conditions providing the first culture system of pure A1 reactive astrocytes that very closely resemble their in vivo counterparts. In contrast to A2 reactive astrocytes, which are induced by ischemia⁵ and strongly promote neuronal survival and tissue repair^{10,25-27}, A1s secrete a neurotoxin that induces rapid apoptosis of neurons and mature oligodendrocytes. A1s have lost many characteristic astrocyte functions including the ability to promote neuronal survival and outgrowth, promote synapse formation and function, and to phagocytose synapses and myelin debris. We find that A1s are rapidly induced after acute injury and responsible for the death of axotomized RGCs. A1-like reactive astrocytes are also present in chronic neurodegenerative diseases and their presence may contribute to chronic neuroaxonal damage and drive disease progression.

### Inhibition of IL4α, TNFα and C1q Signaling Prevents A1 Reactive Astrocyte Formation and Increases Neuron Survival

Using marker expression as an assay for the production and activation of A1 astrocytes, the activation of A1 astrocytes was achieved most efficiently in vitro when cells were treated with all three factors, IL-1α, TNFα and C1q (see FIG. 6, “IL-1α+TNFα+C1q”). The marker expression profile obtained by in vitro treatment with IL-1α, TNFα and C1q largely resembled the A1 reactive astrocyte marker profile seen in A1 astrocytes activated in vivo (FIG. 6, compare the expression profile of “IL-1α+TNFα+C1q” to the expression profile of “A1 reactive astrocytes”). In comparison, cells treated with individual factors or pairs of factors, e.g., either IL-1α and TNFα or IL-1α and C1q, produced expression profiles less characteristic of that seen in in vivo produced A1 reactive astrocytes (see FIG. 6, “IL-1α”, “INFα”, “C1q”, “IL-1α+TNFα”, “IL-1α+C1q”).

The blockade of A1 astrocyte activation was investigated in vivo by injecting genetic knockout mice having a functional null mutation in IL-1α, TNFα, C1q or combinations thereof with LPS or saline negative control and assaying astrocyte activation. LPS injected single knockout mice (“IL-1α−/− LPS”, “TNFα−/− LPS”, “C1q−/− LPS” and double knockout mice (“IL-1α−/− TNFα−/− LPS”) showed incomplete blockade of A1 astrocyte activation (FIG. 6). However, when triple knockout mice were injected with LPS complete blockade of A1 astrocyte activation was observed (FIG. 6, “IL-1α−/− TNFα−/− C1q−/− LPS”).

Optic nerve crush was utilized as a representative model of CNS injury in order to assess the relative contributions of IL-1α, TNFα and C1q inhibition to increased RGC survival. Genetic knockout mice having individual functional null mutations in IL-1α, TNFα and C1q as well as double knockout mice (IL-1α and TNFα) and triple knockout mice were used and percent RGC viability was quantified (FIG. 6). As can be seen in the results, only the triple knockout showed 100% RGC viability or better (as compared to non-injured wild type control “WT NO crush”) at the time point assessed. The effect of combined inhibition of IL-1α, TNFα and C1q on survival of RGC neurons following optic nerve crush injury was further investigated using neutralizing antibodies. As a baseline for RGC viability mice having experienced an optic nerve crush were treated with IgG. Individual administration of IL-1α, TNFα and C1q neutralizing antibodies showed modest increases in percent RGC viability following optic nerve crush (see FIG. 6, “IL1α nAb”, “TNFα nAb”, “C1q nAb”). Dual treatment with IL1α and TNFα neutralizing antibodies (“IL1α nAb+TNFα nAb”) showed an increase in percent viability greater than any of the individual administrations and combined administration of ID a, TNFα and C1q neutralizing antibodies (“IL1α nAb+TNFα nAb+C1q nAb”) demonstrated increased RGC survival greater than the sum of the increases seen from each individual neutralizing antibody.

FIG. 1A-1H: A serum-free culture model for A1 reactive astrocytes. FIG. 1A, Heat map of PAN reactive and A1- and A2-specific reactive transcript induction following treatment with a wide range of possible reactivity inducers. Csf1r^{-/-} mice (which lack microglia) fail to produce A1 astrocytes following systemic LPS injection. LPS-activated microglia, or a combination of II-1α, TNFα, and C1q are able to produce A1s in culture. N=8 per group. FIG. 1B, Cytokine array analysis of LPS-activated microglia conditioned media (MCM) shows large increases in II-1α, II-1β and TNFα, however II-1β did not induce A1-specific genes without inducing A2-specific transcripts and as such was not used in future experiments. FIG. 1C, Western blot analysis of LPS-activated MOM with increased C1q protein. FIG. 1D, TGFβ was able to reset A1 reactive astrocytes to a non-reactive state in culture. FIG. 1E, Individual knock-out (II-1α^{-/-}, TNFα^{-/-}, or C1q^{-/-}), double knock-out (II-1α^{-/-}TNFα^{-/-}), and triple knock-out (II-1α^{-/-} TNFα^{-/-}C1q^{-/-}) mice fail to produce A1s following LPS injection. FIG. 1F, Representative phase contrast and fluorescent immunohistochemistry for GFAP and AQP4 micrographs of control and A1 reactive astrocytes (induced by LPS-activated MOM or II-1α, TNFα, and C1q treatment for 24 h). N=8 per group. FIG. 1G, Western blot analysis of GFAP protein levels in cultured astrocytes showing approximate 3-fold increase in A1 reactive astrocytes compared to control. FIG. 1H, Measurements of cross-sectional area of astrocytes stained with GFAP. N=6 for each experiment. * p<0.05, one-way ANOVA. Error bars indicate s.e.m. Scale bar: 50 μm.

FIG. 2A-2G: A1 reactive astrocytes do not promote synapse formation or function. FIG. 2A, Representative images of retinal ganglion cells (RGCs) grown without astrocytes, or with control or A1 reactive astrocytes, immunostained with pre- and post-synaptic markers HOMER (green) and BASSOON (red). Co-localization of these markers (yellow puncta) was counted as a structural synapse. FIG. 2B, Total number of synapses normalized per each individual RGC, n=50 neurons in each treatment. There was a decrease in the number of structural synapses in RGCs treated with A1 astrocyte conditioned media compared to control astrocytes (˜50% decrease). FIG. 2C, Quantitative FOR for astrocyte secreted factors known to be important for synaptogenesis. FIG. 2D, Representative traces of whole-cell patch clamp recordings from RGCs cultured either without or with feeder layers of resting or A1 reactive astrocytes, in the presence of TTX to isolate mEPSCs. Fewer mEPSCs were observed in the presence of A1 reactive astrocytes. FIG. 2E, Frequency of mEPSCs was significantly decreased in the presence of A1 reactive astrocytes (RGCs without astrocytes: 0.19±0.05 Hz n=12 neurons, RGCs with resting astrocytes: 2.28±0.51 Hz n=14 neurons, RGCs with A1 reactive astrocytes: 0.95±0.19 Hz n=16 neurons). FIG. 2F, A1 astrocytes significantly decreased the mean amplitude of mEPSCs (RGCs without astrocytes: 21.81±0.78 pA n=12 neurons, RGCs with resting astrocytes: 23.89±0.38 pA n=14 neurons, RGCs with A1 reactive astrocytes: 22.32±0.37 pA n=16 neurons). FIG. 2G, RGCs cultured with A1 reactive astrocytes had significantly more small amplitude mEPSCs in cumulative probability histograms (p<0.0001 Kolmogorov-Smimov test, n=12-16 neurons per condition). * p<0.05, one-way ANOVA. Error bars indicate s.e.m. Scale bar: 10 μm.

FIG. 3A-3G: A1 astrocytes lose phagocytic capacity. FIG. 3A, Phase and fluorescent images of cultured astrocytes engulfing pHrodo-conjugated synaptosomes (quantification over 24 h in FIG. 3B) and myelin debris (quantification over 12 h in FIG. 3C). A1s phagocytose less synaptosomes and myelin debris compared to control astrocytes. FIG. 3D, Quantitative PCR analysis of astrocyte-specific phagocytic receptors (Megf10 and Mertk, both decrease in A1 reactive astrocytes) and bridging molecules (Gas6 and Axl, unchanged), FIG. 3E, injection protocol for anterograde tracer cholera toxin-β subunit, CTB, into vitreous humor of the eye. FIG. 3F, Representative confocal reconstruction images showing only CTB-labelled retinal ganglion cell (RGC) projections engulfed by control (left) and A1s (right) astrocytes in dorsal lateral geniculate nucleus (dLGN). FIG. 3G, Quantification of engulfment of CTB-labelled RGC projections by astrocytes in the dLGN of control and neuroinflammatory (A1 reactive astrocyte) mice, n=4 per group. A1 reactive astrocytes engulf 50% fewer synapses. * p<0.05, one-way ANOVA, or Student's t-test as appropriate. Error bars indicate s.e.m. Scale bar: 15 μm (FIG. 3A); 10 μm (FIG. 3F).

FIG. 4A-4O: Astrocyte-derived toxic factor promoting cell death. FIG. 4A, Representative phase image showing death of purified retinal ganglion cell (RGC) in culture over 12 h (ethidium homodimer stain in red shows DNA in dead cells). FIG. 4B, FIG. 4C, Quantification of dose-responsive cell death in RGCs (FIG. 4B), and mature differentiated oligodendrocytes (FIG. 4C) caused by A1 reactive astrocyte conditioned media. FIG. 4D, Pan-caspase inhibitor Z-VAD-FMK was able to protect against A1-reactive astrocyte cell mediated death of RGCs. This caspase-inhibition preservation of cell viability was caspase-2 (FIG. 4E) and caspase-3 (FIG. 4F) specific. FIG. 4G, Western blot analysis of cleaved caspase-2 and -3 in RGCs treated with control and A1 reactive astrocyte conditioned media. FIG. 4H, retro-orbital optic nerve crushes (ONC) produced A1s in the retina, while injection of neutralizing antibodies to II-1α, TNFα, and C1q into the vitreous stopped A1 production. FIG. 4I, RBPMS (a marker of RGCs) immunostaining of retinas showed decreased number of RGCs in ONC that was rescued with neutralizing antibody treatment. Quantification of RGC numbers is shown following 7 days (FIG. 4J), 14 days (FIG. 4K) using neutralizing antibodies, and at 7 days using II1α^{-/-} TNFα^{-/-} animals (FIG. 4L) and microglia-depleted animals (FIG. 4M). FIG. 4N, A1 ACM was also found to be toxic to primary human dopaminergic neurons. FIG. 4O, no significant difference was seen in the number of RGCs in ONC mice fed either control chow or PLX-3397 containing chow. * p<0.05, one-way ANOVA. Error bars indicate s.e.m. Scale bar: 100 μm (FIG. 4A); 20 μm (FIG. 4I).

FIG. 5A-5V: A1 reactive astrocytes in human disease. Complement factor 03 is upregulated in A1 reactive astrocytes. FIG. 5A, FIG. 5B, FIG. 5C, Representative in situ hybridization for C3 and immunofluorescent staining for S100β protein to denote astrocytes. Note that in all three pathologies, astrocytes expressing C3 are present. FIG. 5D, co-immunofluorescent staining for C3 and GFAP shows A1 reactive astrocytes in the substantia nigra of human postmortem Parkinsonian brain. Quantification of HD (FIG. 5E), AD (FIG. 5F, FIG. 5G), ALS (FIG. 5H) and PD (FIG. 5I) shows around 40-60% of astrocytes in brain regions specific to each disease in humans are C3 positive, and thus A1 reactive. FIG. 5J, FIG. 5K, FIG. 5L, FIG. 5M, This was coupled with an increase in the expression of C3 transcript in all disease. FIG. 5N, Immunohistochemical staining for C3 shows it is strongly upregulated in astrocytes in active multiple sclerosis (MS) lesions. These astrocytes have a hypertrophic morphology with retracted processes (black arrows). Note hypercellularity indicating extensive infiltration by inflammatory cells in active demyelinating MS lesion of subcortical white matter (cf. luxol fast blue myelin stain of lesion area in right upper corner). FIG. 5O, Immunofluorescent staining showing C3 co-localized with GFAP in cell bodies of reactive astrocytes in acute MS lesions (arrows). Note CD88 positive phagocytes (arrowheads) in proximity to reactive C3 positive astrocytes. See FIG. 5P, FIG. 5Q, and FIG. 5R for single channels and higher magnification of selected area. FIG. 5S, 03 staining pattern in subcortical control white matter is mainly associated with blood vessels and occasionally with resting microglia and fibrous astrocytes. FIG. 5T, in addition to C3, complement factor b (CFB) is also a good marker of A1 s, with 100% colocalisation with GFAP and 03 (data not shown). FIG. 5U, the number of C3+GFAP+ colabelled cells was highest in acute active demyelinating lesions, however they were still present in chronic active and inactive lesions. FIG. 5V, there was a matching increase in C3 transcript in brains of patients with acute active demyelinating lesions compared to age-matched controls. N=3-8 disease and 5-8 control in each instance. Quantification was carried out on 5 fields of view and approximately 50 cells were surveyed per sample. Scale bar: 100 μm (FIG. 5M-5N), 20 μm (FIG. 5O-5S), 10 μm (FIG. 5A-5D). Error bars indicate s.e.m. * p<0.05 , Student's T-test, compared to age-matched control.

### REFERENCES (CITED ABOVE)

1. Sofroniew, M. V. & Vinters, H. V. Astrocytes: biology and pathology. Acta Neuropathol. 119, 7-35 (2010).
2. Clarke, L. E. & Barres, B. A. Emerging roles of astrocytes in neural circuit development. Nat. Rev. Neurosci. 14, 311-21 (2013).
3. Chung, W.-S. S. et al. Astrocytes mediate synapse elimination through MEGF10 and MERTK pathways. Nature 504, 394-400 (2013).
4. Liddelow, S. & Barres, B. SnapShot: Astrocytes in Health and Disease. Cell 162, 1170-1170.el (2015).
5. Zamanian, J. L. et al. Genomic analysis of reactive astrogliosis. J. Neurosci. 32, 6391-410 (2012).
6. Anderson, M. A. et al. Astrocyte scar formation aids central nervous system axon regeneration. Nature 532, 195-200 (2016).
7. Sofroniew, M. V. Astrogliosis. Cold Spring Harb Perspect Biol 7, a020420 (2015).
8. Martinez, F. O. & Gordon, S. The M1 and M2 paradigm of macrophage activation: time for reassessment. F1000Prime Rep 6, 13 (2014).
9. Heppner, F. L., Ransohoff, R. M. & Becher, B. Immune attack: the role of inflammation in Alzheimer disease. Nat. Rev. Neurosci. 16, 358-72 (2015).
10. Bush, T. G. et al. Leukocyte infiltration, neuronal degeneration, and neurite outgrowth after ablation of scar-forming, reactive astrocytes in adult transgenic mice. Neuron 23, 297-308 (1999).
11. Zador, Z., Stiver, S., Wang, V. & Manley, G. T. Role of aquaporin-4 in cerebral edema and stroke. Handb Exp Pharmacol 159-70 (2009). doi:10.1007/978-3-540-79885-9_7
12. Dai, X.-M. M. et al. Targeted disruption of the mouse colony-stimulating factor 1 receptor gene results in osteopetrosis, mononuclear phagocyte deficiency, increased primitive progenitor cell frequencies, and reproductive defects. Blood 99, 111-20 (2002).
13. Cahoy, J. D. et al. A transcriptome database for astrocytes, neurons, and oligodendrocytes: a new resource for understanding brain development and function. J. Neurosci. 28, 264-78 (2008).
14. Zhang, Y. et al. An RNA-sequencing transcriptome and splicing database of glia, neurons, and vascular cells of the cerebral cortex. J. Neurosci. 34, 11929-47 (2014).
15. Zhang, Y. et al. Purification and Characterization of Progenitor and Mature Human Astrocytes Reveals Transcriptional and Functional Differences with Mouse. Neuron 89, 37-53 (2016).
16. Bennett, M. L. et al. New tools for studying microglia in the mouse and human CNS. Proc. Natl. Acad. Sci. U.S.A. 113, E1738-46 (2016).
17. Foo, L. C. et al. Development of a method for the purification and culture of rodent astrocytes. Neuron 71, 799-811 (2011).
18. Kang, W. et al. Astrocyte activation is suppressed in both normal and injured brain by FGF signaling. Proc. Natl. Acad. Sci. U.S.A. 111, E2987-95 (2014).
19. Qiu, G.-P. P. et al. Loss of AQP4 polarized localization with loss of β-dystroglycan immunoreactivity may induce brain edema following intracerebral hemorrhage. Neurosci. Lett. 588, 42-8 (2015).
20. Allen, N. J. et al. Astrocyte glypicans 4 and 6 promote formation of excitatory synapses via GluA1 AMPA receptors. Nature 486, 410-4 (2012).
21. Kucukdereli, H. et al. Control of excitatory CNS synaptogenesis by astrocyte-secreted proteins Hevin and SPARC. Proc. Natl. Acad. Sci. U.S.A. 108, E440-9 (2011).
22. Christopherson, K. S. et al. Thrombospondins are astrocyte-secreted proteins that promote CNS synaptogenesis. Cell 120, 421-33 (2005).
23. Banker, G. A. Trophic interactions between astroglial cells and hippocampal neurons in culture. Science 209, 809-10 (1980).
24. Kigerl, K. A. et al. Identification of two distinct macrophage subsets with divergent effects causing either neurotoxicity or regeneration in the injured mouse spinal cord. J. Neurosci. 29, 13435-44 (2009).
25. Faulkner, J. R. et al. Reactive astrocytes protect tissue and preserve function after spinal cord injury. J. Neurosci. 24, 2143-55 (2004).
26. Okada. S. et al. Conditional ablation of Stat3 or Socs3 discloses a dual role for reactive astrocytes after spinal cord injury. Nat. Med. 12, 829-34 (2006).
27. Herrmann, J. E. et al. STAT3 is a critical regulator of astrogliosis and scar formation after spinal cord injury. J. Neurosci. 28, 7231-43 (2008).
28. Di Giorgio, F. P., Boulting, G. L., Bobrowicz, S. & Eggan, K. C. Human embryonic stem cell-derived motor neurons are sensitive to the toxic effect of glial cells carrying an ALS-causing mutation. Cell Stem Cell 3, 637-48 (2008).
29. Nagai, M. et al. Astrocytes expressing ALS-linked mutated SOD1 release factors selectively toxic to motor neurons. Nat. Neurosci. 10, 615-22 (2007).
30. Reed-Geaghan, E. G., Savage, J. C., Hise, A. G. & Landreth, G. E. CD14 and toll-like receptors 2 and 4 are required for fibrillar A{beta}-stimulated microglial activation. J. Neurosci. 29, 11982-92 (2009).
31. Stevens, B. et al. The classical complement cascade mediates CNS synapse elimination. Cell 131, 1164-78 (2007).
32. Stephan, A. H. et al. A dramatic increase of C1q protein in the CNS during normal aging. J. Neurosci. 33, 13460-74 (2013).
33. Graber, D. J. & Harris, B. T. Purification and culture of spinal motor neurons from rat embryos. Cold Spring Harb Protoc 2013, 319-26 (2013).
34. Zhou, L., Sohet, F. & Daneman, R. Purification of endothelial cells from rodent brain by immunopanning. Cold Spring Harb Protoc 2014, 65-77 (2014).
35. Zhou, L., Sohet, F. & Daneman, R. Purification of pericytes from rodent optic nerve by immunopanning. Cold Spring Harb Protoc 2014, 608-17 (2014).
36. Bustin, S. A. et al. The MIQE guidelines: minimum information for publication of quantitative real-time PCR experiments. Clin. Chem. 55, 611-22 (2009).
37. Winzeler, A. & Wang, J. T. Purification and culture of retinal ganglion cells from rodents. Cold Spring Harb Protoc 2013, 643-52 (2013).
38. Yu, X.-J. J., Liu, M. & Holden, D. W. SsaM and SpiC interact and regulate secretion of Salmonella pathogenicity island 2 type III secretion system effectors and translocators. Mol. Microbial. 54, 604-19 (2004).
39. Zuchero, J. B. et al. CNS myelin wrapping is driven by actin disassembly. Dev. Cell 34, 152-67 (2015).
40. Dunkley, P. R., Jarvie, P. E. & Robinson, P. J. A rapid Percoll gradient procedure for preparation of synaptosomes. Nat Protoc 3, 1718-28 (2008).
41. Larocca, J. N. & Norton, W. T. Isolation of myelin. Curr Protoc Cell Biol Chapter 3, Unit3.25 (2007).
42. Faul, F., Erdfelder, E., Lang, A.-G. G. & Buchner, A. G*Power 3: a flexible statistical power analysis program for the social, behavioral, and biomedical sciences. Behav Res Methods 39, 175-91 (2007).

### REFERENCES (RELATED TO THE BELOW)

Braak H, Del Tredici K, Rüb U, de Vas R A, Jansen Steur E N, Braak E (2003) Staging of brain pathology related to sporadic Parkinson's disease. Neurobiol Aging 24(2):197-211.
Lock C, Hermans G, Pedotti R. Brendolan A, Schadt E, Garren H, Langer-Gould A, Strober S, Cannella B, Allard J, Klonowski P, Austin A, Lad N, Kaminski N, Galli S J, Oksenberg J R, Raine C S, Heller R, Steinman L (2002) Gene-microarray analysis of multiple sclerosis lesions yields new targets validated in autoimmune encephalomyelitis. Nat Med 8(5):500-508.
Frohman E M, Racke M K, Raine C S (2006) Multiple sclerosis-the plaque and its pathogenesis. N Engl J Med 354(9):942-955.
Schirmer L, Srivastava R, Kalluri SR, Böttinger S. Herwerth M, Carassiti D, Srivastava B, Gempt J, Schlegel J, Kuhlmann T, Korn T, Reynolds R, Hemmer B (2014) Differential loss of KIR4.1 immunoreactivity in multiple sclerosis lesions. Ann Neurol 75(6):810-828.

FIG. 7A-7E: FACS analysis of Csf1r^{-/-}mice. FIG. 7A, gating strategy for selecting microglia. Collection of microglia (Tmem119⁺, CD45^{Lo+}Cd11b+) in wild type (Csf1r^{+/+}, FIG. 7B) and knock-out (Csf1r^{-/-}, FIG. 7C) mice. FIG. 7D, there were almost no detectable microglia in Csf1r^{-/-} mice, and no change in wild type or knock-out mice following systemic LPS injection (5 mg/kg). FIG. 7E, the ratio of LPS to saline injected microglia numbers did not change in either wild type or knock-out animals.

FIG. 8A-8C: Screen for A1 reactive mediators. FIG. 8A, Immunopanning schema for purification of astrocytes. These astrocytes retain their non-activated in vivo gene profiles. FIG. 8B, Purified cells were 99+% pure with very little contamination from other central nervous system cells, as measured by qPCR for cell-type specific transcripts. FIG. 8C, Heat map of PAN reactive and A1-and A2-specific reactive transcript induction following treatment with a wide range of possible reactivity inducers. N=8 per experiment. * p<0.05, one-way ANOVA (increase compared to non-reactive astrocytes).

FIG. 9A-9F: Screen for A1 reactive mediators. Fold change data from published microarray datasets of A1 (neuroinflammatory) reactive astrocytes (FIG. 9A), and microfluidic qPCR analysis of purified astrocytes treated with lipopolysaccharide (LPS)-activated microglia conditioned media (FIG. 9B), non-activated microglia conditioned media (FIG. 9C), II-1α, TNFα and C1q (FIG. 9D), LPS-activated microglia conditioned media pre-treated with neutralizing antibodies to II-1α, TNFα and C1q (FIG. 9E), and astrocytes treated with II-1α, TNFα and C1q and post-treated with FGF (FIG. 9F). N=6 per experiment. Error bars indicate s.e.m.

FIG. 10A-10G: A1 astrocytes are morphologically simple. FIG. 10A, FIG. 10B, FIG. 10C, FIG. 100, Quantification of cell morphology of GFAP-stained cultured astrocytes in resting or A1 reactive state: cross-sectional area (FIG. 10A), number of primary processes extending from cell soma (FIG. 10B), number of terminal branchlets (FIG. 10C), ratio of terminal to primary processes (complexity score, FIG. 100). FIG. 10E, FIG. 10F, time-lapse tracing of control (FIG. 10E) and A1 reactive (FIG. 10F) astrocytes. Quantification shown in panel (FIG. 10G). A1 reactive astrocytes migrated approximately 75% less than control astrocytes over a 24 h period. * p<0.05, one-way ANOVA. Error bars indicate s.e.m.

FIG. 11A-11F: A1 reactive astrocytes do not promote synapse formation or neurite outgrowth. FIG. 11A, Representative images of retinal ganglion cells (RGCs) grown without astrocytes, or with control or A1 reactive astrocytes, stained with pre- and post-synaptic markers HOMER (green) and BASSOON (yellow). Colocalization of these markers (yellow puncta) was counted as a structural synapse. FIG. 11B, Total number of synapses normalized per each individual RGC. The number of synapses decreased after growth of RGCs with LPS-activated microglial conditioned media (MCM)-activated A1 reactive astrocyte conditioned media (ACM), or II-1α, TNFα, C1q-activated A1 reactive astrocytes was not different. N=50 neurons in each treatment. FIG. 11C, Quantification of individual pre- and post-synaptic puncta. FIG. 11D, Total length of neurites growth from RGCs. FIG. 11E, Density of RGC processes in cultures used in measurement of synapse number. There was no difference in neurite density close to RGC cell bodies (where synapse number measurements were made). FIG. 11F, Western blot analysis of proteoglycans secreted by control and A1 reactive astrocytes. Conditioned media from control astrocytes contained less chondroitin sulphate proteoglycans Brevican, Ng2, Neurocan and Versican, while simultaneously having higher levels of heparan sulphate proteoglycans Syndecan and Glypican.* p<0.05, one-way ANOVA, except FIG. 11D (Student's t-test). Scale bar: 10 μm. Error bars indicate s.e.m.

FIG. 12: P4 lateral geniculate nucleus astrocytes become A1 reactive following systemic LPS injection. Fold change data from microfluidic qPCR analysis of astrocytes purified from dorsal lateral geniculate nucleus, 24 h after systemic injection with lipopolysaccharide (5 mg/kg). N=2.

FIG. 13A-13R: Astrocyte-derived toxic factor promoting cell death. FIG. 13A, Quantification of dose-responsive cell death in retinal ganglion cells (RGCs) treated with astrocyte conditioned media from cells treated with II-1α, TNFα, or C1q alone, or combination of all three (A1 astrocyte conditioned media, ACM) for 24 h. FIG. 13B, Death of RGCs was not due to a loss of trophic support, as treatment with 50% Control ACM did not decrease viability. Similarly, treatment with a 50/50 mix of Control and A1 ACM did not increase viability compared to A1 ACM only treated cells. FIG. 13C, A1-ACM-induced RGC toxicity could be removed by heat inactivation, or protease treatment. FIG. 13D-13K, Cell viability of purified central nervous system cells treated with A1 ACM for 24 h: RGCs (FIG. 13D), hippocampal neurons (FIG. 13E), embryonic spinal motor neurons (FIG. 13F), oligodendrocyte precursor cells (OPCs, FIG. 13G), astrocytes (FIG. 13F), microglia/macrophages (FIG. 131), endothelial cells (FIG. 13J), and pericytes (FIG. 13K). N=4 for each experiment. FIG. 13L, Representative phase image showing death of purified embryonic spinal motor neurons in culture over 18 h (ethidium homodimer stain in red shows DNA in dead cells). FIG. 13M, qPCR for motor neuronal subtype-specific transcripts after 120h treatment with A1 ACM (50 μg/ml). There was no decrease in levels of transcript for Nr2f2 (pre-ganglionic specific) and Wnt7a and Esrrg (γ specific), suggesting these motor neuron subtypes are immune to A1-induced toxicity. FIG. 13N, representative images with terminal deoxynucleotidyl transferase (TdT) dUTP nick-end labeling (TUNEL) staining in the dentate gyrus for wild type and II1α^{-/-}, TNFα^{-/-}, or C1q^{-/-} individual knockout animals following systemic LPS injection. Individual knock-out animals had far less TUNEL+ cells in the dentate gyrus (no cells in or II1α^{-/-} TNFα^{-/-} animals) than wild type animals—suggesting A1-induced toxicity may be apoptosis. FIG. 13P-13R, Percentage growth rate of gram negative bacterial cultures treated with A1 ACM for 16 h: B. thaliandensis (FIG. 13P), S. typhimurium (FIG. 13Q), S. flexneri (FIG. 13R). N=3. * p<0.05, one-way ANOVA. Error bars indicate s.e.m.

FIG. 14A-14L: Pharmacological blockade of astrocyte derived toxic factor promoting cell death. Specific caspase inhibitory agents tested to block retinal ganglion cell (RGC) cell death: FIG. 14A, caspase-1. FIG. 14B, caspase-4. FIG. 14C, caspase-6. FIG. 14D, caspase-8. FIG. 14E, caspase-9. FIG. 14F, caspase-10. FIG. 14G, caspase-13. Only caspase-4 and caspase-13 inhibition was able to minimize RGC toxicity to A1 ACM (in addition to caspase-2 and -3, see FIG. 4). There was no cleaved caspase-4 or -13 detected in these cells. FIG. 14H, Necrostatin did not preserve RGC viability when cells were treated with A1 astrocyte conditioned media (ACM). FIG. 14I-14L, glutamate excitotoxicity was checked by blocking AMPA receptors with antagonist NBQX (FIG. 14I), or NMDA antagonist D-APS (FIG. 14J), or kainite receptors with antagonist UBP-296 (GluR5 selective, FIG. 14K) and UBP-302 (FIG. 14L)—all of which were ineffective. * p<0.05, one-way ANOVA. N=4 in each. Error bars indicate s.e.m.

FIG. 15A-15K: A1 reactive astrocytes inhibit oligodendrocyte precursor cell differentiation and migration. FIG. 15A, Number of cells counted per day from phase-contrasted images of oligodendrocyte precursor cells (OPCs) treated with control and A1 reactive conditioned media (ACM). FIG. 15B, EdU ClickIt® assay was used to determine percentage growth of OPC cultures treated with increasing concentration of control and A1 reactive ACM for 7 days. Both FIG. 15A and FIG. 15B show that A1 ACM decreases OPC proliferation compared to control ACM-treated OPCs. FIG. 15C, FIG. 150, Representative images of tracked OPC migration following treatment with control (FIG. 15C) and A1 reactive (FIG. 150) ACM. A1 Reactive ACM caused OPCs to travel 55% less distance, as quantified in FIG. 15E. N=100 cells from 10 separate experiments. FIG. 15F, FIG. 15G, 15H, Representative RT-PCR ethidium bromide gel showing no increase in mature OL marker Mbp transcript level in OPCs treated with A1 ACM, with no change in OPC marker Pdgfra and Cspg4 expression—evidence of a lack of differentiation into mature oligodendrocytes. Treatment of OPCs with control ACM did not delay their differentiation into mature oligodendrocytes. N=2. FIG. 15I-15K, Total number of terminal process of oligodendrocyte lineage cells were counted as a measure of differentiation. Over 90% of cells differentiated by 24 h after removal of PDGFα when treated with control ACM (FIG. 15I). In contrast, treatment with a single dose (FIG. 15J) or daily doses (FIG. 15K) of A1 ACM delayed this level of differentiation by 72 h following a single dose, or indefinitely with chronic treatment. N=6 separate experiments. Scale bar: 100 μm. *p<0.05, one-way ANOVA, except he (Student's t-test). Error bars indicate s.e.m.

FIG. 16A-16C: Single cell analysis of C3 expression following neuroinflammatory and ischemic injury. FIG. 16A, cassettes of PAN-, A1-, and A2-specific gene transcripts used to determine polarization state of astrocyte reactivity. Upregulation of combinations of each of these cassettes of genes produces different 8 possible gene profiles for astrocytes following injury. FIG. 16B, 24 hours following LPS-induced systemic neuroinflammation, astrocytes were either non-reactive (no reactive genes upregulated), or fell into three forms of reactivity—all with A1 reactive cassette genes upregulated. Numbers in parenthesis state what percentage of individual cells for each subtype were expressing C3. FIG. 16C, 24 hours following MCAO ischemia, both neuroinflammatory (A1 and A1-like) and ischemic (A2 and A2-like) reactive cells were detected. No cells expressing A2 cassette transcripts were C3 positive—validating C3 as an appropriate marker for visualizing A1 reactive astrocytes in disease.

TABLE 2
Rat primer sequences.

| | | SEQ ID | | SEQ ID | PRODUCT |
|---|---|---|---|---|---|
| ID | FWD | NO. | REV | NO. | SIZE |
| Aif1 | AAGGATTTGCAGGGAGGAAAAGC | 7 | CTCCATGTACTTCGTCTTGAAGG | 70 | 156 |
| Aldh1/1 | AGTGAAGGAGCTGTGTGACG | 8 | TCCATCCGTTGGGTTGATGG | 71 | 253 |
| Amigo2 | GTTCGCCACAACAACATCAC | 9 | GTTTCTGCAAGTGGGAGAGC | 72 | 211 |
| Aqp4 | AACCCCAGAAGACAGCACCT | 10 | ACACTTACAGCTGTCCAGGGTTG | 73 | 76 |
| Aspg | CAGGTGCCCAGGTTCCTATC | 11 | GTCCACCTTGGTTGTCCGAT | 74 | 152 |
| Axl | GACACCCCCGAGGTACTTATG | 12 | TGGGGGTTCACTCACTGGG | 75 | 177 |
| B3gnt5 | TGCTCCTGGATGAAAGGTCC | 13 | ACATGCTTGATCCGTGTGGT | 76 | 161 |
| Cd109 | GTCGCTCACAGGTACCTCAA | 14 | CTGTGAAGTTGAGCGTTGGC | 77 | 116 |
| Cd14 | TCAGAATCTACCGACCATGAAGC | 15 | GGACACTTTCCTCGTCCTGG | 78 | 119 |
| Cd44 | TCAGGATAGCCCCACAACAAC | 16 | GACTCCGTACCAGGCATCTTC | 79 | 159 |
| Cd68 | CGCATCTTGTACCTGACCCA | 17 | TTCTGCGCTGAGAATGTCCA | 80 | 254 |
| Clcf1 | GACTCGTGGGGGATGTTAGC | 18 | CCCCAGGTAGTTCAGGTAGGT | 81 | 180 |
| Cp | GATGTTTCCCCAAACGCCTG | 19 | GTAGCTCTGAGACGATGCTTGA | 82 | 118 |
| Cx3cr1 | TTCCTGCAGAAGTCCCCGT | 20 | CCGAACGTGAAGACAAGGGA | 83 | 179 |
| Cxcl10 | TGCAAGTCTATCCTGTCCGC | 21 | ACGGAGCTCTTTTTGACCTTC | 84 | 140 |
| Emp1 | ACCATTGCCAACGTCTGGAT | 22 | TGGAACACGAAGACCACGAG | 85 | 188 |
| Fbln5 | AGGGGGTTAAGCGAAACCAG | 23 | GTGAGTATCCTTTTAATCCTGGCA | 86 | 198 |
| Fkbp5 | TGCAGTGTCGGCAGTTGTAT | 24 | GGGTCGCCCAAGTTAGAACA | 87 | 112 |
| Gabra1 | TCCATGATGGCTCAAACCGT | 25 | TCTTCATCACGGGCTTGTCC | 88 | 183 |
| Gapdh | GTGCCAGCCTCGTCTCATAG | 26 | AGAGAAGGCAGCCGTGGTAA | 89 | 91 |
| Gas6 | ACCTCGTCCAGAAGATTAAC | 27 | TCCGGGTGTAGTTGAGGCTA | 90 | 189 |
| Gbp2 | TAAAGGTCCGAGGCCCAAAC | 28 | AACATATGTGGCTGGGCGAA | 91 | 192 |
| Gfap | AACCGCATCACCATTCCTGT | 29 | TCCTTAATGACCTCGCCATCC | 92 | 146 |
| Ggta1 | TCTCAGGATCTGGGAGTTGGA | 30 | GAGTTCTATGGAGCTCCCGC | 93 | 84 |
| Gjc2 | GGAAGGGCTCATCAGAAGGT | 31 | CCGTTAGCACAATGCGGAAG | 94 | 179 |
| Gpc4 | TGGACCGACTGGTTACTGATG | 32 | CCCTGGTTGGCTAATCCGTT | 95 | 190 |
| Gpc6 | TTTCGACCCTACAACCCGGA | 33 | GTCTGTGACACTGTGCTGCAT | 96 | 102 |
| H2-D1 | ATGGAACCTTCCAGAAGTGGG | 34 | GAAGTAAGTTGGAGTCGGTGGA | 97 | 144 |
| H2-T23 | ATTGGAGCTGTTGTGAGGAGG | 35 | CCACGAGGCAACTGTCTTTTC | 98 | 130 |
| Hsbp1 | GAGATCACTGGCAAGCACGA | 36 | ATTGTGTGACTGCTTTGGGC | 99 | 172 |
| ligp1 | ATTTGGCTCGAAGCCTTTGC | 37 | ACGGCATTTGCCAGTCCTTA | 100 | 169 |
| Itgam | GACTCCGCATTTGCCCTACT | 38 | TGCCCACAATGAGTGGTACAG | 101 | 109 |
| Lcn2 | CCGACACTGACTACGACCAG | 39 | AATGCATTGGTCGGTGGGAA | 102 | 197 |
| Mbp | AGGCGTAGAGGAACTATGGGT | 40 | TCACCACTGTCCAATCAGGG | 103 | 125 |
| Megf10 | TACCGCCATGGGGAGAAAAC | 41 | TTATCAGCGCAGTGAGGGAC | 104 | 98 |
| Mertk | CTGCTTCTGCGGGTTTGTTC | 42 | GGCTTTGCAAGGTAAGCTCG | 105 | 179 |
| Mog | AACTCCGTGCAGAAGTCGAG | 43 | TCACTCAAAAGGGGTTTCTTAGC | 106 | 195 |
| Nefl | AAGCACGAAGAGCGAGATGG | 44 | ACCTGCGAGCTCTGAGAGTA | 107 | 177 |
| Osmr | GTCATTCTGGACATGAAGAGGT | 45 | AATCACAGCGTTGGGTCTGA | 108 | 144 |
| Psmb8 | TATCTGCGGAATGGGGAACG | 46 | AAAGTCCCGGTCCCTTCTTG | 109 | 136 |
| Ptgs2 | CTCAGCCATGCAGCAAATCC | 47 | GGGTGGGCTTCAGCAGTAAT | 110 | 172 |
| Ptx3 | CATCCCGTTCAGGCTTTGGA | 48 | CACAGGGAAAGAAGCGAGGT | 111 | 104 |
| Rplp0 | CCCACTGGCTGAAAAGGTCA | 49 | TTGGTGTGAGGGGCTTAGTC | 112 | 192 |
| S100a10 | GAAAGGGAGTTCCCTGGGTT | 50 | CCCACTTTTCCATCTCGGCA | 113 | 98 |
| S1pr3 | CTTGCAGAACGAGAGCCTGT | 51 | CCTCAACAGTCCACGAGAGG | 114 | 70 |
| Serpina3n | GTCTTTCAGGTGGTCCACAAGG | 52 | GCCAATCACAGCATAGAAGCG | 115 | 297 |
| Serping1 | TGGCTCAGAGGCTAACTGGC | 53 | GAATCTGAGAAGGCTCTATCCCCA | 116 | 122 |
| Slc10a6 | TCCATAGAGACCGGAGCACA | 54 | ATGCCTGATATGCTGCGACA | 117 | 157 |
| Snap25 | GGATGAGCAAGGCGAACAAC | 55 | TCCTGATTATTGCCCCAGGC | 118 | 180 |
| Sox10 | GACCCTATTATGGCCACGCA | 56 | GCCCCTCTAAGGTCGGGATA | 119 | 182 |
| Sparc | AAAACGTCCTGGTCACCTTG | 57 | TGGGACAGGTACCCATCAAT | 120 | 232 |
| Sparcl1 | CAGTCCCGACAACGTTTCTCT | 58 | CTGTCGACTGTTCATGGGCT | 121 | 186 |
| Sphk1 | AAAGCGAGACCCTGTTCCAG | 59 | CAGTCTGCTGGTTGCATAGC | 122 | 231 |
| Srgn | GTTCAAGGTTATCCTGCTCGGA | 60 | AAACAGGATCGGTCATCGGG | 123 | 151 |
| Steap4 | CAAACGCCGAGTACCTTGCT | 61 | CAGACAAACACCTGCCGACT | 124 | 121 |
| Syt1 | AGCCATAGTTGCGGTCCTTT | 62 | TCAGTCAGTCCGGTTTCAGC | 125 | 189 |
| Tgm1 | AGACCCAATTTTCCTGGGGC | 63 | AGCGAGGACCTTCCATTGTG | 126 | 100 |
| Thbs1 | TCGGGGCAGGAAGACTATGA | 64 | ACTGGGCAGGGTTGTAATGG | 127 | 118 |
| Thbs2 | CGTGAGCGATGAGAAGGAGA | 65 | CGATCTGTGCTTGGTTGTGC | 128 | 122 |
| Timp1 | CGCTAGAGCAGATACCACGA | 66 | CCAGGTCCGAGTTGCAGAAA | 129 | 140 |
| Tm4sf1 | CTGAGGGACAGTACCTTCTGGATTC | 67 | GGCTAGGCCTCAACACAGTTA | 130 | 225 |
| Ugt1a | GGAAGCTGTTAGTGATCCCC | 68 | TGCTATGACCACCACTTCGT | 131 | 101 |
| Vim | GAGGAGATGAGGGAGTTGCG | 69 | CTGCAATTTTTCTCGCAGCC | 132 | 117 |

TABLE 3
Mouse primer sequences.

| | | SEQ ID | | SEQ ID | PRODUCT |
|---|---|---|---|---|---|
| ID | FWD | NO. | REV | NO. | SIZE |
| Aif1 | GGATCAACAAGCAATTCCTCGA | 133 | CTGAGAAAGTCAGAGTAGCTGA | 195 | 247 |
| Aldh1/1 | GCAGGTACTTCTGGGTTGCT | 134 | GGAAGGCACCCAAGGTCAAA | 196 | 86 |
| Amigo2 | GAGGCGACCATAATGTCGTT | 135 | GCATCCAACAGTCCGATTCT | 197 | 263 |
| Aqp4 | CTGGGCATCCTGTCACAACA | 136 | CAGGAATGTCCACACTTAGACAC | 198 | 94 |
| Arg1 | TTTTAGGGTTACGGCCGCGGTG | 137 | CCTCGAGGCTGTCCTTTTGA | 199 | 146 |
| Aspg | GCTGCTGGCCATTTACACTG | 138 | GTGGGCCTGTGCATACTCTT | 200 | 133 |
| B3gnt5 | CGTGGGGCAATGAGAACTAT | 139 | CCCAGCTGAACTGAAGAAGG | 201 | 207 |
| Ccl2 | CACTCACCTGCTGCTACTCA | 140 | GCTTGGTGACAAAAACTACAGC | 202 | 117 |
| Cd109 | CACAGTCGGGAGCCCTAAAG | 141 | GCAGCGATTTCGATGTCCAC | 203 | 147 |
| Cd14 | GGACTGATCTCAGCCCTCTG | 142 | GCTTCAGCCCAGTGAAAGAC | 204 | 232 |
| Cd44 | ACCTTGGCCACCACTCCTAA | 143 | GCAGTAGGCTGAAGGGTTGT | 205 | 299 |
| Cd68 | ACTGGTGTAGCCTAGCTGGT | 144 | CCTTGGGCTATAAGCGGTCC | 206 | 85 |
| Celf4 | TGCGCTTTCCTCACCTACTG | 145 | TTTCTATGTGAAGGGGGCTGG | 207 | 111 |
| Clcf1 | CTTCAATCCTCCTCGACTGG | 146 | TACGTCGGAGTTCAGCTGTG | 208 | 176 |
| Cp | TGTGATGGGAATGGGCAATGA | 147 | AGTGTATAGAGGATGTTCCAGGTCA | 209 | 282 |
| Cx3cr1 | CAGCATCGACCGGTACCTT | 148 | GCTGCACTGTCCGGTTGTT | 210 | 65 |
| Cxcl10 | CCCACGTGTTGAGATCATTG | 149 | CACTGGGTAAAGGGGAGTGA | 211 | 211 |
| Emp1 | GAGACACTGGCCAGAAAAGC | 150 | TAAAAGGCAAGGGAATGCAC | 212 | 183 |
| Fbln5 | CTTCAGATGCAAGCAACAA | 151 | AGGCAGTGTCAGAGGCCTTA | 213 | 281 |
| Fkbp5 | TATGCTTATGGCTCGGCTGG | 152 | CAGCCTTCCAGGTGGACTTT | 214 | 194 |
| Gabra1 | GCTTCCTAGCTTGCGTTCATT | 153 | AACTTGCACTCTGGCCCTAA | 215 | 293 |
| Gapdh | AAGAGGGATGCTGCCCTTAC | 154 | TACGGCCAAATCCGTTCACA | 216 | 119 |
| Gbp2 | GGGGTCACTGTCTGACCACT | 155 | GGGAAACCTGGGATGAGATT | 217 | 285 |
| Gfap | AGAAAGGTTGAATCGCTGGA | 156 | CGGCGATAGTCGTTAGCTTC | 218 | 299 |
| Gfap | AGAAAGGTTGAATCGCTGGA | 157 | CGGCGATAGTCGTTAGCTTC | 219 | 299 |
| Ggta1 | GTTGAACAGCATGAGGGGTTT | 158 | GTTTTGTTGCCTCTGGGTGT | 220 | 115 |
| Gjc2 | CTTGTGCATCTCCAGGTCCCA | 159 | TGTCAGCACAATGCGGAAGA | 221 | 151 |
| H2-D1 | TCCGAGATTGTAAAGCGTGAAGA | 160 | ACAGGGCAGTGCAGGGATAG | 222 | 204 |
| H2-T23 | GGACCGCGAATGACATAGC | 161 | GCACCTCAGGGTGACTTCAT | 223 | 212 |
| Hsbp1 | GACATGAGCAGTCGGATTGA | 162 | GGATGGGGTGTAGGGGTACT | 224 | 265 |
| ligp1 | GGGGCAATAGCTCATTGGTA | 163 | ACCTCGAAGACATCCCCTTT | 225 | 104 |
| ll1a | CGCTTGAGTCGGCAAAGAAAT | 164 | CTTCCCGTTGCTTGACGTTG | 226 | 271 |
| ll1b | TGCCACCTTTTGACAGTGATG | 165 | TGATGTGCTGCTGCGAGATT | 227 | 138 |
| Itgam | TGGCCTATACAAGCTTGGCTTT | 166 | AAAGGCCGTTACTGAGGTGG | 228 | 93 |
| Lcn2 | CCAGTTCGCCATGGTATTTT | 167 | CACACTCACCACCCATTCAG | 229 | 206 |
| Marco | TTCTGTCGCATGCTCGGTTA | 168 | CAGATGTTCCCAGAGCCACC | 230 | 71 |
| Mbp | GAGACCCTCACAGCGATCCAAG | 169 | GGAGGTGGTGTTCGAGGTGTC | 231 | 282 |
| Mog | CACCGAAGACTGGCAGGACA | 170 | CCACAGCAAAGAGGCCAATG | 232 | 129 |
| Msr1 | CCAGCAATGACAAAAGAGATGACA | 171 | CTGAAGGGAGGGGCCATTTT | 233 | 150 |
| Nefl | CAAGGACGAGGTGTCGGAAA | 172 | TGATTGTGTCCTGCATGGCG | 234 | 152 |
| Osmr | GTGAAGGACCCAAAGCATGT | 173 | GCCTAATACCTGGTGCGTGT | 235 | 199 |
| Psmb8 | CAGTCCTGAAGAGGCCTACG | 174 | CACTTTCACCCAACCGTCTT | 236 | 121 |
| Ptgs2 | GCTGTACAAGCAGTGGCAAA | 175 | CCCCAAAGATAGCATCTGGA | 237 | 232 |
| Ptx3 | AACAAGCTCTGTTGCCCATT | 176 | TCCCAAATGGAACATTGGAT | 238 | 147 |
| S100a10 | CCTCTGGCTGTGGACAAAAT | 177 | CTGCTCACAAGAAGCAGTGG | 239 | 238 |
| S1pr3 | AAGCCTAGCGGGAGAGAAAC | 178 | TCAGGGAACAATTGGGAGAG | 240 | 197 |
| Saa3 | GGGTCTAGAGACATGTGGCG | 179 | TCTGGCATCGCTGATGACTT | 241 | 150 |
| Serpina3n | CCTGGAGGATGTCCTTTCAA | 180 | TTATCAGGAAAGGCCGATTG | 242 | 233 |
| Serping1 | ACAGCCCCCTCTGAATTCTT | 181 | GGATGCTCTCCAAGTTGCTC | 243 | 299 |
| Slc10a6 | GCTTCGGTGGTATGATGCTT | 182 | CCACAGGCTTTTCTGGTGAT | 244 | 217 |
| Snap25 | AGCAAGGCGAACAACTCGAT | 183 | AGGCCACAGCATTTGCCTAA | 245 | 106 |
| Sphk1 | GATGCATGAGGTGGTGAATG | 184 | TGCTCGTACCCAGCATAGTG | 246 | 135 |
| Srgn | GCAAGGTTATCCTGCTCGGA | 185 | TGGGAGGGCCGATGTTATTG | 247 | 134 |
| Steap4 | CCCGAATCGTGTCTTTCCTA | 186 | GGCCTGAGTAATGGTTGCAT | 248 | 262 |
| Syt1 | CGCTCCAGTTTCCCTCTGAAT | 187 | GGATGTTGGTTGTTCGAGCG | 249 | 126 |
| Tgm1 | CTGTTGGTCCCGTCCCAAA | 188 | GGACCTTCCATTGTGCCTGG | 250 | 97 |
| Timp1 | AGTGATTTCCCCGCCAACTC | 189 | GGGGCCATCATGGTATCTGC | 251 | 123 |
| Tm4sf1 | GCCCAAGCATATTGTGGAGT | 190 | AGGGTAGGATGTGGCACAAG | 252 | 258 |
| Tmem119 | GTGTCTAACAGGCCCCAGAA | 191 | AGCCACGTGGTATCAAGGAG | 253 | 119 |
| Tnfa | TGTGCTCAGAGCTTTCAACAA | 192 | CTTGATGGTGGTGCATGAGA | 254 | 88 |
| Ugt1a | CCTATGGGTCACTTGCCACT | 193 | AAAACCATGTTGGGCATGAT | 255 | 136 |
| Vim | AGACCAGAGATGGACAGGTGA | 194 | TTGCGCTCCTGAAAAACTGC | 256 | 169 |

TABLE 4
Human postmortem tissue samples from Parkinson's disease patients, and age matched controls.

| | Age | | PMD | | | | Brain |
|---|---|---|---|---|---|---|---|
| Sex | (years) | Race | (hours) | FDX | CERAD | BRAAK | region |
| M | 76 | W | 18 | PD | 0 | 2 | SN |
| M | 86 | W | 19 | Lewy body disease, incipient AD | 0 | 2 | SN |
| M | 90 | W | 7 | PD, neurofibrillary tangles and tau | 0 | 4 | SN |
| | | | | pathology BRAAK 4, TBI possible | | | |
| M | 92 | W | 17 | PD, dementia | 0 | 3 | SN |
| M | 80 | W | 9.5 | PD, dementia | 0 | 3 | SN |
| F | 85 | W | 19 | PD, dementia, FTD, cerebrovascular disease | 0 | 4 | SN |
| M | 76 | W | 13.5 | PD | 0 | 1 | SN |
| M | 76 | W | 25 | Control | NA | NA | SN |
| M | 82 | W | 20 | Control | NA | NA | SN |
| M | 81 | W | 26 | Control | NA | NA | SN |
| M | 76 | W | 9 | Control | NA | NA | SN |
| M | 83 | W | 25 | Control, vascular disease | NA | NA | SN |

Abbreviations: AD, Alzheimer's disease; BRAAK, Braak staging (Braak et al., 2003); CERAD, Consortium to Establish a Registry for Alzheimer's Disease (CERAD) neurocognitive test battery result; F, female; FDX, functional diagnosis; FTD, Frontotemporal dementia; M, male; PMD, post mortem delay; SN, substantia nigra; NA, not applicable; TBI, traumatic brain injury; W, white (Caucasian).

TABLE 5
Clinical and pathological characteristics of multiple sclerosis
patients, and age-matched controls

| | | | Disease | | |
|---|---|---|---|---|---|
| | Age | PMD | duration | Disease | |
| Sex | (years) | (hours) | (years) | course | FDX |
| F | 51 | 10 | 23 | SP | active |
| F | 35 | 9 | 5 | SP | active |
| M | 40 | 27 | 16 | SP | active |
| F | 50 | 22 | 23 | SP | active, chronic inactive |
| F | 42 | 11 | 6 | PP | chronic active |
| F | 34 | 12 | 11 | SP | chronic active |
| F | 59 | 21 | 39 | SP | chronic active |
| F | 59 | 21 | 39 | SP | chronic active |
| F | 53 | 17 | 28 | SP | chronic inactive |
| M | 53 | 13 | 16 | SP | chronic inactive |
| F | 57 | 12 | 19 | SP | chronic inactive |
| M | 82 | 21 | NA | NA | control, unknown |
| M | 35 | 22 | NA | NA | control, carcinoma of the |
| | | | | | tongue |
| M | 84 | 5 | NA | NA | control, carcinoma of the |
| | | | | | bladder |
| M | 82 | 21 | NA | NA | control, myelodysplastic |
| | | | | | syndrome |

Inflammatory staging of subcortical MS lesions was carried out according to established histological criteria: active - presence of MOG+/LFB+ phagocytes and strong microglia activation; early inactive - presence of PAS+ phagocytes and strong microglia activation; late inactive - no macrophages and diffuse microglia activation (Lock et al., 2002; Frohman et al., 2006; Schirmer et al., 2014).
Abbreviations:
F, female;
FDX, functional diagnosis;
LFB, Luxol fast blue;
M, male;
MOG, myelin oligodendrocyte glycoprotein;
MS, multiple sclerosis;
NA, not applicable;
PAS, periodic acid Schiff;
PP, primary progressive MS;
SP, secondary progressive MS.

TABLE 6
Clinical and pathological characteristics of Alzheimer's
disease patients, and age-matched controls.

| | Age | PMD | | |
|---|---|---|---|---|
| Sex | (years) | (hours) | FDX | Brain region |
| M | 89 | 8.75 | AD | PFC |
| F | 80 | 7 | AD | PFC |
| F | 79 | 9.5 | AD | RFC |
| M | 79 | — | control, unknown | PFC |
| M | 80 | — | control, unknown | PFC |
| F | 82 | — | control, unknown | PFC |
| M | 81 | — | control, unknown | PFC |
| M | 84 | — | control, unknown | RFC |
| F | 90 | — | control, unknown | PFC |
| F | 61 | 6 | AD | Hippocampus |
| F | 85 | 14 | AD | Hippocampus |
| F | 76 | 23 | AD | Hippocampus |
| F | 56 | 12 | control, unknown | Hippocampus |
| — | — | — | control, unknown | Hippocampus |
| — | — | — | control, unknown | Hippocampus |

TABLE 7
Clinical and pathological characteristics of amyloid
lateral sclerosis patients, and age-matched controls.

| | Age | PMD | | Brain | | |
|---|---|---|---|---|---|---|
| Sex | (years) | (hours) | FDX | Atrophy | Dementia | Brain region |
| F | 67 | 19 | ALS | None | No | Motor cortex |
| M | 67 | 8 | ALS | None | No | Motor cortex |
| M | 56 | 4 | ALS | Severe | No | Motor cortex |
| F | 56 | 12 | Control | None | No | Motor cortex |
| — | — | — | Control | None | No | Motor cortex |
| — | — | — | Control | None | No | Motor cortex |

TABLE 8
Clinical and pathological characteristics of Huntington's
disease patients, and age-matched controls.

| | Age | PMD | | CAG | Vonsattel | |
|---|---|---|---|---|---|---|
| Sex | (years) | (hours) | FDX | Number | grade | Brain region |
| F | 59 | 7 | HD | 47 | HD4 | Caudate nucleus |
| M | 54 | 8 | HD | 46 | HD4 | Caudate nucleus |
| F | 45 | 16 | HD | Unknown | HD4 | Caudate nucleus |
| M | 51 | 16 | Control | Unknown | N/A | Caudate nucleus |
| M | 54 | 6.5 | Control | Unknown | N/A | Caudate nucleus |
| F | 63 | 16 | Control | 16 | N/A | Caudate nucleus |
| M | 60 | 17 | Control | 17 | N/A | Caudate nucleus |
| M | 41 | 16 | Control | 22 | N/A | Caudate nucleus |

### Example 2: Blocking Formation of A1 Reactive Astrocytes Preserves Neuronal Health in a Mouse Model of Glaucoma

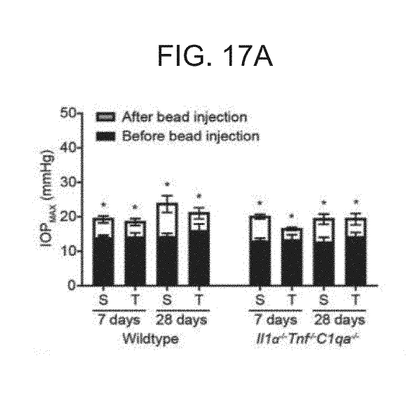

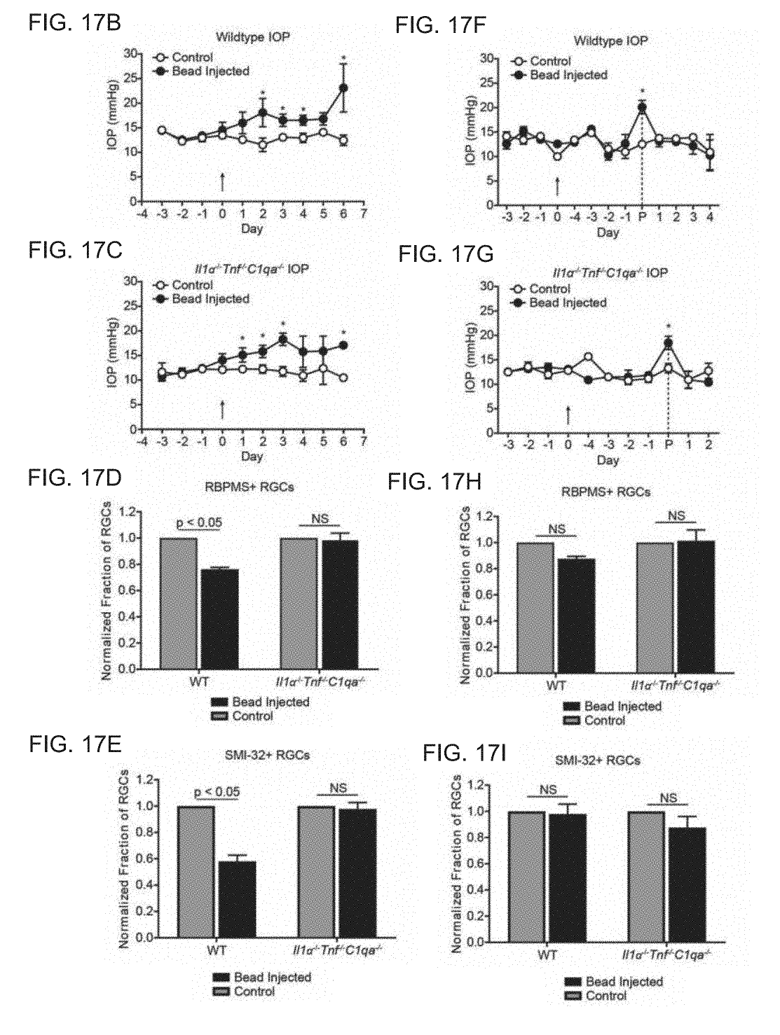

A sustained and moderate elevation of intraocular pressure provides a useful model for research into the mechanisms of glaucomatous retinal damage. In the instant example, a bead injection-induced model of glaucoma was used in mice to investigate the role of A1 astrocytes in glaucoma mediated RGC death. Maximum intraocular pressure (10P) measurements were performed before and after bead injection in wildtype and IL II1α−/−Tnf−/−C1qa−/− (A1-deficient) mice following transient (T) or sustained (S) pressure increase (FIG. 17A). In both groups a measureable increase in IOP was observed following bead injection, but no statistical difference was seen between groups (p<0.05, one-way ANOVA). Daily average measurements of IOP in control and bead injected wildtype and II1α−/−Tnf−/−C1qa−/− mice show a sustained 10P increase in bead injected animals of both groups (FIG. 17B-17C).

Staining for RNA-binding protein with multiple splicing (RBPMS), a marker for all RGCs in the mouse retina, was used to quantify the number of RGCs present 1 month following sustained 10P increase in the retinas of control and bead injected wildtype or II1α−/−Tnf−/−C1qa−/− mice. The resulting quantification is provided in FIG. 17D. As can be seen in the data, wildtype animals showed a ˜20% decrease in the number of RGCs present in bead injected retinas as compared to non-injected controls. However, the numbers of RGCs present in injected and non-injected control II1α−/−Tnf−/−C1qa−/− mice were the same. These data demonstrate a pan-RGC decrease in viability following sustained 10P increase in wildtype mice that does not occur in the A1 deficient II1α−/−Tnf−/−C1qa−/− mice subjected to the same sustained 10P increase. This finding was further confirmed in a subpopulation of RGCs stained with SMI-32 (monoclonal antibody to non-phosphorylated neurofilaments) which demonstrated an even greater decrease in viability (˜40%) within the RGC subpopulation in the wildtype mice and no decrease in viability in the II1α−/−Tnf−/−C1qa−/− mice.

To assess the specificity of this response to sustained 10P increase, the assays were also performed in the context of transient 10P increase. As can be seen in the provided data, daily measurements demonstrated comparable transient 10P increases, in the bead injected eyes as compared to non-injected control eyes, between wildtype and II1α−/−Tnf−/−C1qa−/− mice in (FIG. 17F-17G). However, no change in the numbers of RBPMS positive (FIG. 17H) or SMI-32 positive (FIG. 17I) RGCs in response to transient 10P increase were observed in either wildtype or II1α−/−Tnf−/−C1qa−/− mice.

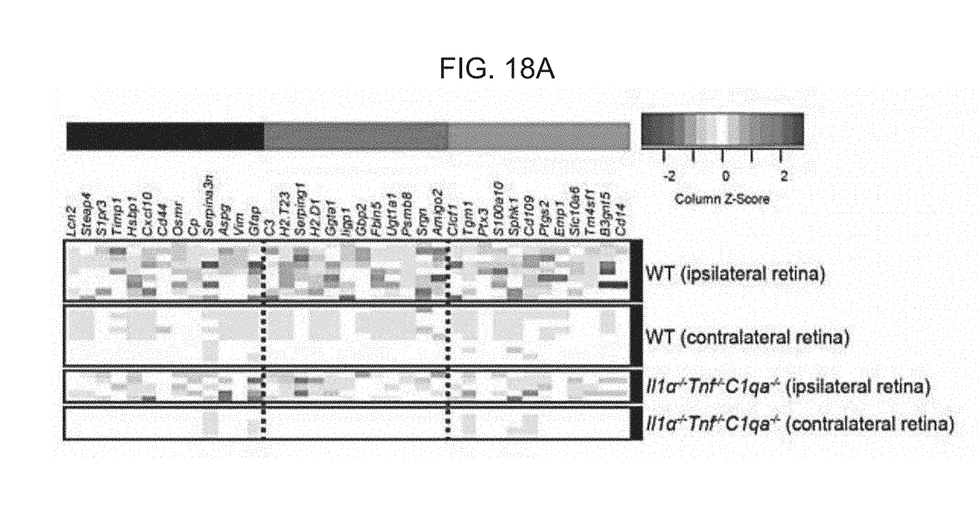

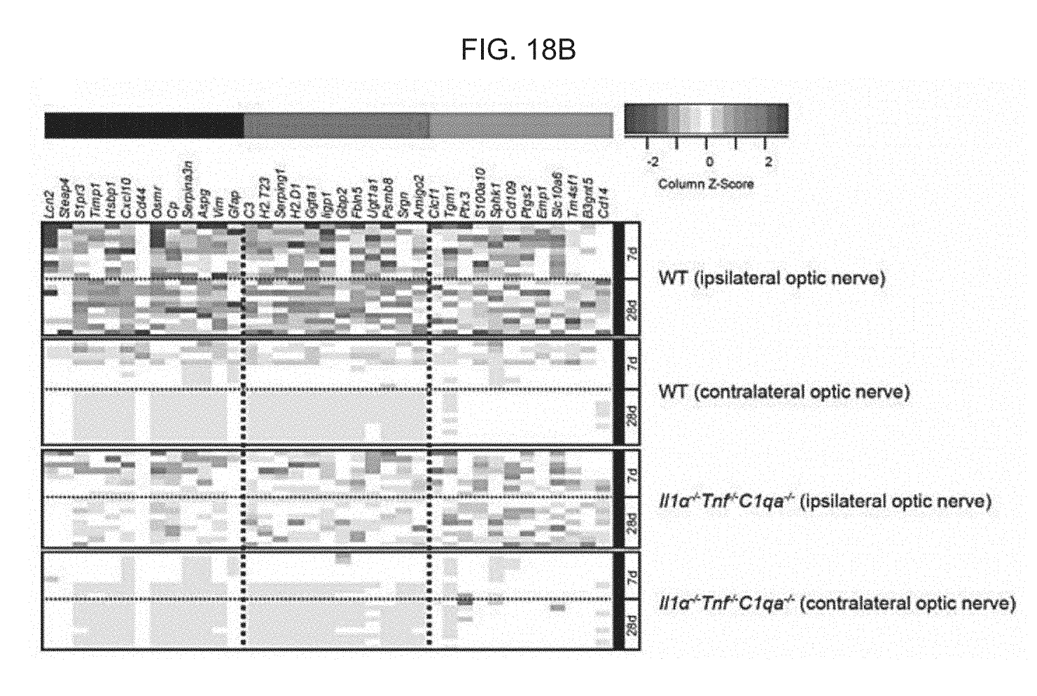

Gene expression analysis in the mouse model of glaucoma was also performed. Heatmap analysis of reactive astrocyte transcripts in the retina 7 days following bead injection is provided in FIG. 18A. Wildtype (WT) injected (ipsilateral) retinas had upregulation of multiple astrocyte reactive transcripts, while contralateral (uninjected) eyes did not. There was essentially no change in reactive gene expression in II1α−/−Tnf−/−C1qa−/− animals in either eye. For comparison, heatmap analysis of reactive astrocyte transcripts in the optic nerve head 7 and 28 days following bead injection is also provided (FIG. 18B). WT (ipsilateral) retinas had upregulation of multiple astrocyte reactive transcripts at both 7 and 28 days, while contralateral (uninjected) eyes did not. II1α−/−Tnf−/−C1qa−/− animals had minimal change in expression of reactive transcripts, with essentially no upregulation of PAN and A1-specific transcripts.

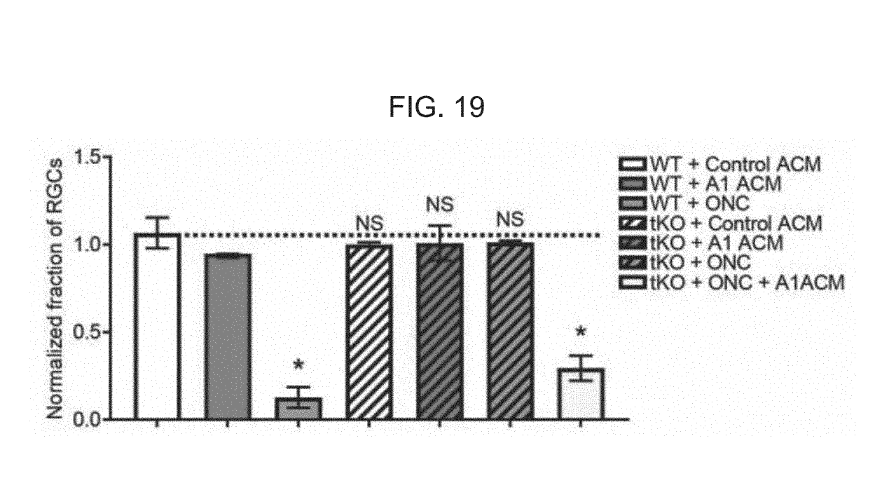

To further assess the role of A1 astrocyte formation in retinal cell viability, RGC viability was assessed by RBPMS staining of whole mount retinas following optic nerve crush in wildtype (WT) and II1α−/−Tnf−/−C1qa−/− (tKO) animals and quantification of RGC survival was performed (FIG. 19). Animals received an intraocular injection of either non-toxic control astrocyte conditioned media (ACM), toxic ACM (A1 ACM), or no injection. Injections were paired with or without a retroorbital optic nerve crush (ONC). Only RGCs in animals with damaged neurons (WT+ONC, or tKO+ONC+A1 ACM) died. Whereas, crush alone in the absence of astrocyte toxin (tKO+ONC), or absence of nerve injury but presence of toxin (WT+A1 ACM, tKO+ONC) did not cause death of RGCs. Accordingly, in conjunction with the above described data showing a lack of RGC death in response to transient 10P increase which may not substantially damage neurons, a combination of neuronal injury and the presence of A1 astrocyte-derived toxicity is necessary for induction of RGC death in these models.

Collectively, the data of the instant example show that A1 reactive astrocytes are induced in a mouse model of glaucoma and that activation of such cells correlates with RGC neuronal death. Furthermore, a lack of RGC death in the II1α−/−Tnf−/−C1qa−/− mice demonstrates that blocking formation of A1 astrocytes can preserve neuronal health in general and maintain RGC viability in glaucoma specifically.

### Example 3: Blocking Formation of A1 Reactive Astrocytes Preserves Neuronal Health in a Model of Spinal Cord Injury

Weight-drop spinal cord injury (SCI) is a well-established model for studying SCI in mice and making inferences about SCI treatments and outcomes in humans. A mouse model of weight-drop SCI was employed to evaluate the role of A1 astrocytes in post-injury processes within the CNS. As diagramed in the schematic of FIG. 20A, gene expression analysis was performed at sections of the spinal cord rostral and caudal to the injury as well as at the epicenter of the injury site, accordingly astrocyte activation close to lesion site following weight-drop spinal cord injury in postnatal rats was assessed. The top heatmaps show upregulation (red) of reactive astrocyte markers in individual animals in rostral (FIG. 20A), epicenter (FIG. 20B), and caudal (FIG. 20C) to the lesion site. The bottom heatmaps are averages of four animals in each group. Animals either received sham operation (laminectomy, no crush), vehicle injection (laminectomy, crush, PBS injection), IgG control (laminectomy, crush, control IgG antibody injection), or A1-neutralizing antibody injection (laminectomy, crush, anti-II1α/TNFα/C1q injection). Vehicle and IgG control antibody injected animals showed no change in activation state of astrocytes at any region. Anti-A1 neutralizing antibody injection decreased A1 signature in rostral and caudal regions.

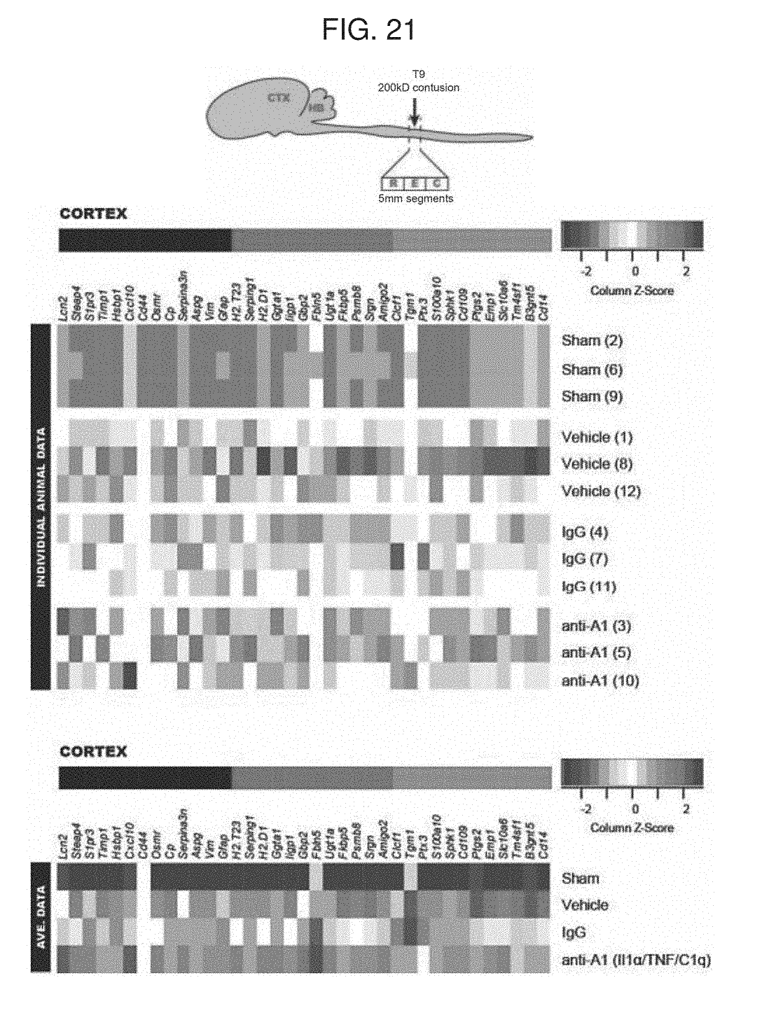

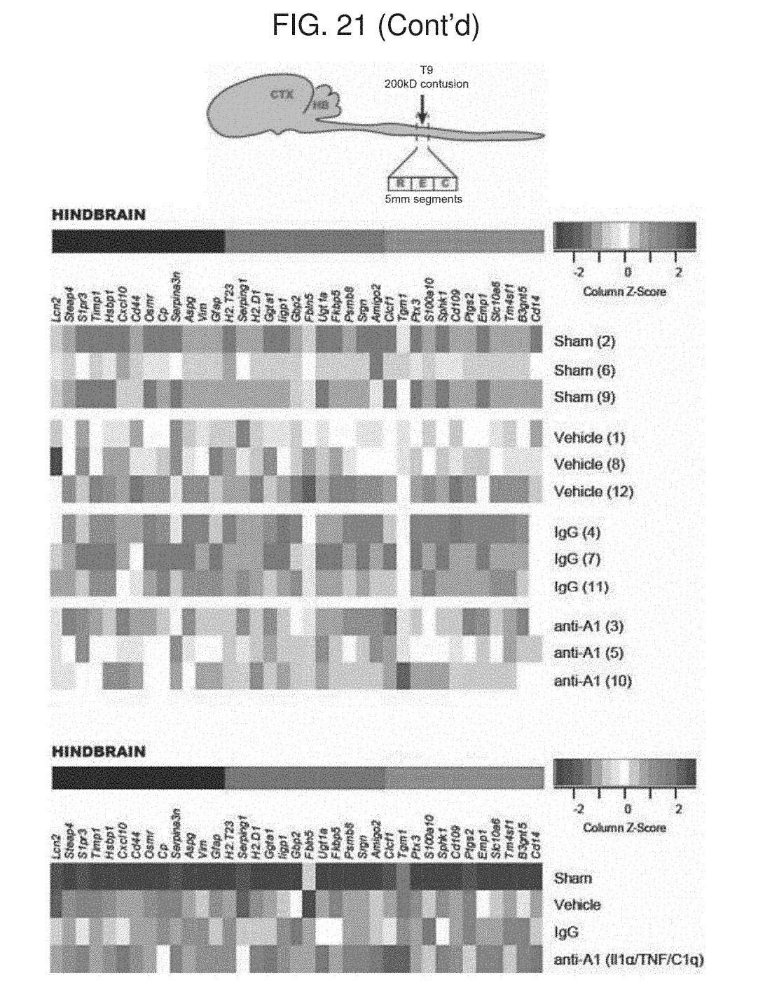

The weight-drop SCI model was further employed to evaluate inhibition of A1 astrocytes in post-injury processes distant from the injury site. Astrocyte activation in the hindbrain and cortex following weight-drop SCI in postnatal rats was evaluated and the data is provided in FIG. 21A-21B, following the same arrangement as FIG. 20A-200 above. As the data shows, acute weight-drop spinal cord injury not only induced A1 astrocytes in the spinal cord locally (see FIG. 20A-200), but also in the hindbrain and cortex. Neutralizing antibody injection into the lesion site (i.e., the spinal cord) did not alter activation of astrocytes in brain regions away from the injury even though such administration was effective at repressing A1 activation in the spinal cord both rostral and caudal to the injury.

Collectively, the data of the instant example show that neuronal health following acute CNS injury, such as SCI, may be preserved by blocking formation of A1 reactive astrocytes. In addition, while local administration of inhibitors may be effective to prevent local A1 astrocyte activation, prevention of A1 astrocyte activation at sites distant from the injury may require specific administration of inhibitors at the distant site. For example, in the case of SCI, administration of inhibitors to regions of the brain may be indicated to prevent eventual A1 astrocyte activation in such regions that is a result of the SCI.

### Example 4: Roles for of A1 Reactive Astrocytes in Stroke

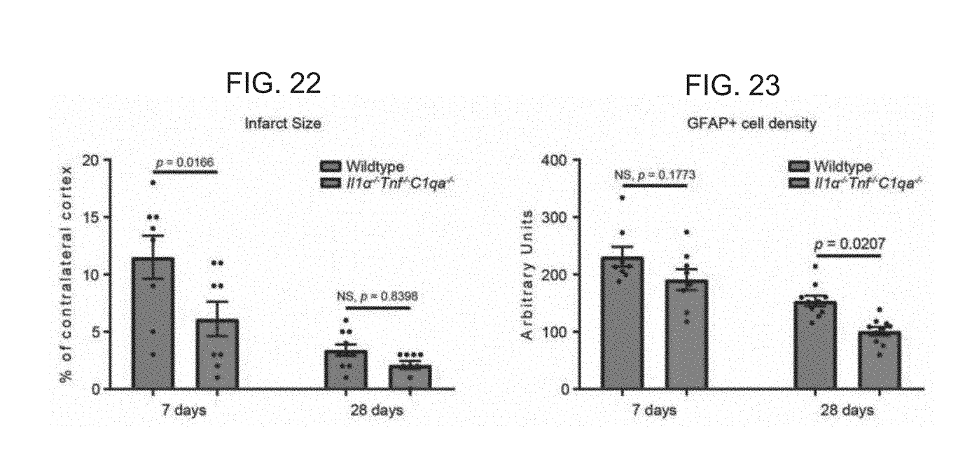

A mouse model of stroke consisting of middle cerebral artery occlusion was used to investigate the influence of A1 reactive astrocytes in short-term and long-term measures in wildtype and II1α−/−Tnf−/−C1qa−/− mice. Infarct size in the two groups was measured at 7 days and 28 days following stroke (FIG. 22). A significant decrease in infarct size was seen in the II1α−/−Tnf−/−C1qa−/− mice as compared to the wildtype mice at the early timepoint (7 days); however, a statistically significant difference in infarct size was not seen at the late timepoint (28 days). Conversely, when GFAP+ cell density was measured, no statistically significant difference was seen between the two groups at the early time point (7 days), but at 28 days following stroke a significant (˜30%) decrease in GFAP signal was seen in the II1α−/−Tnf−/−C1qa−/− mice (FIG. 23). GFAP is used as a broad-spectrum marker for astrocytes, with increases in GFAP immunohistochemistry often used as an indicator of astrocyte reactivity. Accordingly, GFAP+ cells measured may identify A2 “helpful” astrocytes, as well as A1 astrocytes.

Collectively, these data demonstrate that blocking A1 astrocytes formation following stroke results at least in an early decrease in infarct size as compared to animals in which A1 astrocyte formation was not blocked.

Notwithstanding the appended claims, the disclosure is also defined by the following clauses:

1. A method of preventing neuronal or oligodendrocyte death in a subject in need thereof, the method comprising administering to the subject effective amounts of an Interleukin 1 alpha (IL-1α) inhibitor and a tumor necrosis factor alpha (TNFα) inhibitor.

2. The method according to Clause 1, wherein the method further comprises administering to the subject an effective amount of a complement component 1, q subcomponent (C1q) inhibitor.

3. The method according to Clauses 1 or 2, wherein the effective amounts synergistically prevent neuronal death.

4. The method according to any of the preceding Clauses, wherein the subject has a neurodegenerative disease.

5. The method according to Clause 4, wherein the neurodegenerative disease is Alzheimer's disease, Huntington's disease, Parkinson's disease, amyotrophic lateral sclerosis, Multiple Sclerosis, or an eye-related neurodegenerative disease.

6. The method according to any of the preceding Clauses, wherein the subject has a neuroinflammatory disease.

7. The method according to any of the preceding Clauses, wherein the subject has a central nervous system (CNS) injury.

8. The method according to any of the preceding Clauses, wherein the IL-1α inhibitor directly binds IL-1α.

9. The method according to Clause 8, wherein the IL-1α inhibitor is an antibody.

10. The method according to Clause 8, wherein the IL-1α inhibitor is a non-antibody IL-1α antagonist.

11. The method according to any of Clauses 1-7, wherein the IL-1α inhibitor is an antagonist of an IL-1α binding protein that prevents binding of IL-1α to the IL-1α binding protein.

12. The method according to any of the preceding Clauses, wherein the TNFα inhibitor directly binds TNFα.

13. The method according to Clause 12, wherein the TNFα inhibitor is an antibody.

14. The method according to Clause 12, wherein the TNFα inhibitor is a non-antibody TNFα antagonist.

15. The method according to any of Clauses 1-11, wherein the TNFα inhibitor is an antagonist of a TNFα binding protein that prevents binding of TNFα to TNFα binding protein.

16. The method according to any of Clauses 2-15, wherein the C1q inhibitor directly binds C1q.

17. The method according to Clause 16, wherein the C1q inhibitor is an antibody.

18. The method according to Clause 16, wherein the C1q inhibitor is a non-antibody C1q antagonist.

19. The method according to any of Clauses 1-15, wherein the C1q inhibitor is an antagonist of a C1q binding protein that prevents binding of C1q to the C1q binding protein.

20. The method according to any of the preceding Clauses, wherein the subject comprises a population of A1 reactive astrocytes at a site of neurotoxicity.

21. The method according to Clause 20, wherein the A1 reactive astrocytes of the population express one or more A1 reactive astrocyte markers selected from the group consisting of: H2.T23, Serping1, H2.D1, Ggta1, ligp1, Gbp2, FbIn5, Ugt1a, Fkbp5, Psmb8, Srgn, Amigo2 and C3.

22. The method according to Clause 20 or 21, wherein the A1 reactive astrocytes of the population express one or more PAN reactive markers selected from the group consisting of: Lcn2, Steap4, S1pr3, Timp1, Hspb1, Cxcl10, Cd44, Osmr, Cp, Serpina3n, Aspg, Vim and Gfap.

23. The method according to any of Clauses 20-22, wherein the method further comprises identifying the presence of the population of A1 reactive astrocytes.

24. The method according to Clause 23, wherein the identifying comprises detecting the presence of an A1 astrocyte derived neurotoxin in the subject.

25. A neuroprotective composition comprising an effective amount of an IL-1α inhibitor and a TNFα inhibitor.

26. The neuroprotective composition of Clause 25, wherein the composition further comprises a C1q inhibitor.

27. The neuroprotective composition of Clauses 25 or 26, wherein the neuroprotective composition comprises effective amounts that synergistically prevent neuronal death, oligodendrocyte death or a combination thereof.

28. The neuroprotective composition of any of Clauses 25-27, wherein the neuroprotective composition is in unit dosage form.

29. A method of identifying an inhibitor of a neurotoxin, the method comprising: culturing a neuron or oligodendrocyte in a medium conditioned with an A1 reactive astrocyte that produces the neurotoxin; contacting the cultured neuron or oligodendrocyte with a candidate inhibitor; assaying the neuron or oligodendrocyte for viability, wherein when the neuron or oligodendrocyte has increased viability as compared to a control neuron or oligodendrocyte the candidate inhibitor is identified as an inhibitor of the neurotoxin.

30. The method according to Clause 29, wherein the method further includes generating the A1 reactive astrocyte by contacting an astrocyte or a progenitor thereof with IL-1α, TNFα and C1q.

31. The method according to Clauses 29 or 30, wherein the control neuron or oligodendrocyte is cultured in the medium but not contacted with the candidate inhibitor.

32. The method according to any of Clauses 29-31, wherein the A1 reactive astrocyte expresses one or more A1 reactive astrocyte markers selected from the group consisting of: H2.T23, Serping1, H2.D1, Ggta1, ligp1, Gbp2, FbIn5, Ugt1a, Fkbp5, Psmb8, Srgn, Amigo2 and C3.

33. The method according to any of Clauses 28-32, wherein the A1 reactive astrocyte express one or more PAN reactive markers selected from the group consisting of: Lcn2, Steap4, Slpr3, Timp1, Hspb1, Cxcl10, Cd44, Osmr, Cp, Serpina3n, Aspg, Vim and Gfap.

34. The method according to any of Clauses 29-33, wherein the neuron is a central nervous system (CNS) neuron.

35. The method according to Clause 34, wherein the CNS neuron is selected from the group consisting of: a cortical neuron, a spinal motor neuron and a retinal ganglion cell.

36. The method according to any of Clauses 29-35, wherein the neuron or oligodendrocyte is a mammalian neuron or oligodendrocyte.

37. The method according to any of Clauses 29-36, wherein the neurotoxin is heat sensitive.

38. The method according to any of Clauses 29-37, wherein the neurotoxin is protease sensitive.

39. The method according to any of Clauses 29-38, wherein the neurotoxin is greater than 30 kD in size.

40. An inhibitor of a neurotoxin identified according to the method of any of Clauses 29-38.

41. A method of identifying a neurotoxin, the method comprising: generating a medium conditioned with an A1 reactive astrocyte that produces the neurotoxin; purifying the neurotoxin from the conditioned medium; and identifying the purified neurotoxin.

42. The method according to Clause 41, wherein the identifying comprises mass spectrometry.

43. The method according to Clause 41 or 42, wherein the purifying comprises fractionating the conditioned medium into media fractions.

44. The method according to Clause 43, wherein the method comprises assaying the media fractions for neuronal or oligodendrocyte cell killing.

45. The method according to any of Clauses 41-44, wherein the method further comprises assaying the purified neurotoxin for neuronal or oligodendrocyte cell killing.

46. A neurotoxic composition comprising the neurotoxin identified according to any of Clauses 41-45.

47. A method of killing a neuron or oligodendrocyte, the method comprising contacting the neuron or oligodendrocyte with the composition according to Clause 46.

48. A method of identifying a neurotoxic condition in a subject, the method comprising: detecting the level of a neurotoxin identified according to any of Clauses 41-46 in a sample obtained from the subject; and identifying the subject as having a neurotoxic condition when the detected level of the neurotoxin is above a reference level.

49. The method according to Clause 48, wherein the sample comprises cerebrospinal fluid.

50. The method according to Clause 48, wherein the sample comprises blood.

51. The method according to any of Clauses 48-50, wherein the reference level is based on the level of the neurotoxin present in a normal sample.

52. The method according to any of Clauses 48-51, wherein the method further comprises treating the subject for the neurotoxic condition when the subject is identified as having a neurotoxic condition.

53. The method according to Clause 52, wherein the treating comprises a method of preventing neuronal or oligodendrocyte death according to any of Clauses 1-24

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of preventing neuronal or oligodendrocyte death in a subject in need thereof, the method comprising administering to the subject effective amounts of an Interleukin 1 alpha (IL-1α) inhibitor and a tumor necrosis factor alpha (TNFα ) inhibitor.

2. The method according to claim 1, wherein the method further comprises administering to the subject an effective amount of a complement component 1, q subcomponent (C1 q) inhibitor.

3. The method according to claim 1 or 2, wherein the effective amounts synergistically prevent neuronal death.

4. The method according to any of the preceding claims, wherein the subject has a neurodegenerative disease, a neuroinflammatory disease or a central nervous system (CNS) injury.

5. The method according to claim 4, wherein the neurodegenerative disease is Alzheimer's disease, Huntington's disease, Parkinson's disease, amyotrophic lateral sclerosis, Multiple Sclerosis, or an eye-related neurodegenerative disease.

6. The method according to any of the preceding claims, wherein the IL-1α inhibitor directly binds IL-1α.

7. The method according to claim 6, wherein the IL-1α inhibitor is an antibody.

8. The method according to any of the preceding claims, wherein the TNFα inhibitor directly binds TNFα.

9. The method according to claim 8, wherein the TNFα inhibitor is an antibody.

10. The method according to any of claims 2-9, wherein the C1q inhibitor directly binds C1q.

11. The method according to claim 10, wherein the C1q inhibitor is an antibody.

12. A neuroprotective composition comprising an effective amount of an IL-1α inhibitor and a INFα inhibitor.

13. The neuroprotective composition of claim 12, wherein the composition further comprises a C1q inhibitor.

14. The neuroprotective composition of claim 12 or 13, wherein the neuroprotective composition comprises effective amounts that synergistically prevent neuronal death, oligodendrocyte death or a combination thereof.

15. A method of identifying an inhibitor of a neurotoxin, the method comprising:
culturing a neuron or oligodendrocyte in a medium conditioned with an A1 reactive astrocyte that produces the neurotoxin;
contacting the cultured neuron or oligodendrocyte with a candidate inhibitor;
assaying the neuron or oligodendrocyte for viability, wherein when the neuron or oligodendrocyte has increased viability as compared to a control neuron or oligodendrocyte the candidate inhibitor is identified as an inhibitor of the neurotoxin.


SEQUENCE LISTING (256 sequences)

SEQ ID NO: 1 - protein, 15 aa, Artificial sequence
  Synthetic amino acid sequence
CEGPFGPRHDLTFCW

SEQ ID NO: 2 - protein, 15 aa, Artificial sequence
  Synthetic amino acid sequence
LPGVFGGIGDPVTCL

SEQ ID NO: 3 - DNA, 20 bp, Artificial sequence
  Synthetic nucleic acid sequence
aggggctgtttttctctcgg

SEQ ID NO: 4 - DNA, 20 bp, Artificial sequence
  Synthetic nucleic acid sequence
catggtagcaggagggttgg

SEQ ID NO: 5 - DNA, 20 bp, Artificial sequence
  Synthetic nucleic acid sequence
aaaaggggcgcaacaagttc

SEQ ID NO: 6 - DNA, 20 bp, Artificial sequence
  Synthetic nucleic acid sequence
gatgccttccgggttctcaa

SEQ ID NO: 7 - DNA, 23 bp, Artificial sequence
  Synthetic nucleic acid sequence
aaggatttgcagggaggaaaagc

SEQ ID NO: 8 - DNA, 20 bp, Artificial sequence
  Synthetic nucleic acid sequence
agtgaaggagctgtgtgacg

SEQ ID NO: 9 - DNA, 20 bp, Artificial sequence
  Synthetic nucleic acid sequence
gttcgccacaacaacatcac

SEQ ID NO: 10 - DNA, 20 bp, Artificial sequence
  Synthetic nucleic acid sequence
aaccccagaagacagcacct

SEQ ID NO: 11 - DNA, 20 bp, Artificial sequence
  Synthetic nucleic acid sequence
caggtgcccaggttcctatc

SEQ ID NO: 12 - DNA, 21 bp, Artificial sequence
  Synthetic nucleic acid sequence
gacacccccgaggtacttatg

SEQ ID NO: 13 - DNA, 20 bp, Artificial sequence
  Synthetic nucleic acid sequence
tgctcctggatgaaaggtcc

SEQ ID NO: 14 - DNA, 20 bp, Artificial sequence
  Synthetic nucleic acid sequence
gtcgctcacaggtacctcaa

SEQ ID NO: 15 - DNA, 23 bp, Artificial sequence
  Synthetic nucleic acid sequence
tcagaatctaccgaccatgaagc

SEQ ID NO: 16 - DNA, 21 bp, Artificial sequence
  Synthetic nucleic acid sequence
tcaggatagccccacaacaac

SEQ ID NO: 17 - DNA, 20 bp, Artificial sequence
  Synthetic nucleic acid sequence
cgcatcttgtacctgaccca

SEQ ID NO: 18 - DNA, 20 bp, Artificial sequence
  Synthetic nucleic acid sequence
gactcgtgggggatgttagc

SEQ ID NO: 19 - DNA, 20 bp, Artificial sequence
  Synthetic nucleic acid sequence
gatgtttccccaaacgcctg

SEQ ID NO: 20 - DNA, 19 bp, Artificial sequence
  Synthetic nucleic acid sequence
ttcctgcagaagtccccgt

SEQ ID NO: 21 - DNA, 20 bp, Artificial sequence
  Synthetic nucleic acid sequence
tgcaagtctatcctgtccgc

SEQ ID NO: 22 - DNA, 20 bp, Artificial sequence
  Synthetic nucleic acid sequence
accattgccaacgtctggat

SEQ ID NO: 23 - DNA, 20 bp, Artificial sequence
  Synthetic nucleic acid sequence
agggggttaagcgaaaccag

SEQ ID NO: 24 - DNA, 20 bp, Artificial sequence
  Synthetic nucleic acid sequence
tgcagtgtcggcagttgtat

SEQ ID NO: 25 - DNA, 20 bp, Artificial sequence
  Synthetic nucleic acid sequence
tccatgatggctcaaaccgt

SEQ ID NO: 26 - DNA, 20 bp, Artificial sequence
  Synthetic nucleic acid sequence
gtgccagcctcgtctcatag

SEQ ID NO: 27 - DNA, 20 bp, Artificial sequence
  Synthetic nucleic acid sequence
acctcgtccagaagattaac

SEQ ID NO: 28 - DNA, 20 bp, Artificial sequence
  Synthetic nucleic acid sequence
taaaggtccgaggcccaaac

SEQ ID NO: 29 - DNA, 20 bp, Artificial sequence
  Synthetic nucleic acid sequence
aaccgcatcaccattcctgt

SEQ ID NO: 30 - DNA, 21 bp, Artificial sequence
  Synthetic nucleic acid sequence
tctcaggatctgggagttgga

SEQ ID NO: 31 - DNA, 20 bp, Artificial sequence
  Synthetic nucleic acid sequence
ggaagggctcatcagaaggt

SEQ ID NO: 32 - DNA, 21 bp, Artificial sequence
  Synthetic nucleic acid sequence
tggaccgactggttactgatg

SEQ ID NO: 33 - DNA, 20 bp, Artificial sequence
  Synthetic nucleic acid sequence
tttcgaccctacaacccgga

SEQ ID NO: 34 - DNA, 21 bp, Artificial sequence
  Synthetic nucleic acid sequence
atggaaccttccagaagtggg

SEQ ID NO: 35 - DNA, 21 bp, Artificial sequence
  Synthetic nucleic acid sequence
attggagctgttgtgaggagg

SEQ ID NO: 36 - DNA, 20 bp, Artificial sequence
  Synthetic nucleic acid sequence
gagatcactggcaagcacga

SEQ ID NO: 37 - DNA, 20 bp, Artificial sequence
  Synthetic nucleic acid sequence
atttggctcgaagcctttgc

SEQ ID NO: 38 - DNA, 20 bp, Artificial sequence
  Synthetic nucleic acid sequence
gactccgcatttgccctact

SEQ ID NO: 39 - DNA, 20 bp, Artificial sequence
  Synthetic nucleic acid sequence
ccgacactgactacgaccag

SEQ ID NO: 40 - DNA, 21 bp, Artificial sequence
  Synthetic nucleic acid sequence
aggcgtagaggaactatgggt

SEQ ID NO: 41 - DNA, 20 bp, Artificial sequence
  Synthetic nucleic acid sequence
taccgccatggggagaaaac

SEQ ID NO: 42 - DNA, 20 bp, Artificial sequence
  Synthetic nucleic acid sequence
ctgcttctgcgggtttgttc

SEQ ID NO: 43 - DNA, 20 bp, Artificial sequence
  Synthetic nucleic acid sequence
aactccgtgcagaagtcgag

SEQ ID NO: 44 - DNA, 20 bp, Artificial sequence
  Synthetic nucleic acid sequence
aagcacgaagagcgagatgg

SEQ ID NO: 45 - DNA, 22 bp, Artificial sequence
  Synthetic nucleic acid sequence
gtcattctggacatgaagaggt

SEQ ID NO: 46 - DNA, 20 bp, Artificial sequence
  Synthetic nucleic acid sequence
tatctgcggaatggggaacg

SEQ ID NO: 47 - DNA, 20 bp, Artificial sequence
  Synthetic nucleic acid sequence
ctcagccatgcagcaaatcc

SEQ ID NO: 48 - DNA, 20 bp, Artificial sequence
  Synthetic nucleic acid sequence
catcccgttcaggctttgga

SEQ ID NO: 49 - DNA, 20 bp, Artificial sequence
  Synthetic nucleic acid sequence
cccactggctgaaaaggtca

SEQ ID NO: 50 - DNA, 20 bp, Artificial sequence
  Synthetic nucleic acid sequence
gaaagggagttccctgggtt

SEQ ID NO: 51 - DNA, 20 bp, Artificial sequence
  Synthetic nucleic acid sequence
cttgcagaacgagagcctgt

SEQ ID NO: 52 - DNA, 22 bp, Artificial sequence
  Synthetic nucleic acid sequence
gtctttcaggtggtccacaagg

SEQ ID NO: 53 - DNA, 20 bp, Artificial sequence
  Synthetic nucleic acid sequence
tggctcagaggctaactggc

SEQ ID NO: 54 - DNA, 20 bp, Artificial sequence
  Synthetic nucleic acid sequence
tccatagagaccggagcaca

SEQ ID NO: 55 - DNA, 20 bp, Artificial sequence
  Synthetic nucleic acid sequence
ggatgagcaaggcgaacaac

SEQ ID NO: 56 - DNA, 20 bp, Artificial sequence
  Synthetic nucleic acid sequence
gaccctattatggccacgca

SEQ ID NO: 57 - DNA, 20 bp, Artificial sequence
  Synthetic nucleic acid sequence
aaaacgtcctggtcaccttg

SEQ ID NO: 58 - DNA, 21 bp, Artificial sequence
  Synthetic nucleic acid sequence
cagtcccgacaacgtttctct

SEQ ID NO: 59 - DNA, 20 bp, Artificial sequence
  Synthetic nucleic acid sequence
aaagcgagaccctgttccag

SEQ ID NO: 60 - DNA, 22 bp, Artificial sequence
  Synthetic nucleic acid sequence
gttcaaggttatcctgctcgga

SEQ ID NO: 61 - DNA, 20 bp, Artificial sequence
  Synthetic nucleic acid sequence
caaacgccgagtaccttgct

SEQ ID NO: 62 - DNA, 20 bp, Artificial sequence
  Synthetic nucleic acid sequence
agccatagttgcggtccttt

SEQ ID NO: 63 - DNA, 20 bp, Artificial sequence
  Synthetic nucleic acid sequence
agacccaattttcctggggc

SEQ ID NO: 64 - DNA, 20 bp, Artificial sequence
  Synthetic nucleic acid sequence
tcggggcaggaagactatga

SEQ ID NO: 65 - DNA, 20 bp, Artificial sequence
  Synthetic nucleic acid sequence
cgtgagcgatgagaaggaca

SEQ ID NO: 66 - DNA, 20 bp, Artificial sequence
  Synthetic nucleic acid sequence
cgctagagcagataccacga

SEQ ID NO: 67 - DNA, 25 bp, Artificial sequence
  Synthetic nucleic acid sequence
ctgagggacagtaccttctggattc

SEQ ID NO: 68 - DNA, 20 bp, Artificial sequence
  Synthetic nucleic acid sequence
ggaagctgttagtgatcccc

SEQ ID NO: 69 - DNA, 20 bp, Artificial sequence
  Synthetic nucleic acid sequence
gaggagatgagggagttgcg

SEQ ID NO: 70 - DNA, 23 bp, Artificial sequence
  Synthetic nucleic acid sequence
ctccatgtacttcgtcttgaagg

SEQ ID NO: 71 - DNA, 20 bp, Artificial sequence
  Synthetic nucleic acid sequence
tccatccgttgggttgatgg

SEQ ID NO: 72 - DNA, 20 bp, Artificial sequence
  Synthetic nucleic acid sequence
gtttctgcaagtgggagagc

SEQ ID NO: 73 - DNA, 23 bp, Artificial sequence
  Synthetic nucleic acid sequence
acacttacagctgtccagggttg

SEQ ID NO: 74 - DNA, 20 bp, Artificial sequence
  Synthetic nucleic acid sequence
gtccaccttggttgtccgat

SEQ ID NO: 75 - DNA, 19 bp, Artificial sequence
  Synthetic nucleic acid sequence
tgggggttcactcactggg

SEQ ID NO: 76 - DNA, 20 bp, Artificial sequence
  Synthetic nucleic acid sequence
acatgcttgatccgtgtggt

SEQ ID NO: 77 - DNA, 20 bp, Artificial sequence
  Synthetic nucleic acid sequence
ctgtgaagttgagcgttggc

SEQ ID NO: 78 - DNA, 20 bp, Artificial sequence
  Synthetic nucleic acid sequence
ggacactttcctcgtcctgg

SEQ ID NO: 79 - DNA, 21 bp, Artificial sequence
  Synthetic nucleic acid sequence
gactccgtaccaggcatcttc

SEQ ID NO: 80 - DNA, 20 bp, Artificial sequence
  Synthetic nucleic acid sequence
ttctgcgctgagaatgtcca

SEQ ID NO: 81 - DNA, 21 bp, Artificial sequence
  Synthetic nucleic acid sequence
ccccaggtagttcaggtaggt

SEQ ID NO: 82 - DNA, 22 bp, Artificial sequence
  Synthetic nucleic acid sequence
gtagctctgagacgatgcttga

SEQ ID NO: 83 - DNA, 20 bp, Artificial sequence
  Synthetic nucleic acid sequence
ccgaacgtgaagacaaggga

SEQ ID NO: 84 - DNA, 21 bp, Artificial sequence
  Synthetic nucleic acid sequence
acggagctctttttgaccttc

SEQ ID NO: 85 - DNA, 20 bp, Artificial sequence
  Synthetic nucleic acid sequence
tggaacacgaagaccacgag

SEQ ID NO: 86 - DNA, 24 bp, Artificial sequence
  Synthetic nucleic acid sequence
gtgagtatccttttaatcctggca

SEQ ID NO: 87 - DNA, 20 bp, Artificial sequence
  Synthetic nucleic acid sequence
gggtcgcccaagttagaaca

SEQ ID NO: 88 - DNA, 20 bp, Artificial sequence
  Synthetic nucleic acid sequence
tcttcatcacgggcttgtcc

SEQ ID NO: 89 - DNA, 20 bp, Artificial sequence
  Synthetic nucleic acid sequence
agagaaggcagccctggtaa

SEQ ID NO: 90 - DNA, 20 bp, Artificial sequence
  Synthetic nucleic acid sequence
tccgggtgtagttgaggcta

SEQ ID NO: 91 - DNA, 20 bp, Artificial sequence
  Synthetic nucleic acid sequence
aacatatgtggctgggcgaa

SEQ ID NO: 92 - DNA, 21 bp, Artificial sequence
  Synthetic nucleic acid sequence
tccttaatgacctcgccatcc

SEQ ID NO: 93 - DNA, 20 bp, Artificial sequence
  Synthetic nucleic acid sequence
gagttctatggagctcccgc

SEQ ID NO: 94 - DNA, 20 bp, Artificial sequence
  Synthetic nucleic acid sequence
ccgttagcacaatgcggaag

SEQ ID NO: 95 - DNA, 20 bp, Artificial sequence
  Synthetic nucleic acid sequence
ccctggttggctaatccgtt

SEQ ID NO: 96 - DNA, 21 bp, Artificial sequence
  Synthetic nucleic acid sequence
gtctgtgacactgtgctgcat

SEQ ID NO: 97 - DNA, 22 bp, Artificial sequence
  Synthetic nucleic acid sequence
gaagtaagttggagtcggtgga

SEQ ID NO: 98 - DNA, 21 bp, Artificial sequence
  Synthetic nucleic acid sequence
ccacgaggcaactgtcttttc

SEQ ID NO: 99 - DNA, 20 bp, Artificial sequence
  Synthetic nucleic acid sequence
attgtgtgactgctttgggc

SEQ ID NO: 100 - DNA, 20 bp, Artificial sequence
  Synthetic nucleic acid sequence
acggcatttgccagtcctta

SEQ ID NO: 101 - DNA, 21 bp, Artificial sequence
  Synthetic nucleic acid sequence
tgcccacaatgagtggtacag

SEQ ID NO: 102 - DNA, 20 bp, Artificial sequence
  Synthetic nucleic acid sequence
aatgcattggtcggtgggaa

SEQ ID NO: 103 - DNA, 20 bp, Artificial sequence
  Synthetic nucleic acid sequence
tcaccactgtccaatcaggg

SEQ ID NO: 104 - DNA, 20 bp, Artificial sequence
  Synthetic nucleic acid sequence
ttatcagcgcagtgagggac

SEQ ID NO: 105 - DNA, 20 bp, Artificial sequence
  Synthetic nucleic acid sequence
ggctttgcaaggtaagctcg

SEQ ID NO: 106 - DNA, 23 bp, Artificial sequence
  Synthetic nucleic acid sequence
tcactcaaaaggggtttcttagc

SEQ ID NO: 107 - DNA, 20 bp, Artificial sequence
  Synthetic nucleic acid sequence
acctgcgagctctgagagta

SEQ ID NO: 108 - DNA, 20 bp, Artificial sequence
  Synthetic nucleic acid sequence
aatcacagcgttgggtctga

SEQ ID NO: 109 - DNA, 20 bp, Artificial sequence
  Synthetic nucleic acid sequence
aaagtcccggtcccttcttg

SEQ ID NO: 110 - DNA, 20 bp, Artificial sequence
  Synthetic nucleic acid sequence
gggtgggcttcagcagtaat

SEQ ID NO: 111 - DNA, 20 bp, Artificial sequence
  Synthetic nucleic acid sequence
cacagggaaagaagcgaggt

SEQ ID NO: 112 - DNA, 20 bp, Artificial sequence
  Synthetic nucleic acid sequence
ttggtgtgaggggcttagtc

SEQ ID NO: 113 - DNA, 20 bp, Artificial sequence
  Synthetic nucleic acid sequence
cccacttttccatctcggca

SEQ ID NO: 114 - DNA, 20 bp, Artificial sequence
  Synthetic nucleic acid sequence
cctcaacagtccacgagagg

SEQ ID NO: 115 - DNA, 21 bp, Artificial sequence
  Synthetic nucleic acid sequence
gccaatcacagcatagaagcg

SEQ ID NO: 116 - DNA, 24 bp, Artificial sequence
  Synthetic nucleic acid sequence
gaatctgagaaggctctatcccca

SEQ ID NO: 117 - DNA, 20 bp, Artificial sequence
  Synthetic nucleic acid sequence
atgcctgatatgctgcgaca

SEQ ID NO: 118 - DNA, 20 bp, Artificial sequence
  Synthetic nucleic acid sequence
tcctgattattgccccaggc

SEQ ID NO: 119 - DNA, 20 bp, Artificial sequence
  Synthetic nucleic acid sequence
gcccctctaaggtcgggata

SEQ ID NO: 120 - DNA, 20 bp, Artificial sequence
  Synthetic nucleic acid sequence
tgggacaggtacccatcaat

SEQ ID NO: 121 - DNA, 20 bp, Artificial sequence
  Synthetic nucleic acid sequence
ctgtcgactgttcatgggct

SEQ ID NO: 122 - DNA, 20 bp, Artificial sequence
  Synthetic nucleic acid sequence
cagtctgctggttgcatagc

SEQ ID NO: 123 - DNA, 20 bp, Artificial sequence
  Synthetic nucleic acid sequence
aaacaggatcggtcatcggg

SEQ ID NO: 124 - DNA, 20 bp, Artificial sequence
  Synthetic nucleic acid sequence
cagacaaacacctgccgact

SEQ ID NO: 125 - DNA, 20 bp, Artificial sequence
  Synthetic nucleic acid sequence
tcagtcagtccggtttcagc

SEQ ID NO: 126 - DNA, 20 bp, Artificial sequence
  Synthetic nucleic acid sequence
agcgaggaccttccattgtg

SEQ ID NO: 127 - DNA, 20 bp, Artificial sequence
  Synthetic nucleic acid sequence
actgggcagggttgtaatgg

SEQ ID NO: 128 - DNA, 20 bp, Artificial sequence
  Synthetic nucleic acid sequence
cgatctgtgcttggttgtgc

SEQ ID NO: 129 - DNA, 20 bp, Artificial sequence
  Synthetic nucleic acid sequence
ccaggtccgagttgcagaaa

SEQ ID NO: 130 - DNA, 21 bp, Artificial sequence
  Synthetic nucleic acid sequence
ggctaggcctcaacacagtta

SEQ ID NO: 131 - DNA, 20 bp, Artificial sequence
  Synthetic nucleic acid sequence
tgctatgaccaccacttcgt

SEQ ID NO: 132 - DNA, 20 bp, Artificial sequence
  Synthetic nucleic acid sequence
ctgcaatttttctcgcagcc

SEQ ID NO: 133 - DNA, 22 bp, Artificial sequence
  Synthetic nucleic acid sequence
ggatcaacaagcaattcctcga

SEQ ID NO: 134 - DNA, 20 bp, Artificial sequence
  Synthetic nucleic acid sequence
gcaggtacttctgggttgct

SEQ ID NO: 135 - DNA, 20 bp, Artificial sequence
  Synthetic nucleic acid sequence
gaggcgaccataatgtcgtt

SEQ ID NO: 136 - DNA, 20 bp, Artificial sequence
  Synthetic nucleic acid sequence
ctgggcatcctgtcacaaca

SEQ ID NO: 137 - DNA, 20 bp, Artificial sequence
  Synthetic nucleic acid sequence
ttttagggttacggccggtg

SEQ ID NO: 138 - DNA, 20 bp, Artificial sequence
  Synthetic nucleic acid sequence
gctgctggccatttacactg

SEQ ID NO: 139 - DNA, 20 bp, Artificial sequence
  Synthetic nucleic acid sequence
cgtggggcaatgagaactat

SEQ ID NO: 140 - DNA, 20 bp, Artificial sequence
  Synthetic nucleic acid sequence
cactcacctgctgctactca

SEQ ID NO: 141 - DNA, 20 bp, Artificial sequence
  Synthetic nucleic acid sequence
cacagtcgggagccctaaag

SEQ ID NO: 142 - DNA, 20 bp, Artificial sequence
  Synthetic nucleic acid sequence
ggactgatctcagccctctg

SEQ ID NO: 143 - DNA, 20 bp, Artificial sequence
  Synthetic nucleic acid sequence
accttggccaccactcctaa

SEQ ID NO: 144 - DNA, 20 bp, Artificial sequence
  Synthetic nucleic acid sequence
actggtgtagcctagctggt

SEQ ID NO: 145 - DNA, 20 bp, Artificial sequence
  Synthetic nucleic acid sequence
tgcgctttcctcacctactg

SEQ ID NO: 146 - DNA, 20 bp, Artificial sequence
  Synthetic nucleic acid sequence
cttcaatcctcctcgactgg

SEQ ID NO: 147 - DNA, 21 bp, Artificial sequence
  Synthetic nucleic acid sequence
tgtgatgggaatgggcaatga

SEQ ID NO: 148 - DNA, 19 bp, Artificial sequence
  Synthetic nucleic acid sequence
cagcatcgaccggtacctt

SEQ ID NO: 149 - DNA, 20 bp, Artificial sequence
  Synthetic nucleic acid sequence
cccacgtgttgagatcattg

SEQ ID NO: 150 - DNA, 20 bp, Artificial sequence
  Synthetic nucleic acid sequence
gagacactggccagaaaagc

SEQ ID NO: 151 - DNA, 19 bp, Artificial sequence
  Synthetic nucleic acid sequence
cttcagatgcaagcaacaa

SEQ ID NO: 152 - DNA, 20 bp, Artificial sequence
  Synthetic nucleic acid sequence
tatgcttatggctcggctgg

SEQ ID NO: 153 - DNA, 21 bp, Artificial sequence
  Synthetic nucleic acid sequence
gcttcctagcttgcgttcatt

SEQ ID NO: 154 - DNA, 20 bp, Artificial sequence
  Synthetic nucleic acid sequence
aagagggatgctgcccttac

SEQ ID NO: 155 - DNA, 20 bp, Artificial sequence
  Synthetic nucleic acid sequence
ggggtcactgtctgaccact

SEQ ID NO: 156 - DNA, 20 bp, Artificial sequence
  Synthetic nucleic acid sequence
agaaaggttgaatcgctgga

SEQ ID NO: 157 - DNA, 20 bp, Artificial sequence
  Synthetic nucleic acid sequence
agaaaggttgaatcgctgga

SEQ ID NO: 158 - DNA, 20 bp, Artificial sequence
  Synthetic nucleic acid sequence
gtgaacagcatgaggggttt

SEQ ID NO: 159 - DNA, 21 bp, Artificial sequence
  Synthetic nucleic acid sequence
cttgtgcatctccaggtccca

SEQ ID NO: 160 - DNA, 23 bp, Artificial sequence
  Synthetic nucleic acid sequence
tccgagattgtaaagcgtgaaga

SEQ ID NO: 161 - DNA, 19 bp, Artificial sequence
  Synthetic nucleic acid sequence
ggaccgcgaatgacatagc

SEQ ID NO: 162 - DNA, 20 bp, Artificial sequence
  Synthetic nucleic acid sequence
gacatgagcagtcggattga

SEQ ID NO: 163 - DNA, 20 bp, Artificial sequence
  Synthetic nucleic acid sequence
ggggcaatagctcattggta

SEQ ID NO: 164 - DNA, 21 bp, Artificial sequence
  Synthetic nucleic acid sequence
cgcttgagtcggcaaagaaat

SEQ ID NO: 165 - DNA, 21 bp, Artificial sequence
  Synthetic nucleic acid sequence
tgccaccttttgacagtgatg

SEQ ID NO: 166 - DNA, 22 bp, Artificial sequence
  Synthetic nucleic acid sequence
tggcctatacaagcttggcttt

SEQ ID NO: 167 - DNA, 20 bp, Artificial sequence
  Synthetic nucleic acid sequence
ccagttcgccatggtatttt

SEQ ID NO: 168 - DNA, 20 bp, Artificial sequence
  Synthetic nucleic acid sequence
ttctgtcgcatgctcggtta

SEQ ID NO: 169 - DNA, 22 bp, Artificial sequence
  Synthetic nucleic acid sequence
gagaccctcacagcgatccaag

SEQ ID NO: 170 - DNA, 20 bp, Artificial sequence
  Synthetic nucleic acid sequence
caccgaagactggcaggaca

SEQ ID NO: 171 - DNA, 24 bp, Artificial sequence
  Synthetic nucleic acid sequence
ccagcaatgacaaaagagatgaca

SEQ ID NO: 172 - DNA, 20 bp, Artificial sequence
  Synthetic nucleic acid sequence
caaggacgaggtgtcggaaa

SEQ ID NO: 173 - DNA, 20 bp, Artificial sequence
  Synthetic nucleic acid sequence
gtgaaggacccaaagcatgt

SEQ ID NO: 174 - DNA, 20 bp, Artificial sequence
  Synthetic nucleic acid sequence
cagtcctgaagaggcctacg

SEQ ID NO: 175 - DNA, 20 bp, Artificial sequence
  Synthetic nucleic acid sequence
gctgtacaagcagtggcaaa

SEQ ID NO: 176 - DNA, 20 bp, Artificial sequence
  Synthetic nucleic acid sequence
aacaagctctgttgcccatt

SEQ ID NO: 177 - DNA, 20 bp, Artificial sequence
  Synthetic nucleic acid sequence
cctctggctgtggacaaaat

SEQ ID NO: 178 - DNA, 20 bp, Artificial sequence
  Synthetic nucleic acid sequence
aagcctagcgggagagaaac

SEQ ID NO: 179 - DNA, 20 bp, Artificial sequence
  Synthetic nucleic acid sequence
gggtctagagacatgtggcg

SEQ ID NO: 180 - DNA, 20 bp, Artificial sequence
  Synthetic nucleic acid sequence
cctggaggatgtcctttcaa

SEQ ID NO: 181 - DNA, 20 bp, Artificial sequence
  Synthetic nucleic acid sequence
acagccccctctgaattctt

SEQ ID NO: 182 - DNA, 20 bp, Artificial sequence
  Synthetic nucleic acid sequence
gcttcggtggtatgatgctt

SEQ ID NO: 183 - DNA, 20 bp, Artificial sequence
  Synthetic nucleic acid sequence
agcaaggcgaacaactcgat

SEQ ID NO: 184 - DNA, 20 bp, Artificial sequence
  Synthetic nucleic acid sequence
gatgcatgaggtggtgaatg

SEQ ID NO: 185 - DNA, 20 bp, Artificial sequence
  Synthetic nucleic acid sequence
gcaaggttatcctgctcgga

SEQ ID NO: 186 - DNA, 20 bp, Artificial sequence
  Synthetic nucleic acid sequence
cccgaatcgtgtctttccta

SEQ ID NO: 187 - DNA, 21 bp, Artificial sequence
  Synthetic nucleic acid sequence
cgctccagtttccctctgaat

SEQ ID NO: 188 - DNA, 19 bp, Artificial sequence
  Synthetic nucleic acid sequence
ctgttggtcccgtcccaaa

SEQ ID NO: 189 - DNA, 20 bp, Artificial sequence
  Synthetic nucleic acid sequence
agtgatttccccgccaactc

SEQ ID NO: 190 - DNA, 20 bp, Artificial sequence
  Synthetic nucleic acid sequence
gcccaagcatattgtggagt

SEQ ID NO: 191 - DNA, 20 bp, Artificial sequence
  Synthetic nucleic acid sequence
gtgtctaacaggccccagaa

SEQ ID NO: 192 - DNA, 21 bp, Artificial sequence
  Synthetic nucleic acid sequence
tgtgctcagagctttcaacaa

SEQ ID NO: 193 - DNA, 20 bp, Artificial sequence
  Synthetic nucleic acid sequence
cctatgggtcacttgccact

SEQ ID NO: 194 - DNA, 21 bp, Artificial sequence
  Synthetic nucleic acid sequence
agaccagagatggacaggtga

SEQ ID NO: 195 - DNA, 22 bp, Artificial sequence
  Synthetic nucleic acid sequence
ctgagaaagtcagagtagctga

SEQ ID NO: 196 - DNA, 20 bp, Artificial sequence
  Synthetic nucleic acid sequence
ggaaggcacccaaggtcaaa

SEQ ID NO: 197 - DNA, 20 bp, Artificial sequence
  Synthetic nucleic acid sequence
gcatccaacagtccgattct

SEQ ID NO: 198 - DNA, 23 bp, Artificial sequence
  Synthetic nucleic acid sequence
caggaatgtccacacttagacac

SEQ ID NO: 199 - DNA, 20 bp, Artificial sequence
  Synthetic nucleic acid sequence
cctcgaggctgtccttttga

SEQ ID NO: 200 - DNA, 20 bp, Artificial sequence
  Synthetic nucleic acid sequence
gtgggcctgtgcatactctt

SEQ ID NO: 201 - DNA, 20 bp, Artificial sequence
  Synthetic nucleic acid sequence
cccagctgaactgaagaagg

SEQ ID NO: 202 - DNA, 22 bp, Artificial sequence
  Synthetic nucleic acid sequence
gcttggtgacaaaaactacagc

SEQ ID NO: 203 - DNA, 20 bp, Artificial sequence
  Synthetic nucleic acid sequence
gcagcgatttcgatgtccac

SEQ ID NO: 204 - DNA, 20 bp, Artificial sequence
  Synthetic nucleic acid sequence
gcttcagcccagtgaaagac

SEQ ID NO: 205 - DNA, 20 bp, Artificial sequence
  Synthetic nucleic acid sequence
gcagtaggctgaagggttgt

SEQ ID NO: 206 - DNA, 20 bp, Artificial sequence
  Synthetic nucleic acid sequence
ccttgggctataagcggtcc

SEQ ID NO: 207 - DNA, 21 bp, Artificial sequence
  Synthetic nucleic acid sequence
tttctatgtgaagggggctgg

SEQ ID NO: 208 - DNA, 20 bp, Artificial sequence
  Synthetic nucleic acid sequence
tacgtcggagttcagctgtg

SEQ ID NO: 209 - DNA, 25 bp, Artificial sequence
  Synthetic nucleic acid sequence
agtgtatagaggatgttccaggtca

SEQ ID NO: 210 - DNA, 19 bp, Artificial sequence
  Synthetic nucleic acid sequence
gctgcactgtccggttgtt

SEQ ID NO: 211 - DNA, 20 bp, Artificial sequence
  Synthetic nucleic acid sequence
cactgggtaaaggggagtga

SEQ ID NO: 212 - DNA, 20 bp, Artificial sequence
  Synthetic nucleic acid sequence
taaaaggcaagggaatgcac

SEQ ID NO: 213 - DNA, 20 bp, Artificial sequence
  Synthetic nucleic acid sequence
aggcagtgtcagaggcctta

SEQ ID NO: 214 - DNA, 20 bp, Artificial sequence
  Synthetic nucleic acid sequence
cagccttccaggtggacttt

SEQ ID NO: 215 - DNA, 20 bp, Artificial sequence
  Synthetic nucleic acid sequence
aacttgcactctggccctaa

SEQ ID NO: 216 - DNA, 20 bp, Artificial sequence
  Synthetic nucleic acid sequence
tacggccaaatccgttcaca

SEQ ID NO: 217 - DNA, 20 bp, Artificial sequence
  Synthetic nucleic acid sequence
gggaaacctgggatgagatt

SEQ ID NO: 218 - DNA, 20 bp, Artificial sequence
  Synthetic nucleic acid sequence
cggcgatagtcgttagcttc

SEQ ID NO: 219 - DNA, 20 bp, Artificial sequence
  Synthetic nucleic acid sequence
cggcgatagtcgttagcttc

SEQ ID NO: 220 - DNA, 20 bp, Artificial sequence
  Synthetic nucleic acid sequence
gttttgttgcctctgggtgt

SEQ ID NO: 221 - DNA, 20 bp, Artificial sequence
  Synthetic nucleic acid sequence
tgtcagcacaatgcggaaga

SEQ ID NO: 222 - DNA, 20 bp, Artificial sequence
  Synthetic nucleic acid sequence
acagggcagtgcagggatag

SEQ ID NO: 223 - DNA, 20 bp, Artificial sequence
  Synthetic nucleic acid sequence
gcacctcagggtgacttcat

SEQ ID NO: 224 - DNA, 20 bp, Artificial sequence
  Synthetic nucleic acid sequence
ggatggggtgtaggggtact

SEQ ID NO: 225 - DNA, 20 bp, Artificial sequence
  Synthetic nucleic acid sequence
acctcgaagacatccccttt

SEQ ID NO: 226 - DNA, 20 bp, Artificial sequence
  Synthetic nucleic acid sequence
cttcccgttgcttgacgttg

SEQ ID NO: 227 - DNA, 20 bp, Artificial sequence
  Synthetic nucleic acid sequence
tgatgtgctgctgcgagatt

SEQ ID NO: 228 - DNA, 20 bp, Artificial sequence
  Synthetic nucleic acid sequence
aaaggccgttactgaggtgg

SEQ ID NO: 229 - DNA, 20 bp, Artificial sequence
  Synthetic nucleic acid sequence
cacactcaccacccattcag

SEQ ID NO: 230 - DNA, 20 bp, Artificial sequence
  Synthetic nucleic acid sequence
cagatgttcccagagccacc

SEQ ID NO: 231 - DNA, 21 bp, Artificial sequence
  Synthetic nucleic acid sequence
ggaggtggtgttcgaggtgtc

SEQ ID NO: 232 - DNA, 20 bp, Artificial sequence
  Synthetic nucleic acid sequence
ccacagcaaagaggccaatg

SEQ ID NO: 233 - DNA, 20 bp, Artificial sequence
  Synthetic nucleic acid sequence
ctgaagggaggggccatttt

SEQ ID NO: 234 - DNA, 20 bp, Artificial sequence
  Synthetic nucleic acid sequence
tgattgtgtcctgcatggcg

SEQ ID NO: 235 - DNA, 20 bp, Artificial sequence
  Synthetic nucleic acid sequence
gcctaatacctggtgcgtgt

SEQ ID NO: 236 - DNA, 20 bp, Artificial sequence
  Synthetic nucleic acid sequence
cactttcacccaaccgtctt

SEQ ID NO: 237 - DNA, 20 bp, Artificial sequence
  Synthetic nucleic acid sequence
ccccaaagatagcatctgga

SEQ ID NO: 238 - DNA, 20 bp, Artificial sequence
  Synthetic nucleic acid sequence
tcccaaatggaacattggat

SEQ ID NO: 239 - DNA, 20 bp, Artificial sequence
  Synthetic nucleic acid sequence
ctgctcacaagaagcagtgg

SEQ ID NO: 240 - DNA, 20 bp, Artificial sequence
  Synthetic nucleic acid sequence
tcagggaacaattgggagag

SEQ ID NO: 241 - DNA, 20 bp, Artificial sequence
  Synthetic nucleic acid sequence
tctggcatcgctgatgactt

SEQ ID NO: 242 - DNA, 20 bp, Artificial sequence
  Synthetic nucleic acid sequence
ttatcaggaaaggccgattg

SEQ ID NO: 243 - DNA, 20 bp, Artificial sequence
  Synthetic nucleic acid sequence
ggatgctctccaagttgctc

SEQ ID NO: 244 - DNA, 20 bp, Artificial sequence
  Synthetic nucleic acid sequence
ccacaggcttttctggtgat

SEQ ID NO: 245 - DNA, 20 bp, Artificial sequence
  Synthetic nucleic acid sequence
aggccacagcatttgcctaa

SEQ ID NO: 246 - DNA, 20 bp, Artificial sequence
  Synthetic nucleic acid sequence
tgctcgtacccagcatagtg

SEQ ID NO: 247 - DNA, 20 bp, Artificial sequence
  Synthetic nucleic acid sequence
tgggagggccgatgttattg

SEQ ID NO: 248 - DNA, 20 bp, Artificial sequence
  Synthetic nucleic acid sequence
ggcctgagtaatggttgcat

SEQ ID NO: 249 - DNA, 20 bp, Artificial sequence
  Synthetic nucleic acid sequence
ggatgttggttgttcgagcg

SEQ ID NO: 250 - DNA, 20 bp, Artificial sequence
  Synthetic nucleic acid sequence
ggaccttccattgtgcctgg

SEQ ID NO: 251 - DNA, 20 bp, Artificial sequence
  Synthetic nucleic acid sequence
ggggccatcatggtatctgc

SEQ ID NO: 252 - DNA, 20 bp, Artificial sequence
  Synthetic nucleic acid sequence
agggtaggatgtggcacaag

SEQ ID NO: 253 - DNA, 20 bp, Artificial sequence
  Synthetic nucleic acid sequence
agccacgtggtatcaaggag

SEQ ID NO: 254 - DNA, 20 bp, Artificial sequence
  Synthetic nucleic acid sequence
cttgatggtggtgcatgaga

SEQ ID NO: 255 - DNA, 20 bp, Artificial sequence
  Synthetic nucleic acid sequence
aaaaccatgttgggcatgat

SEQ ID NO: 256 - DNA, 20 bp, Artificial sequence
  Synthetic nucleic acid sequence
ttgcgctcctgaaaaactgc